SEQUENCE LISTING (300 sequences)

SEQ ID NO: 1 - protein, 9 aa, Artificial Sequence
  Description of Artificial Sequence Synthetic peptide
RMMRTFMRM

SEQ ID NO: 2 - protein, 9 aa, Artificial Sequence
  Description of Artificial Sequence Synthetic peptide
YMMRTKMRM

SEQ ID NO: 3 - protein, 9 aa, Artificial Sequence
  Description of Artificial Sequence Synthetic peptide
FMMRTKMRM

SEQ ID NO: 4 - protein, 9 aa, Artificial Sequence
  Description of Artificial Sequence Synthetic peptide
RMMRTIMRM

SEQ ID NO: 5 - protein, 9 aa, Artificial Sequence
  Description of Artificial Sequence Synthetic peptide
RMMRTLMRM

SEQ ID NO: 6 - protein, 9 aa, Artificial Sequence
  Description of Artificial Sequence Synthetic peptide
RMMRTVMRM

SEQ ID NO: 7 - protein, 9 aa, Artificial Sequence
  Description of Artificial Sequence Synthetic peptide
RMMRWKMRM

SEQ ID NO: 8 - protein, 9 aa, Artificial Sequence
  Description of Artificial Sequence Synthetic peptide
RMMRTMMRM

SEQ ID NO: 9 - protein, 9 aa, Artificial Sequence
  Description of Artificial Sequence Synthetic peptide
RMMRTKMRK

SEQ ID NO: 10 - protein, 9 aa, Artificial Sequence
  Description of Artificial Sequence Synthetic peptide
RMMDTKMRM

SEQ ID NO: 11 - protein, 9 aa, Artificial Sequence
  Description of Artificial Sequence Synthetic peptide
RMMETKMRM

SEQ ID NO: 12 - protein, 9 aa, Artificial Sequence
  Description of Artificial Sequence Synthetic peptide
RMMRTKMRV

SEQ ID NO: 13 - protein, 9 aa, Artificial Sequence
  Description of Artificial Sequence Synthetic peptide
RMMRTKMRF

SEQ ID NO: 14 - protein, 9 aa, Artificial Sequence
  Description of Artificial Sequence Synthetic peptide
RYMRTKMRM

SEQ ID NO: 15 - protein, 9 aa, Artificial Sequence
  Description of Artificial Sequence Synthetic peptide
RMMRTWMRM

SEQ ID NO: 16 - protein, 9 aa, Artificial Sequence
  Description of Artificial Sequence Synthetic peptide
RMMRTKMRR

SEQ ID NO: 17 - protein, 9 aa, Artificial Sequence
  Description of Artificial Sequence Synthetic peptide
RMMRTKMRY

SEQ ID NO: 18 - protein, 9 aa, Artificial Sequence
  Description of Artificial Sequence Synthetic peptide
RMMRTKMAM

SEQ ID NO: 19 - protein, 9 aa, Artificial Sequence
  Description of Artificial Sequence Synthetic peptide
RPMRTKMRM

SEQ ID NO: 20 - protein, 9 aa, Artificial Sequence
  Description of Artificial Sequence Synthetic peptide
RMMRTKMRW

SEQ ID NO: 21 - protein, 9 aa, Artificial Sequence
  Description of Artificial Sequence Synthetic peptide
RMMRTKMPM

SEQ ID NO: 22 - protein, 9 aa, Artificial Sequence
  Description of Artificial Sequence Synthetic peptide
RMMRTKMSM

SEQ ID NO: 23 - protein, 9 aa, Artificial Sequence
  Description of Artificial Sequence Synthetic peptide
RMMRTKMRH

SEQ ID NO: 24 - protein, 9 aa, Artificial Sequence
  Description of Artificial Sequence Synthetic peptide
RSMRTKMRM

SEQ ID NO: 25 - protein, 9 aa, Artificial Sequence
  Description of Artificial Sequence Synthetic peptide
RTMRTKMRM

SEQ ID NO: 26 - protein, 9 aa, Artificial Sequence
  Description of Artificial Sequence Synthetic peptide
MMMRTKMRM

SEQ ID NO: 27 - protein, 9 aa, Artificial Sequence
  Description of Artificial Sequence Synthetic peptide
RMMRYKMRM

SEQ ID NO: 28 - protein, 9 aa, Artificial Sequence
  Description of Artificial Sequence Synthetic peptide
WMMRTKMRM

SEQ ID NO: 29 - protein, 9 aa, Artificial Sequence
  Description of Artificial Sequence Synthetic peptide
RMMRHKMRM

SEQ ID NO: 30 - protein, 9 aa, Artificial Sequence
  Description of Artificial Sequence Synthetic peptide
RMMRKKMRM

SEQ ID NO: 31 - protein, 9 aa, Artificial Sequence
  Description of Artificial Sequence Synthetic peptide
RMMRRKMRM

SEQ ID NO: 32 - protein, 9 aa, Artificial Sequence
  Description of Artificial Sequence Synthetic peptide
RAMRTKMRM

SEQ ID NO: 33 - protein, 9 aa, Artificial Sequence
  Description of Artificial Sequence Synthetic peptide
RMMRTYMRM

SEQ ID NO: 34 - protein, 9 aa, Artificial Sequence
  Description of Artificial Sequence Synthetic peptide
RMMRTKMYM

SEQ ID NO: 35 - protein, 9 aa, Artificial Sequence
  Description of Artificial Sequence Synthetic peptide
REMRTKMRM

SEQ ID NO: 36 - protein, 9 aa, Artificial Sequence
  Description of Artificial Sequence Synthetic peptide
RRMRTKMRM

SEQ ID NO: 37 - protein, 9 aa, Artificial Sequence
  Description of Artificial Sequence Synthetic peptide
RQMRTKMRM

SEQ ID NO: 38 - protein, 9 aa, Artificial Sequence
  Description of Artificial Sequence Synthetic peptide
RFMRTKMRM

SEQ ID NO: 39 - protein, 9 aa, Artificial Sequence
  Description of Artificial Sequence Synthetic peptide
RWMRTKMRM

SEQ ID NO: 40 - protein, 9 aa, Artificial Sequence
  Description of Artificial Sequence Synthetic peptide
RMMRFKMRM

SEQ ID NO: 41 - protein, 9 aa, Artificial Sequence
  Description of Artificial Sequence Synthetic peptide
RMMRTTMRM

SEQ ID NO: 42 - protein, 9 aa, Artificial Sequence
  Description of Artificial Sequence Synthetic peptide
RMMRTCMRM

SEQ ID NO: 43 - protein, 9 aa, Artificial Sequence
  Description of Artificial Sequence Synthetic peptide
RMMRTNMRM

SEQ ID NO: 44 - protein, 9 aa, Artificial Sequence
  Description of Artificial Sequence Synthetic peptide
RMMRTSMRM

SEQ ID NO: 45 - protein, 9 aa, Artificial Sequence
  Description of Artificial Sequence Synthetic peptide
RMMRTKWRM

SEQ ID NO: 46 - protein, 9 aa, Artificial Sequence
  Description of Artificial Sequence Synthetic peptide
RMMRTKMFM

SEQ ID NO: 47 - protein, 9 aa, Artificial Sequence
  Description of Artificial Sequence Synthetic peptide
MRMRRMRRL

SEQ ID NO: 48 - protein, 9 aa, Artificial Sequence
  Description of Artificial Sequence Synthetic peptide
MRMRRMRRM

SEQ ID NO: 49 - protein, 9 aa, Artificial Sequence
  Description of Artificial Sequence Synthetic peptide
MRMRRMRRI

SEQ ID NO: 50 - protein, 9 aa, Artificial Sequence
  Description of Artificial Sequence Synthetic peptide
MRMRRMRRV

SEQ ID NO: 51 - protein, 9 aa, Artificial Sequence
  Description of Artificial Sequence Synthetic peptide
MRMRRMRRF

SEQ ID NO: 52 - protein, 9 aa, Artificial Sequence
  Description of Artificial Sequence Synthetic peptide
MRMRRMRRY

SEQ ID NO: 53 - protein, 9 aa, Artificial Sequence
  Description of Artificial Sequence Synthetic peptide
MPMRRMRRT

SEQ ID NO: 54 - protein, 9 aa, Artificial Sequence
  Description of Artificial Sequence Synthetic peptide
MLMRRMRRT

SEQ ID NO: 55 - protein, 9 aa, Artificial Sequence
  Description of Artificial Sequence Synthetic peptide
MMMRRMRRT

SEQ ID NO: 56 - protein, 9 aa, Artificial Sequence
  Description of Artificial Sequence Synthetic peptide
MRMRRMRAT

SEQ ID NO: 57 - protein, 9 aa, Artificial Sequence
  Description of Artificial Sequence Synthetic peptide
MRMRRMRPT

SEQ ID NO: 58 - protein, 9 aa, Artificial Sequence
  Description of Artificial Sequence Synthetic peptide
MRMRRMRST

SEQ ID NO: 59 - protein, 9 aa, Artificial Sequence
  Description of Artificial Sequence Synthetic peptide
RRMRRMRRT

SEQ ID NO: 60 - protein, 9 aa, Artificial Sequence
  Description of Artificial Sequence Synthetic peptide
KMRRKMWPA

SEQ ID NO: 61 - protein, 9 aa, Artificial Sequence
  Description of Artificial Sequence Synthetic peptide
KMFRKMSPA

SEQ ID NO: 62 - protein, 9 aa, Artificial Sequence
  Description of Artificial Sequence Synthetic peptide
KMMRKMSPA

SEQ ID NO: 63 - protein, 9 aa, Artificial Sequence
  Description of Artificial Sequence Synthetic peptide
KMIRKMSPA

SEQ ID NO: 64 - protein, 9 aa, Artificial Sequence
  Description of Artificial Sequence Synthetic peptide
KMWRKMSPA

SEQ ID NO: 65 - protein, 9 aa, Artificial Sequence
  Description of Artificial Sequence Synthetic peptide
KMYRKMSPA

SEQ ID NO: 66 - protein, 9 aa, Artificial Sequence
  Description of Artificial Sequence Synthetic peptide
KMLRKMSPA

SEQ ID NO: 67 - protein, 9 aa, Artificial Sequence
  Description of Artificial Sequence Synthetic peptide
KMRRKMSPY

SEQ ID NO: 68 - protein, 9 aa, Artificial Sequence
  Description of Artificial Sequence Synthetic peptide
KMRRKMSPK

SEQ ID NO: 69 - protein, 9 aa, Artificial Sequence
  Description of Artificial Sequence Synthetic peptide
KPRRKMSPA

SEQ ID NO: 70 - protein, 9 aa, Artificial Sequence
  Description of Artificial Sequence Synthetic peptide
KMRRKMSPL

SEQ ID NO: 71 - protein, 9 aa, Artificial Sequence
  Description of Artificial Sequence Synthetic peptide
KMRRKMSPF

SEQ ID NO: 72 - protein, 9 aa, Artificial Sequence
  Description of Artificial Sequence Synthetic peptide
KMRRKMSPM

SEQ ID NO: 73 - protein, 9 aa, Artificial Sequence
  Description of Artificial Sequence Synthetic peptide
FMRRKMSPA

SEQ ID NO: 74 - protein, 9 aa, Artificial Sequence
  Description of Artificial Sequence Synthetic peptide
KMARKMSPA

SEQ ID NO: 75 - protein, 9 aa, Artificial Sequence
  Description of Artificial Sequence Synthetic peptide
KMVRKMSPA

SEQ ID NO: 76 - protein, 9 aa, Artificial Sequence
  Description of Artificial Sequence Synthetic peptide
KMRRKMSPR

SEQ ID NO: 77 - protein, 9 aa, Artificial Sequence
  Description of Artificial Sequence Synthetic peptide
KMRRKMSPW

SEQ ID NO: 78 - protein, 9 aa, Artificial Sequence
  Description of Artificial Sequence Synthetic peptide
YMRRKMSPA

SEQ ID NO: 79 - protein, 9 aa, Artificial Sequence
  Description of Artificial Sequence Synthetic peptide
KMREKMSPA

SEQ ID NO: 80 - protein, 9 aa, Artificial Sequence
  Description of Artificial Sequence Synthetic peptide
KMRDKMSPA

SEQ ID NO: 81 - protein, 9 aa, Artificial Sequence
  Description of Artificial Sequence Synthetic peptide
MMRRKMSPA

SEQ ID NO: 82 - protein, 9 aa, Artificial Sequence
  Description of Artificial Sequence Synthetic peptide
KMNRKMSPA

SEQ ID NO: 83 - protein, 9 aa, Artificial Sequence
  Description of Artificial Sequence Synthetic peptide
KMSRKMSPA

SEQ ID NO: 84 - protein, 9 aa, Artificial Sequence
  Description of Artificial Sequence Synthetic peptide
KMRRFMSPA

SEQ ID NO: 85 - protein, 9 aa, Artificial Sequence
  Description of Artificial Sequence Synthetic peptide
KMRRKMSPV

SEQ ID NO: 86 - protein, 9 aa, Artificial Sequence
  Description of Artificial Sequence Synthetic peptide
RPSCREACL

SEQ ID NO: 87 - protein, 9 aa, Artificial Sequence
  Description of Artificial Sequence Synthetic peptide
RTSCREACK

SEQ ID NO: 88 - protein, 9 aa, Artificial Sequence
  Description of Artificial Sequence Synthetic peptide
RTSCREACW

SEQ ID NO: 89 - protein, 9 aa, Artificial Sequence
  Description of Artificial Sequence Synthetic peptide
RESCREACL

SEQ ID NO: 90 - protein, 9 aa, Artificial Sequence
  Description of Artificial Sequence Synthetic peptide
RTKCREACL

SEQ ID NO: 91 - protein, 9 aa, Artificial Sequence
  Description of Artificial Sequence Synthetic peptide
RTRCREACL

SEQ ID NO: 92 - protein, 9 aa, Artificial Sequence
  Description of Artificial Sequence Synthetic peptide
RTSCRFACL

SEQ ID NO: 93 - protein, 9 aa, Artificial Sequence
  Description of Artificial Sequence Synthetic peptide
RTSCRHACL

SEQ ID NO: 94 - protein, 9 aa, Artificial Sequence
  Description of Artificial Sequence Synthetic peptide
RTSCRRACL

SEQ ID NO: 95 - protein, 9 aa, Artificial Sequence
  Description of Artificial Sequence Synthetic peptide
RTSCRWACL

SEQ ID NO: 96 - protein, 9 aa, Artificial Sequence
  Description of Artificial Sequence Synthetic peptide
RTSCRYACL

SEQ ID NO: 97 - protein, 9 aa, Artificial Sequence
  Description of Artificial Sequence Synthetic peptide
RTSCREACR

SEQ ID NO: 98 - protein, 9 aa, Artificial Sequence
  Description of Artificial Sequence Synthetic peptide
RQSCREACL

SEQ ID NO: 99 - protein, 9 aa, Artificial Sequence
  Description of Artificial Sequence Synthetic peptide
RLSCREACL

SEQ ID NO: 100 - protein, 9 aa, Artificial Sequence
  Description of Artificial Sequence Synthetic peptide
RMSCREACL

SEQ ID NO: 101 - protein, 9 aa, Artificial Sequence
  Description of Artificial Sequence Synthetic peptide
RTSCREACY

SEQ ID NO: 102 - protein, 9 aa, Artificial Sequence
  Description of Artificial Sequence Synthetic peptide
RYSCREACL

SEQ ID NO: 103 - protein, 9 aa, Artificial Sequence
  Description of Artificial Sequence Synthetic peptide
RRSCREACL

SEQ ID NO: 104 - protein, 9 aa, Artificial Sequence
  Description of Artificial Sequence Synthetic peptide
RPKMRMRRM

SEQ ID NO: 105 - protein, 9 aa, Artificial Sequence
  Description of Artificial Sequence Synthetic peptide
RTKMRMRAM

SEQ ID NO: 106 - protein, 9 aa, Artificial Sequence
  Description of Artificial Sequence Synthetic peptide
RTKMRMRPM

SEQ ID NO: 107 - protein, 9 aa, Artificial Sequence
  Description of Artificial Sequence Synthetic peptide
RTMMRMRRM

SEQ ID NO: 108 - protein, 9 aa, Artificial Sequence
  Description of Artificial Sequence Synthetic peptide
RTFMRMRRM

SEQ ID NO: 109 - protein, 9 aa, Artificial Sequence
  Description of Artificial Sequence Synthetic peptide
RTYMRMRRM

SEQ ID NO: 110 - protein, 9 aa, Artificial Sequence
  Description of Artificial Sequence Synthetic peptide
RTKMRMRRR

SEQ ID NO: 111 - protein, 9 aa, Artificial Sequence
  Description of Artificial Sequence Synthetic peptide
RTKMRMRRK

SEQ ID NO: 112 - protein, 9 aa, Artificial Sequence
  Description of Artificial Sequence Synthetic peptide
RTKMRMRRW

SEQ ID NO: 113 - protein, 9 aa, Artificial Sequence
  Description of Artificial Sequence Synthetic peptide
RTWMRMRRM

SEQ ID NO: 114 - protein, 9 aa, Artificial Sequence
  Description of Artificial Sequence Synthetic peptide
RTKMRMRRY

SEQ ID NO: 115 - protein, 9 aa, Artificial Sequence
  Description of Artificial Sequence Synthetic peptide
RQKMRMRRM

SEQ ID NO: 116 - protein, 9 aa, Artificial Sequence
  Description of Artificial Sequence Synthetic peptide
RRKMRMRRM

SEQ ID NO: 117 - protein, 9 aa, Artificial Sequence
  Description of Artificial Sequence Synthetic peptide
RTAMRMRRM

SEQ ID NO: 118 - protein, 9 aa, Artificial Sequence
  Description of Artificial Sequence Synthetic peptide
RTIMRMRRM

SEQ ID NO: 119 - protein, 9 aa, Artificial Sequence
  Description of Artificial Sequence Synthetic peptide
RTLMRMRRM

SEQ ID NO: 120 - protein, 9 aa, Artificial Sequence
  Description of Artificial Sequence Synthetic peptide
RTVMRMRRM

SEQ ID NO: 121 - protein, 9 aa, Artificial Sequence
  Description of Artificial Sequence Synthetic peptide
DTKMRMRRM

SEQ ID NO: 122 - protein, 9 aa, Artificial Sequence
  Description of Artificial Sequence Synthetic peptide
ETKMRMRRM

SEQ ID NO: 123 - protein, 9 aa, Artificial Sequence
  Description of Artificial Sequence Synthetic peptide
FTKMRMRRM

SEQ ID NO: 124 - protein, 9 aa, Artificial Sequence
  Description of Artificial Sequence Synthetic peptide
HTKMRMRRM

SEQ ID NO: 125 - protein, 9 aa, Artificial Sequence
  Description of Artificial Sequence Synthetic peptide
YTKMRMRRM

SEQ ID NO: 126 - protein, 10 aa, Artificial Sequence
  Description of Artificial Sequence Synthetic peptide
FRMRRTRRKM

SEQ ID NO: 127 - protein, 10 aa, Artificial Sequence
  Description of Artificial Sequence Synthetic peptide
YRMRRTRRKM

SEQ ID NO: 128 - protein, 10 aa, Artificial Sequence
  Description of Artificial Sequence Synthetic peptide
RPMRRTRRKM

SEQ ID NO: 129 - protein, 10 aa, Artificial Sequence
  Description of Artificial Sequence Synthetic peptide
RRPRRTRRKM

SEQ ID NO: 130 - protein, 10 aa, Artificial Sequence
  Description of Artificial Sequence Synthetic peptide
MRMRRTRRKM

SEQ ID NO: 131 - protein, 10 aa, Artificial Sequence
  Description of Artificial Sequence Synthetic peptide
WRMRRTRRKM

SEQ ID NO: 132 - protein, 10 aa, Artificial Sequence
  Description of Artificial Sequence Synthetic peptide
RLMRRTRRKM

SEQ ID NO: 133 - protein, 10 aa, Artificial Sequence
  Description of Artificial Sequence Synthetic peptide
RMMRRTRRKM

SEQ ID NO: 134 - protein, 10 aa, Artificial Sequence
  Description of Artificial Sequence Synthetic peptide
RRMRRTRRKR

SEQ ID NO: 135 - protein, 10 aa, Artificial Sequence
  Description of Artificial Sequence Synthetic peptide
RQMRRTRRKM

SEQ ID NO: 136 - protein, 10 aa, Artificial Sequence
  Description of Artificial Sequence Synthetic peptide
RSMRRTRRKM

SEQ ID NO: 137 - protein, 10 aa, Artificial Sequence
  Description of Artificial Sequence Synthetic peptide
RTMRRTRRKM

SEQ ID NO: 138 - protein, 10 aa, Artificial Sequence
  Description of Artificial Sequence Synthetic peptide
RYMRRTRRKM

SEQ ID NO: 139 - protein, 10 aa, Artificial Sequence
  Description of Artificial Sequence Synthetic peptide
REMRRTRRKM

SEQ ID NO: 140 - protein, 10 aa, Artificial Sequence
  Description of Artificial Sequence Synthetic peptide
RKMRRKMSPK

SEQ ID NO: 141 - protein, 10 aa, Artificial Sequence
  Description of Artificial Sequence Synthetic peptide
RPMRRKMSPA

SEQ ID NO: 142 - protein, 10 aa, Artificial Sequence
  Description of Artificial Sequence Synthetic peptide
RKPRRKMSPA

SEQ ID NO: 143 - protein, 10 aa, Artificial Sequence
  Description of Artificial Sequence Synthetic peptide
RKMRRKMSPY

SEQ ID NO: 144 - protein, 10 aa, Artificial Sequence
  Description of Artificial Sequence Synthetic peptide
RKMRRKMSPF

SEQ ID NO: 145 - protein, 10 aa, Artificial Sequence
  Description of Artificial Sequence Synthetic peptide
RKMRRKMSPR

SEQ ID NO: 146 - protein, 10 aa, Artificial Sequence
  Description of Artificial Sequence Synthetic peptide
RKMRRKMSPM

SEQ ID NO: 147 - protein, 10 aa, Artificial Sequence
  Description of Artificial Sequence Synthetic peptide
RRMRRKMSPA

SEQ ID NO: 148 - protein, 10 aa, Artificial Sequence
  Description of Artificial Sequence Synthetic peptide
RLMRRKMSPA

SEQ ID NO: 149 - protein, 10 aa, Artificial Sequence
  Description of Artificial Sequence Synthetic peptide
RMMRRKMSPA

SEQ ID NO: 150 - protein, 10 aa, Artificial Sequence
  Description of Artificial Sequence Synthetic peptide
RKMFRKMSPA

SEQ ID NO: 151 - protein, 10 aa, Artificial Sequence
  Description of Artificial Sequence Synthetic peptide
RKMIRKMSPA

SEQ ID NO: 152 - protein, 10 aa, Artificial Sequence
  Description of Artificial Sequence Synthetic peptide
RKMMRKMSPA

SEQ ID NO: 153 - protein, 10 aa, Artificial Sequence
  Description of Artificial Sequence Synthetic peptide
RKMWRKMSPA

SEQ ID NO: 154 - protein, 10 aa, Artificial Sequence
  Description of Artificial Sequence Synthetic peptide
RKMYRKMSPA

SEQ ID NO: 155 - protein, 10 aa, Artificial Sequence
  Description of Artificial Sequence Synthetic peptide
RKMRRKMWPA

SEQ ID NO: 156 - protein, 10 aa, Artificial Sequence
  Description of Artificial Sequence Synthetic peptide
REMRRKMSPA

SEQ ID NO: 157 - protein, 10 aa, Artificial Sequence
  Description of Artificial Sequence Synthetic peptide
RKMRRKMSPL

SEQ ID NO: 158 - protein, 10 aa, Artificial Sequence
  Description of Artificial Sequence Synthetic peptide
TKMRMRRMRK

SEQ ID NO: 159 - protein, 10 aa, Artificial Sequence
  Description of Artificial Sequence Synthetic peptide
TKMYMRRMRR

SEQ ID NO: 160 - protein, 10 aa, Artificial Sequence
  Description of Artificial Sequence Synthetic peptide
TTMRMRRMRR

SEQ ID NO: 161 - protein, 10 aa, Artificial Sequence
  Description of Artificial Sequence Synthetic peptide
TVMRMRRMRR

SEQ ID NO: 162 - protein, 10 aa, Artificial Sequence
  Description of Artificial Sequence Synthetic peptide
TIMRMRRMRR

SEQ ID NO: 163 - protein, 10 aa, Artificial Sequence
  Description of Artificial Sequence Synthetic peptide
TKMRMRRMRL

SEQ ID NO: 164 - protein, 10 aa, Artificial Sequence
  Description of Artificial Sequence Synthetic peptide
TKMRMRRMRF

SEQ ID NO: 165 - protein, 10 aa, Artificial Sequence
  Description of Artificial Sequence Synthetic peptide
TKMRMRRMRI

SEQ ID NO: 166 - protein, 10 aa, Artificial Sequence
  Description of Artificial Sequence Synthetic peptide
TKMRMRRMRM

SEQ ID NO: 167 - protein, 10 aa, Artificial Sequence
  Description of Artificial Sequence Synthetic peptide
TKMRMRRMRV

SEQ ID NO: 168 - protein, 10 aa, Artificial Sequence
  Description of Artificial Sequence Synthetic peptide
TAMRMRRMRR

SEQ ID NO: 169 - protein, 10 aa, Artificial Sequence
  Description of Artificial Sequence Synthetic peptide
TSMRMRRMRR

SEQ ID NO: 170 - protein, 10 aa, Artificial Sequence
  Description of Artificial Sequence Synthetic peptide
RKMRMRRMRR

SEQ ID NO: 171 - protein, 10 aa, Artificial Sequence
  Description of Artificial Sequence Synthetic peptide
TRMRMRRMRR

SEQ ID NO: 172 - protein, 10 aa, Artificial Sequence
  Description of Artificial Sequence Synthetic peptide
TMMRMRRMRR

SEQ ID NO: 173 - protein, 12 aa, Artificial Sequence
  Description of Artificial Sequence Synthetic peptide
RPRRMRRTRRKM

SEQ ID NO: 174 - protein, 12 aa, Artificial Sequence
  Description of Artificial Sequence Synthetic peptide
RMRRMRRTIRKM

SEQ ID NO: 175 - protein, 12 aa, Artificial Sequence
  Description of Artificial Sequence Synthetic peptide
RMRRMRRTLRKM

SEQ ID NO: 176 - protein, 12 aa, Artificial Sequence
  Description of Artificial Sequence Synthetic peptide
RMRRMRRTMRKM

SEQ ID NO: 177 - protein, 12 aa, Artificial Sequence
  Description of Artificial Sequence Synthetic peptide
RMRRPRRTRRKM

SEQ ID NO: 178 - protein, 12 aa, Artificial Sequence
  Description of Artificial Sequence Synthetic peptide
RMMRMRRTRRKM

SEQ ID NO: 179 - protein, 12 aa, Artificial Sequence
  Description of Artificial Sequence Synthetic peptide
RMRRMRWTRRKM

SEQ ID NO: 180 - protein, 12 aa, Artificial Sequence
  Description of Artificial Sequence Synthetic peptide
RMRRMRRWRRKM

SEQ ID NO: 181 - protein, 12 aa, Artificial Sequence
  Description of Artificial Sequence Synthetic peptide
RMRRMRRTYRKM

SEQ ID NO: 182 - protein, 12 aa, Artificial Sequence
  Description of Artificial Sequence Synthetic peptide
RMPRMRRTRRKM

SEQ ID NO: 183 - protein, 12 aa, Artificial Sequence
  Description of Artificial Sequence Synthetic peptide
RMRPMRRTRRKM

SEQ ID NO: 184 - protein, 10 aa, Artificial Sequence
  Description of Artificial Sequence Synthetic peptide
SPARPRTSCL

SEQ ID NO: 185 - protein, 10 aa, Artificial Sequence
  Description of Artificial Sequence Synthetic peptide
SPARPRTSCF

SEQ ID NO: 186 - protein, 10 aa, Artificial Sequence
  Description of Artificial Sequence Synthetic peptide
SPARPRTSCI

SEQ ID NO: 187 - protein, 10 aa, Artificial Sequence
  Description of Artificial Sequence Synthetic peptide
SPARPRTSCM

SEQ ID NO: 188 - protein, 10 aa, Artificial Sequence
  Description of Artificial Sequence Synthetic peptide
SPARPRTSCV

SEQ ID NO: 189 - protein, 10 aa, Artificial Sequence
  Description of Artificial Sequence Synthetic peptide
SPARPRTSCA

SEQ ID NO: 190 - protein, 10 aa, Artificial Sequence
  Description of Artificial Sequence Synthetic peptide
SPARPRTSIR

SEQ ID NO: 191 - protein, 10 aa, Artificial Sequence
  Description of Artificial Sequence Synthetic peptide
SPARPRTSLR

SEQ ID NO: 192 - protein, 10 aa, Artificial Sequence
  Description of Artificial Sequence Synthetic peptide
SPARPRTSMR

SEQ ID NO: 193 - protein, 10 aa, Artificial Sequence
  Description of Artificial Sequence Synthetic peptide
SPARPRTSVR

SEQ ID NO: 194 - protein, 10 aa, Artificial Sequence
  Description of Artificial Sequence Synthetic peptide
SFARPRTSCR

SEQ ID NO: 195 - protein, 10 aa, Artificial Sequence
  Description of Artificial Sequence Synthetic peptide
STARPRTSCR

SEQ ID NO: 196 - protein, 10 aa, Artificial Sequence
  Description of Artificial Sequence Synthetic peptide
SVARPRTSCR

SEQ ID NO: 197 - protein, 10 aa, Artificial Sequence
  Description of Artificial Sequence Synthetic peptide
SYARPRTSCR

SEQ ID NO: 198 - protein, 10 aa, Artificial Sequence
  Description of Artificial Sequence Synthetic peptide
SPARPRTSYR

SEQ ID NO: 199 - protein, 10 aa, Artificial Sequence
  Description of Artificial Sequence Synthetic peptide
SPARPRTWCR

SEQ ID NO: 200 - protein, 10 aa, Artificial Sequence
  Description of Artificial Sequence Synthetic peptide
SPARPRTSFR

SEQ ID NO: 201 - protein, 10 aa, Artificial Sequence
  Description of Artificial Sequence Synthetic peptide
SIARPRTSCR

SEQ ID NO: 202 - protein, 10 aa, Artificial Sequence
  Description of Artificial Sequence Synthetic peptide
SSARPRTSCR

SEQ ID NO: 203 - protein, 10 aa, Artificial Sequence
  Description of Artificial Sequence Synthetic peptide
SPAFPRTSCR

SEQ ID NO: 204 - protein, 10 aa, Artificial Sequence
  Description of Artificial Sequence Synthetic peptide
SAARPRTSCR

SEQ ID NO: 205 - protein, 10 aa, Artificial Sequence
  Description of Artificial Sequence Synthetic peptide
SPAYPRTSCR

SEQ ID NO: 206 - protein, 10 aa, Artificial Sequence
  Description of Artificial Sequence Synthetic peptide
SPARPRTSCK

SEQ ID NO: 207 - protein, 10 aa, Artificial Sequence
  Description of Artificial Sequence Synthetic peptide
SMARPRTSCR

SEQ ID NO: 208 - protein, 10 aa, Artificial Sequence
  Description of Artificial Sequence Synthetic peptide
SPFRPRTSCR

SEQ ID NO: 209 - protein, 10 aa, Artificial Sequence
  Description of Artificial Sequence Synthetic peptide
SPMRPRTSCR

SEQ ID NO: 210 - protein, 10 aa, Artificial Sequence
  Description of Artificial Sequence Synthetic peptide
SLARPRTSCR

SEQ ID NO: 211 - protein, 10 aa, Artificial Sequence
  Description of Artificial Sequence Synthetic peptide
SQARPRTSCR

SEQ ID NO: 212 - protein, 10 aa, Artificial Sequence
  Description of Artificial Sequence Synthetic peptide
SWARPRTSCR

SEQ ID NO: 213 - protein, 10 aa, Artificial Sequence
  Description of Artificial Sequence Synthetic peptide
SPAWPRTSCR

SEQ ID NO: 214 - protein, 10 aa, Artificial Sequence
  Description of Artificial Sequence Synthetic peptide
SPARPRTFCR

SEQ ID NO: 215 - protein, 10 aa, Artificial Sequence
  Description of Artificial Sequence Synthetic peptide
SPARPRTSCY

SEQ ID NO: 216 - protein, 10 aa, Artificial Sequence
  Description of Artificial Sequence Synthetic peptide
RTKMRMRMMR

SEQ ID NO: 217 - protein, 10 aa, Artificial Sequence
  Description of Artificial Sequence Synthetic peptide
RTKMRMRRMK

SEQ ID NO: 218 - protein, 10 aa, Artificial Sequence
  Description of Artificial Sequence Synthetic peptide
RTKMRMRFMR

SEQ ID NO: 219 - protein, 10 aa, Artificial Sequence
  Description of Artificial Sequence Synthetic peptide
RTKMRMRLMR

SEQ ID NO: 220 - protein, 10 aa, Artificial Sequence
  Description of Artificial Sequence Synthetic peptide
RTKMRMRWMR

SEQ ID NO: 221 - protein, 10 aa, Artificial Sequence
  Description of Artificial Sequence Synthetic peptide
RTKMRMRYMR

SEQ ID NO: 222 - protein, 10 aa, Artificial Sequence
  Description of Artificial Sequence Synthetic peptide
RTKMRMRIMR

SEQ ID NO: 223 - protein, 10 aa, Artificial Sequence
  Description of Artificial Sequence Synthetic peptide
RTKMRMRVMR

SEQ ID NO: 224 - protein, 10 aa, Artificial Sequence
  Description of Artificial Sequence Synthetic peptide
MTKMRMRRMR

SEQ ID NO: 225 - protein, 10 aa, Artificial Sequence
  Description of Artificial Sequence Synthetic peptide
ETKMRMRRMR

SEQ ID NO: 226 - protein, 10 aa, Artificial Sequence
  Description of Artificial Sequence Synthetic peptide
FTKMRMRRMR

SEQ ID NO: 227 - protein, 10 aa, Artificial Sequence
  Description of Artificial Sequence Synthetic peptide
HTKMRMRRMR

SEQ ID NO: 228 - protein, 10 aa, Artificial Sequence
  Description of Artificial Sequence Synthetic peptide
NTKMRMRRMR

SEQ ID NO: 229 - protein, 10 aa, Artificial Sequence
  Description of Artificial Sequence Synthetic peptide
YTKMRMRRMR

SEQ ID NO: 230 - protein, 10 aa, Artificial Sequence
  Description of Artificial Sequence Synthetic peptide
RTKMRMRRMW

SEQ ID NO: 231 - protein, 10 aa, Artificial Sequence
  Description of Artificial Sequence Synthetic peptide
RTKMRMRRMY

SEQ ID NO: 232 - protein, 10 aa, Artificial Sequence
  Description of Artificial Sequence Synthetic peptide
RTKMRMRRMF

SEQ ID NO: 233 - protein, 10 aa, Artificial Sequence
  Description of Artificial Sequence Synthetic peptide
RTKMRMRRMM

SEQ ID NO: 234 - protein, 10 aa, Artificial Sequence
  Description of Artificial Sequence Synthetic peptide
RTKMRMRRMI

SEQ ID NO: 235 - protein, 10 aa, Artificial Sequence
  Description of Artificial Sequence Synthetic peptide
RTKMRMRRML

SEQ ID NO: 236 - protein, 10 aa, Artificial Sequence
  Description of Artificial Sequence Synthetic peptide
RTKMRMRRMV

SEQ ID NO: 237 - protein, 9 aa, Artificial Sequence
  Description of Artificial Sequence Synthetic peptide
RRMRRTRRL

SEQ ID NO: 238 - protein, 9 aa, Artificial Sequence
  Description of Artificial Sequence Synthetic peptide
RRMRRTRRM

SEQ ID NO: 239 - protein, 9 aa, Artificial Sequence
  Description of Artificial Sequence Synthetic peptide
RRMRRTRRF

SEQ ID NO: 240 - protein, 9 aa, Artificial Sequence
  Description of Artificial Sequence Synthetic peptide
RIMRRTRRK

SEQ ID NO: 241 - protein, 9 aa, Artificial Sequence
  Description of Artificial Sequence Synthetic peptide
RMMRRTRRK

SEQ ID NO: 242 - protein, 9 aa, Artificial Sequence
  Description of Artificial Sequence Synthetic peptide
RTMRRTRRK

SEQ ID NO: 243 - protein, 9 aa, Artificial Sequence
  Description of Artificial Sequence Synthetic peptide
RVMRRTRRK

SEQ ID NO: 244 - protein, 9 aa, Artificial Sequence
  Description of Artificial Sequence Synthetic peptide
RSMRRTRRK

SEQ ID NO: 245 - protein, 9 aa, Artificial Sequence
  Description of Artificial Sequence Synthetic peptide
RLMRRTRRK

SEQ ID NO: 246 - protein, 9 aa, Artificial Sequence
  Description of Artificial Sequence Synthetic peptide
RAMRRTRRK

SEQ ID NO: 247 - protein, 9 aa, Artificial Sequence
  Description of Artificial Sequence Synthetic peptide
RQMRRTRRK

SEQ ID NO: 248 - protein, 9 aa, Artificial Sequence
  Description of Artificial Sequence Synthetic peptide
RFMRRTRRK

SEQ ID NO: 249 - protein, 9 aa, Artificial Sequence
  Description of Artificial Sequence Synthetic peptide
RWMRRTRRK

SEQ ID NO: 250 - protein, 9 aa, Artificial Sequence
  Description of Artificial Sequence Synthetic peptide
RYMRRTRRK

SEQ ID NO: 251 - protein, 9 aa, Artificial Sequence
  Description of Artificial Sequence Synthetic peptide
RCMRRTRRK

SEQ ID NO: 252 - protein, 9 aa, Artificial Sequence
  Description of Artificial Sequence Synthetic peptide
RGMRRTRRK

SEQ ID NO: 253 - protein, 9 aa, Artificial Sequence
  Description of Artificial Sequence Synthetic peptide
RNMRRTRRK

SEQ ID NO: 254 - protein, 9 aa, Artificial Sequence
  Description of Artificial Sequence Synthetic peptide
RRMRRTRFK

SEQ ID NO: 255 - protein, 9 aa, Artificial Sequence
  Description of Artificial Sequence Synthetic peptide
RRMRRTRYK

SEQ ID NO: 256 - protein, 9 aa, Artificial Sequence
  Description of Artificial Sequence Synthetic peptide
RRMRRTRRI

SEQ ID NO: 257 - protein, 9 aa, Artificial Sequence
  Description of Artificial Sequence Synthetic peptide
RRMRRTRRV

SEQ ID NO: 258 - protein, 9 aa, Artificial Sequence
  Description of Artificial Sequence Synthetic peptide
RRMRRTRRY

SEQ ID NO: 259 - protein, 9 aa, Artificial Sequence
  Description of Artificial Sequence Synthetic peptide
RRMRRTRRW

SEQ ID NO: 260 - protein, 9 aa, Artificial Sequence
  Description of Artificial Sequence Synthetic peptide
RRMRRTRRA

SEQ ID NO: 261 - protein, 9 aa, Artificial Sequence
  Description of Artificial Sequence Synthetic peptide
RRMRRTRRC

SEQ ID NO: 262 - protein, 9 aa, Artificial Sequence
  Description of Artificial Sequence Synthetic peptide
RRMRRTRRT

SEQ ID NO: 263 - protein, 9 aa, Artificial Sequence
  Description of Artificial Sequence Synthetic peptide
RMMRTKMRM

SEQ ID NO: 264 - protein, 9 aa, Artificial Sequence
  Description of Artificial Sequence Synthetic peptide
MRMRRMRRT

SEQ ID NO: 265 - protein, 9 aa, Artificial Sequence
  Description of Artificial Sequence Synthetic peptide
KMRRKMSPA

SEQ ID NO: 266 - protein, 9 aa, Artificial Sequence
  Description of Artificial Sequence Synthetic peptide
RTSCREACL

SEQ ID NO: 267 - protein, 9 aa, Artificial Sequence
  Description of Artificial Sequence Synthetic peptide
RTKMRMRRM

SEQ ID NO: 268 - protein, 10 aa, Artificial Sequence
  Description of Artificial Sequence Synthetic peptide
RRMRRTRRKM

SEQ ID NO: 269 - protein, 10 aa, Artificial Sequence
  Description of Artificial Sequence Synthetic peptide
RKMRRKMSPA

SEQ ID NO: 270 - protein, 10 aa, Artificial Sequence
  Description of Artificial Sequence Synthetic peptide
TKMRMRRMRR

SEQ ID NO: 271 - protein, 12 aa, Artificial Sequence
  Description of Artificial Sequence Synthetic peptide
RMRRMRRTRRKM

SEQ ID NO: 272 - protein, 10 aa, Artificial Sequence
  Description of Artificial Sequence Synthetic peptide
SPARPRTSCR

SEQ ID NO: 273 - protein, 10 aa, Artificial Sequence
  Description of Artificial Sequence Synthetic peptide
RTKMRMRRMR

SEQ ID NO: 274 - protein, 9 aa, Artificial Sequence
  Description of Artificial Sequence Synthetic peptide
RRMRRTRRK

SEQ ID NO: 275 - protein, 9 aa, Artificial Sequence
  Description of Artificial Sequence Synthetic peptide
  MOD_RES (1)..(1): R, Y, F, M, or W
  MOD_RES (2)..(2): M, Y, P, S, T, A, E, R, Q, F, or W
  MOD_RES (4)..(4): R, D, or E
  MOD_RES (5)..(5): T, W, Y, H, K, R, or F
  MOD_RES (6)..(6): K, F, I, L, V, M, W, Y, T, C, N, or S
  MOD_RES (7)..(7): M or W
  MOD_RES (8)..(8): R, A, P, S, Y, or F
  MOD_RES (9)..(9): M, K, V, F, R, Y, W, or H
XXMXXXXXX

SEQ ID NO: 276 - protein, 9 aa, Artificial Sequence
  Description of Artificial Sequence Synthetic peptide
  MOD_RES (1)..(1): M or R
  MOD_RES (2)..(2): R, P, L, or M
  MOD_RES (8)..(8): R, A, P, or S
  MOD_RES (9)..(9): T, L, M, I, V, F, or Y
XXMRRMRXX

SEQ ID NO: 277 - protein, 9 aa, Artificial Sequence
  Description of Artificial Sequence Synthetic peptide
  MOD_RES (1)..(1): K, F, Y, or M
  MOD_RES (2)..(2): M or P
  MOD_RES (3)..(3): R, F, M, I, W, Y, L, A, V, N, or S
  MOD_RES (4)..(4): R, E, or D
  MOD_RES (5)..(5): K or F
  MOD_RES (7)..(7): S or W
  MOD_RES (9)..(9): A, Y, K, L, F, M, R, W, or V
XXXXXMXPX

SEQ ID NO: 278 - protein, 9 aa, Artificial Sequence
  Description of Artificial Sequence Synthetic peptide
  MOD_RES (2)..(2): T, P, E, Q, L, M, Y, or R
  MOD_RES (3)..(3): S, K, or R
  MOD_RES (6)..(6): E, F, H, R, W, or Y
  MOD_RES (9)..(9): L, K, W, R, or Y
RXXCRXACX

SEQ ID NO: 279 - protein, 9 aa, Artificial Sequence
  Description of Artificial Sequence Synthetic peptide
  MOD_RES (1)..(1): R, D, E, F, H, or Y
  MOD_RES (2)..(2): T, P, Q, or R
  MOD_RES (3)..(3): K, M, F, Y, W, A, I, L, or V
  MOD_RES (8)..(8): R, A, or P
  MOD_RES (9)..(9): M, R, K, W, or Y
XXXMRMRXX

SEQ ID NO: 280 - protein, 10 aa, Artificial Sequence
  Description of Artificial Sequence Synthetic peptide
  MOD_RES (1)..(1): R, F, Y, M, or W
  MOD_RES (2)..(2): R, P, L, M, Q, S, T, Y, or E
  MOD_RES (3)..(3): M or P
  MOD_RES (10)..(10): M or R
XXXRRTRRKX

SEQ ID NO: 281 - protein, 10 aa, Artificial Sequence
  Description of Artificial Sequence Synthetic peptide
  MOD_RES (2)..(2): K, P, R, L, M, or E
  MOD_RES (3)..(3): M or P
  MOD_RES (4)..(4): R, F, I, M, W, or Y
  MOD_RES (8)..(8): S or W
  MOD_RES (10)..(10): A, K, Y, F, R, M, or L
RXXXRKMXPX

SEQ ID NO: 282 - protein, 10 aa, Artificial Sequence
  Description of Artificial Sequence Synthetic peptide
  MOD_RES (1)..(1): T or R
  MOD_RES (2)..(2): K, T, V, I, A, S, R, or M
  MOD_RES (4)..(4): R or Y
  MOD_RES (10)..(10): R, K, L, F, I, M, or V
XXMXMRRMRX

SEQ ID NO: 283 - protein, 12 aa, Artificial Sequence
  Description of Artificial Sequence Synthetic peptide
  MOD_RES (2)..(2): M or P
  MOD_RES (3)..(3): R, M, or P
  MOD_RES (4)..(4): R or P
  MOD_RES (5)..(5): M or P
  MOD_RES (7)..(7): R or W
  MOD_RES (8)..(8): T or W
  MOD_RES (9)..(9): R, I, L, M, or Y
RXXXXRXXXRKM

SEQ ID NO: 284 - protein, 10 aa, Artificial Sequence
  Description of Artificial Sequence Synthetic peptide
  MOD_RES (2)..(2): P, F, T, V, Y, I, S, A, M, L, Q, or W
  MOD_RES (3)..(3): A, F, or M
  MOD_RES (4)..(4): R, F, Y, or W
  MOD_RES (8)..(8): S, W, or F
  MOD_RES (9)..(9): C, I, L, M, V, Y, or F
  MOD_RES (10)..(10): R, L, F, I, M, V, A, K, or Y
SXXXPRTXXX

SEQ ID NO: 285 - protein, 10 aa, Artificial Sequence
  Description of Artificial Sequence Synthetic peptide
  MOD_RES (1)..(1): R, M, E, F, H, N, or Y
  MOD_RES (8)..(8): R, M, F, L, W, Y, I, or V
  MOD_RES (10)..(10): R, K, W, Y, F, M, I, L, or V
XTKMRMRXMX

SEQ ID NO: 286 - protein, 9 aa, Artificial Sequence
  Description of Artificial Sequence Synthetic peptide
  MOD_RES (2)..(2): R, I, M, T, V, S, L, A, Q, F, W, Y, C, G, or N
  MOD_RES (8)..(8): R, F, or Y
  MOD_RES (9)..(9): K, L, M, F, I, V, Y, W, A, C, or T
RXMRRTRXX

SEQ ID NO: 287 - protein, 8 aa, Gallus gallus
SIINFEKL

SEQ ID NO: 288 - protein, 44 aa, Homo sapiens
RRMMRTKMRMRRMRRTRRKMRRKMSPARPRTSCREACLQGWTEA

SEQ ID NO: 289 - protein, 10 aa, Homo sapiens
ELAGIGILTV

SEQ ID NO: 290 - DNA, 74 bp, Homo sapiens
ggccgtattggccccgccacctgtgagcgggaggatgatgaggacaaagatgaggatgtaaggccaaacaggcc

SEQ ID NO: 291 - protein, 15 aa, Homo sapiens
RTRRMMRTKMRMRRM

SEQ ID NO: 292 - protein, 15 aa, Homo sapiens
MMRTKMRMRRMRRTR

SEQ ID NO: 293 - protein, 14 aa, Homo sapiens
TKMRMRRMRRTRRK

SEQ ID NO: 294 - protein, 15 aa, Homo sapiens
RMRRMRRTRRKMRRK

SEQ ID NO: 295 - protein, 15 aa, Homo sapiens
MRRTRRKMRRKMSPA

SEQ ID NO: 296 - protein, 15 aa, Homo sapiens
RRKMRRKMSPARPRT

SEQ ID NO: 297 - protein, 15 aa, Homo sapiens
RRKMSPARPRTSCRE

SEQ ID NO: 298 - protein, 15 aa, Homo sapiens
SPARPRTSCREACLQ

SEQ ID NO: 299 - protein, 15 aa, Homo sapiens
PRTSCREACLQGWTE

SEQ ID NO: 300 - protein, 14 aa, Homo sapiens
TSCREACLQGWTEA

(19) United States
(12) Patent Application Publication
(10) Pub. No.: US 2023/0139506 A1
(43) Pub. Date: May 4, 2023

(54) HETEROCLITIC CANCER VACCINES

(71) Applicant: MEMORIAL SLOAN KETTERING CANCER CENTER, New York, NY (US)

(72) Inventors: Mathieu André-Jérôme Gigoux, New York, NY (US); Jedd D. Wolchok, New York, NY (US); Taha Merghoub, Jersey City, NJ (US)

(21) Appl. No.: 17/912,461

(22) PCT Filed: Mar. 17, 2021
(86) PCT No.: PCT/US2021/022679

§ 371 Date: Sep. 16, 2022

Related U.S. Application Data
(60) Provisional application No. 62/990,696, filed on Mar. 17, 2020.

(51) Int. Cl. A61K 39/00; A61K 38/08; A61K 9/00; A61K 9/127; A61K 9/51; A61P 35/00; C12N 15/86

(52) U.S. Cl. CPC A61K 39/0011; A61K 38/08; A61K 9/0019; A61K 9/127; A61K 9/1273; A61K 9/5123; A61P 35/00; C12N 15/86; A61K 38/00

(57) ABSTRACT

In some aspects, the present invention provides heteroclitic CALRMUT peptides designed and selected to elicit an immune response to CALRMUT in subjects with JAK2 mutant-negative myeloproliferative neoplasms, nucleic acid molecules encoding such peptides, compositions comprising such peptides or nucleic acid molecules, and various associated compositions and methods.

### CROSS-REFERENCE TO RELATED APPLICATIONS

This application claims the benefit of priority of U.S. Provisional Patent Application No. 62/990,696 filed on Mar. 17, 2020, the content of which is hereby incorporated by reference in its entirety.

### STATEMENT REGARDING FEDERALLY SPONSORED RESEARCH

This invention was made with government support under CA008748 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 16, 2021, is named MSKCC_045_WO1_SL.txt and is 73,360 bytes in size.

### INCORPORATION BY REFERENCE

For the purposes of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Numbers in superscript or parentheses following text herein refer to the numbered references identified in the “Reference List” section of this patent application.

### BACKGROUND

The majority of JAK2 mutant-negative myeloproliferative neoplasms (MPN) have a disease-initiating frameshift mutation in calreticulin (CALR) resulting in a common 44-amino acid novel C-terminal mutant fragment (CALR^{MUT}), representing an attractive potential source of neoantigens for cancer vaccines. However, prior studies examining CALR^{MUT} fragment immunogenicity found that T cells from CALR^{MUT} MPN patients had diminished immunoreactivity to CALR^{MUT}-derived peptides compared to healthy individuals—even though the peptides were predicted to be immunogenic. Accordingly, there is a need in the art for new approaches to the development of CALR^{MUT} cancer vaccines. The present invention addresses this need.

### SUMMARY OF THE INVENTION

Some of the main aspects of the present invention are summarized below. Additional aspects are described in the Detailed Description of the Invention, Examples, Figures (Drawings), Brief Description of the Figures, and Claims sections of this disclosure. The description in each section of this patent disclosure, regardless of any heading or sub-heading titles, is intended to be read in conjunction with all other sections. Furthermore, the various embodiments described in each section of this disclosure can be combined in various different ways, and all such combinations are intended to fall within the scope of the present invention.

The present invention is based, in part, on certain discoveries that are described in more detail in the “Examples” section of this patent application. In brief, we investigated two independent myeloproliferative neoplasm (MPN) patient cohorts and found that six MHC-I alleles predicted to efficiently bind to multiple CALR^{MUT}-derived peptides are less frequently observed in CALR^{MUT} MPN patients. This strongly pointed to a higher risk of developing CALR^{MUT} MPN in patients lacking these MHC-I alleles and, at the same time, suggested to us that individuals with these MHC-I alleles could potentially control primordial CALR^{MUT}-expressing tumors as part of the immunoediting process. In addition, this suggested to us that CALR^{MUT}-positive MPN patients were unlikely to respond to cancer vaccines composed of the CALR^{MUT} fragment. Therefore, we analyzed the CALR^{MUT} fragment for peptides that could be modified into heteroclitic peptides and designed numerous heteroclitic CALR^{MUT} peptides to serve as more potent anti-CALR^{MUT} immunogens. We tested our heteroclitic CALR^{MUT} peptides in vitro using human peripheral blood mononuclear cells (PBMCs) from healthy donors unable to respond to CALR^{MUT} peptides, and found that the same T cells could be induced to release IFNγ when primed using the heteroclitic peptides that we designed. Then, to verify whether these heteroclitic CALR^{MUT} peptides could control the growth of CALR^{MUT} tumors in vivo, we performed tests in a pre-clinical mouse model. We showed that mice that were unable to mount an immune response against the original CALR^{MUT} fragment, had significantly delayed tumor growth when given a heteroclitic CALR^{MUT} peptide vaccine of the same specificity and that this was further enhanced by PD1 blockade.

Based on these studies, the present invention provides numerous heteroclitic CALR^{MUT} peptides that were specifically designed and selected to elicit an immune response to CALR^{MUT}. The amino acid sequences and SEQ ID NOs of these peptides, as well as those of the parental non-heteroclitic CALR^{MUT} peptides from which they were derived, are provided in Table A and Table B in the Detailed Description section of this patent disclosure. The present invention also provides nucleic acid molecules encoding these peptides, and numerous related compositions and methods, as described further herein.

Accordingly, in some embodiments, the present invention provides an isolated heteroclitic CALR^{MUT} peptide derived from SEQ ID NO. 268, wherein the peptide comprises at least one point mutation as compared to SEQ ID NO. 268. In some such embodiments the heteroclitic CALR^{MUT} peptide is 9-12 amino acids in length.

In some embodiments, the present invention provides an isolated heteroclitic CALR^{MUT} peptide comprising an amino acid sequence selected from the group consisting of SEQ ID NOs 1-262.

Similarly, in some embodiments, the present invention provides an isolated heteroclitic CALR^{MUT} peptide comprising an amino acid sequence selected from the group consisting of SEQ ID NOs 1-46.

In some embodiments, the present invention provides an isolated heteroclitic CALR^{MUT} peptide comprising an amino acid sequence selected from the group consisting of SEQ ID NOs 1, 2, 4, 5, 6, 8 and 40.

In some embodiments, the present invention provides an isolated heteroclitic CALR^{MUT} peptide comprising the amino acid sequence of SEQ ID NO. 40.

In some embodiments, the present invention provides an isolated heteroclitic CALR^{MUT} peptide comprising an amino acid sequence selected from the group consisting of SEQ ID NOs 47-59.

In some embodiments, the present invention provides an isolated heteroclitic CALR^{MUT} peptide comprising an amino acid sequence selected from the group consisting of SEQ ID NOs 60-85.

In some embodiments, the present invention provides an isolated heteroclitic CALR^{MUT} peptide comprising an amino acid sequence selected from the group consisting of SEQ ID NOs 86-103.

In some embodiments, the present invention provides an isolated heteroclitic CALR^{MUT} peptide comprising an amino acid sequence selected from the group consisting of SEQ ID NOs 104-125.

In some embodiments, the present invention provides an isolated heteroclitic CALR^{MUT} peptide comprising an amino acid sequence selected from the group consisting of SEQ ID NOs 126-139.

In some embodiments, the present invention provides an isolated heteroclitic CALR^{MUT} peptide comprising an amino acid sequence selected from the group consisting of SEQ ID NOs 140-157.

In some embodiments, the present invention provides an isolated heteroclitic CALR^{MUT} peptide comprising an amino acid sequence selected from the group consisting of SEQ ID NOs 158-172.

In some embodiments, the present invention provides an isolated heteroclitic CALR^{MUT} peptide comprising an amino acid sequence selected from the group consisting of SEQ ID NOs 173-183.

In some embodiments, the present invention provides an isolated heteroclitic CALR^{MUT} peptide comprising an amino acid sequence selected from the group consisting of SEQ ID NOs 184-215.

In some embodiments, the present invention provides an isolated heteroclitic CALR^{MUT} peptide comprising an amino acid sequence selected from the group consisting of SEQ ID NOs 216-236

In some embodiments, the present invention provides an isolated heteroclitic CALR^{MUT} peptide comprising an amino acid sequence selected from the group consisting of SEQ ID NOs 237-262.

In some embodiments, the present invention provides an isolated peptide comprising a heteroclitic derivative of SEQ ID NO. 263 (CALR9p2). In some embodiments, such heteroclitic derivatives of CALR9p2 comprise a single point mutation selected from K6F, R1Y, R1F, K6I, K6L, K6V, K6M and T5F. Examples of such heteroclitic derivatives of CALR9p2 are those having the amino acid sequence of SEQ ID NO. 1 (K6F), SEQ ID NO. 2 (R1Y), SEQ ID NO. 3 (R1F), SEQ ID NO. 4 (K6I), SEQ ID NO. 5 (K6L), SEQ ID NO. 6 (K6V), SEQ ID NO. 8 (K6M), or SEQ ID NO. 40 (T5F). In some embodiments, such heteroclitic derivatives of CALR9p2 comprise two point-mutations selected from K6F, R1Y, R1F, K6I, K6L, K6V, K6M and T5F.

In some embodiments, the present invention provides an isolated peptide comprising a heteroclitic derivative of SEQ ID NO. 264. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 265. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 266. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 267. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 268. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 269. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 270. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 271. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 272. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 273. In some embodiments, such a heteroclitic derivative comprises a single point mutation. In some embodiments, such a heteroclitic derivative comprises two point mutations.

In some embodiments, the present invention provides an isolated peptide comprising a heteroclitic derivative of SEQ ID NO. 274. In some embodiments, such a heteroclitic derivative comprises a single point mutation. In some embodiments, such a heteroclitic derivative comprises two point mutations.

An analysis of the amino acid sequences of all of these heteroclitic CALR^{MUT} peptides, in comparison to the parental non-heteroclitic CALR^{MUT} peptides from which they were derived, enabled us to deduce certain common features and “consensus” amino acid sequences for our heteroclitic CALR^{MUT} peptides.

Accordingly, in some embodiments the present invention provides an isolated heteroclitic CALR^{MUT} peptide comprising the amino acid sequence:

(SEQ ID NO. 275)
X1X2M3X4X5X6X7X8X9

wherein, independently of each other,
X1 is selected from: R, Y, F, M, and W,
X2 is selected from: M, Y, P, S, T, A, E, R, Q, F, and W,
X4 is selected from: R, D, and E,
X5 is selected from: T, W, Y, H, K, R, and F,
X6 is selected from: K, F, I, L, V, M, W, Y, T, C, N, and S,
X7 is selected from: M, and W,
X8 is selected from: R, A, P, S, Y, and F, and
X9 is selected from: M, K, V, F, R, Y, W, and H, and
and wherein, the amino acid sequence comprises at least one point mutation as compared to CALR9p2 (SEQ ID NO. 263).

And in some embodiments the present invention provides an isolated heteroclitic CALR^{MUT} peptide comprising the amino acid sequence:

(SEQ ID NO. 276)
X1X2M3R4R5M6R7X8X9

wherein, independently of each other,
X1 is selected from: M, and R,
X2 is selected from: R, P, L, and M,
X8 is selected from: R, A, P, and S, and
X9 is selected from: T, L, M, I, V, F, and Y, and
wherein, the amino acid sequence comprises at least one point mutation as compared to CALRp8 (SEQ ID NO. 264).

In other embodiments the present invention provides an isolated heteroclitic CALR^{MUT} peptide comprising the amino acid sequence:

(SEQ ID NO. 277)
X1X2X3X4X5M6X7P8X9

wherein, independently of each other,
X1 is selected from: K, F, Y, and M,
X2 is selected from: M, and P,
X3 is selected from: R, F, M, I, W, Y, L, A, V, N, and S,
X4 is selected from: R, E, and D,
X5 is selected from: K, and F,
X7 is selected from: S, and W, and
X9 is selected from: A, Y, K, L, F, M, R, W, and V, and
wherein, the amino acid sequence comprises at least one point mutation as compared to CALR9p19 (SEQ ID NO. 265).

In yet further embodiments the present invention provides an isolated heteroclitic CALR^{MUT} peptide comprising the amino acid sequence:

(SEQ ID NO. 278)
R1X2X3C4R5X6A7C8X9

wherein, independently of each other,
X2 is selected from: T, P, E, Q, L, M, Y, and R,
X3 is selected from: S, K, and R,
X6 is selected from: E, F, H, R, W, and Y, and
X9 is selected from: L, K, W, R, and Y, and
wherein, the amino acid sequence comprises at least one point mutation as compared to CALR9p30 (SEQ ID NO. 266).

In other embodiments the present invention provides an isolated heteroclitic CALR^{MUT} peptide comprising the amino acid sequence:

(SEQ ID NO. 279)
X1X2X3M4R5M6R7X8X9

wherein, independently of each other,
X1 is selected from: R, D, E, F, H, and Y,
X2 is selected from: T, P, Q, and R,
X3 is selected from: K, M, F, Y, W, A, I, L, and V,
X8 is selected from: R, A, and P, and
X9 is selected from: M, R, K, W, and Y, and
wherein, the amino acid sequence comprises at least one point mutation as compared to CALR9p5 (SEQ ID NO. 267).

In some embodiments the present invention provides an isolated heteroclitic CALR^{MUT} peptide comprising the amino acid sequence:

(SEQ ID NO. 280)
X1X2X3R4R5T6R7R8K9X10

wherein, independently of each other,
X1 is selected from: R, F, Y, M, and W,
X2 is selected from: R, P, L, M, Q, S, T, Y, and E,
X3 is selected from: M, and P, and
X10 is selected from: M, and R, and
wherein, the amino acid sequence comprises at least one point mutation as compared to CALR10p11 (SEQ ID NO. 268).

In some embodiments the present invention provides an isolated heteroclitic CALR^{MUT} peptide comprising the amino acid sequence:

(SEQ ID NO. 281)
R1X2X3X4R5K6M7X8P9X10

wherein, independently of each other,
X2 is selected from: K, P, R, L, M, and E,
X3 is selected from: M, and P,
X4 is selected from: R, F, I, M, W, and Y,
X8 is selected from: S, and W, and
X10 is selected from: A, K, Y, F, R, M, and L, and
wherein, the amino acid sequence comprises at least one point mutation as compared to CALR10p18 (SEQ ID NO. 269).

In some embodiments the present invention provides an isolated heteroclitic CALR^{MUT} peptide comprising the amino acid sequence:

(SEQ ID NO. 282)
X1X2M3X4M5R6R7M8R9X10

wherein, independently of each other,
X1 is selected from: T, and R,
X2 is selected from: K, T, V, I, A, S, R, and M,
X4 is selected from: R, and Y, and
X10 is selected from: R, K, L, F, I, M, and V, and
wherein, the amino acid sequence comprises at least one point mutation as compared to CALR10p6 (SEQ ID NO. 270).

In some embodiments the present invention provides an isolated heteroclitic CALR^{MUT} peptide comprising the amino acid sequence:

(SEQ ID NO. 283)
R1X2X3X4X5R6X7X8X9R10K11M12

wherein, independently of each other,
X2 is selected from: M, and P,
X3 is selected from: R, M, and P,
X4 is selected from: R, and P,
X5 is selected from: M, and P,
X7 is selected from: R, and W,
X8 is selected from: T, and W, and
X9 is selected from: R, I, L, M, and Y, and
wherein, the amino acid sequence comprises at least one point mutation as compared to CALR12p9 (SEQ ID NO. 271).

In some embodiments the present invention provides an isolated heteroclitic CALR^{MUT} peptide comprising the amino acid sequence:

(SEQ ID NO. 284)
S1X2X3X4P5R6T7X8X9X10

wherein, independently of each other,
X2 is selected from: P, F, T, V, Y, I, S, A, M, L, Q, and W,
X3 is selected from: A, F, and M,
X4 is selected from: R, F, Y, and W,
X8 is selected from: S, W, and F,
X9 is selected from: C, I, L, M, V, Y, and F, and
X10 is selected from: R, L, F, I, M, V, A, K, and Y, and
wherein, the amino acid sequence comprises at least one point mutation as compared to CALR10p25 (SEQ ID NO. 272).

In some embodiments the present invention provides an isolated heteroclitic CALR^{MUT} peptide comprising the amino acid sequence:

(SEQ ID NO. 285)
X1T2K3M4R5M6R7X8M9X10

wherein, independently of each other,
X1 is selected from: R, M, E, F, H, N, and Y,
X8 is selected from: R, M, F, L, W, Y, I, and V, and
X10 is selected from: R, K, W, Y, F, M, I, L, and V, and
wherein, the amino acid sequence comprises at least one point mutation as compared to CALR10p5 (SEQ ID NO. 273).

In some embodiments the present invention provides an isolated heteroclitic CALR^{MUT} peptide comprising the amino acid sequence:

(SEQ ID NO. 286)
R1X2M3R4R5T6R7X8X9

wherein, independently of each other,
X2 is selected from: R, I, M, T, V, S, L, A, Q, F, W, Y, C, G, and N,
X8 is selected from: R, F, and Y, and
X9 is selected from: K, L, M, F, I, V, Y, W, A, C, and T, and
wherein, the amino acid sequence comprises at least one point mutation as compared to CALR9p11 (SEQ ID NO. 274).

The present invention also provides nucleic acid molecules that encode the heteroclitic CALR^{MUT} peptides described above and elsewhere herein. For example, in some of such embodiments the present invention provides a nucleic acid molecule comprising a nucleic acid sequence that encodes a heteroclitic CALR^{MUT} peptide as described above and/or elsewhere herein. In some of such embodiments the nucleic acid molecule comprises both a nucleic acid sequence encoding a heteroclitic CALR^{MUT} peptide and a nucleic acid sequence encoding a signal peptide, wherein the nucleic acid sequence encoding the heteroclitic CALR^{MUT} peptide is downstream of the nucleic acid sequence encoding the signal peptide. In some embodiments the nucleic acid molecule is a DNA molecule. In some embodiments the nucleic acid molecule comprises a promoter that is operably linked to the nucleic acid sequence encoding the heteroclitic CALR^{MUT} peptide. In some embodiments the nucleic acid molecule is an RNA molecule. In some embodiments the nucleic acid molecule is an mRNA molecule.

The present invention also provides vectors that comprise nucleic acid molecules that encode the heteroclitic CALR^{MUT} peptides described above and elsewhere herein. In some such embodiments the vectors are viral vectors. In some such embodiments the vectors are selected from the group consisting of: adenoviral vectors, adeno-associated virus vectors, retroviral vectors, lentiviral vectors, alphavirus vectors, and vaccinia virus vectors.

The present invention also provides cells that comprise nucleic acid molecules that encode the heteroclitic CALR^{MUT} peptides described above and elsewhere herein.

In addition to the various heteroclitic CALR^{MUT} peptides, nucleic acid molecules and vectors described above and elsewhere herein, the present invention also provides various compositions comprising such peptides, nucleic acid molecules, or vectors. In some embodiments such compositions comprise one or more carriers suitable for administration to mammalian subjects. In some embodiments such compositions comprise a delivery vehicle, such as a nanoparticle, a lipid nanoparticle, a liposome, a lipid, a lipid encapsulation system, a polymer or a polymersome. In some such embodiments such compositions comprise an adjuvant.

The present invention also provides various methods of treatment. For example, in some embodiments the present invention provides methods of treating JAK2 mutant-negative myeloproliferative neoplasms (MPNs) in subjects in need thereof, such methods involving administering to such subjects an effective amount of a heteroclitic CALR^{MUT} peptide, nucleic acid molecule, vector or composition as described herein. In some such embodiments the subject has a JAK2^{V617F} mutant-negative MPN. In some embodiments such methods involve administering one dose of a heteroclitic CALR^{MUT} peptide, nucleic acid molecule, vector or composition to the subject. In some embodiments such methods involve administering two or more doses of a heteroclitic CALR^{MUT} peptide, nucleic acid molecule, vector or composition to the subject. For example, in some embodiments such treatment methods involve administering a priming dose and one or more booster doses of the heteroclitic CALR^{MUT} peptide, nucleic acid molecule, vector or composition to the subject. In some embodiments such methods also comprise administering an effective amount of an immune checkpoint inhibitor to the subject. Suitable immune checkpoint inhibitors include PD-1, PD-L1, PD-L2 and CTLA-4 inhibitors. In some embodiments the immune checkpoint inhibitor is an anti-PD1 antibody. In some embodiments the treatment methods result in one or more of: (a) an immune response to the JAK2 mutant-negative MPN, (b) a CD8+ T cell response to the JAK2 mutant-negative MPN, (c) an anti-CALR^{MUT} immune response, (d) an anti-CALR^{MUT} CD8+ T cell response, and (e) enhanced sensitivity of the to the JAK2 mutant-negative MPN to immune checkpoint blockade.

While some of the main embodiments of the present invention are summarized above, additional aspects and additional details are provided and described in the Brief Description of the Figures, Detailed Description of the Invention, Examples, Claims, and Figures sections of this patent application. Furthermore, it should be understood that variations and combinations of each of the embodiments described herein are contemplated and are intended to fall within the scope of the present invention.

### BRIEF DESCRIPTION OF THE FIGURES

FIG. 1A-F. MHC-I alleles with predicted binding to CALR^{MUT}-derived peptides are less frequent in CALR^{MUT} MPNs. A) Principal component analysis of MHC-I frequencies from CALR^{MUT} MPN patients, JAK2^{V617F} MPN patients, and US Caucasian population B) Comparison of MHC-I allele frequencies from each group compared to each other in NEUS cohort and C) Danish cohort. Frequencies are expressed as percentages. MHC-I alleles that are over-represented in CALR^{MUT} patients are in darker gray (for both the NEUS cohort and Danish cohort), while MHC-I alleles that are under-represented in CALRMUT patients are in mid-gray (for the NEUS cohort and the Danish cohort). For NEUS cohort, only MHC-I alleles differentially expressed between CALR^{MUT} MPN patients compared to both JAK2^{V617F} MPN patients and US Caucasian population were considered. D) Heatmap of predicted binding to each CALR^{MUT}-derived peptides to each MHC-I allele from B (left panel). MHC-I alleles that are under-represented or over-represented in CALR^{MUT} MPN patients in both cohorts are noted with white or black circles, respectively. Actual MHC-I allele frequencies CALR^{MUT} and JAK2^{V617F} MPN patient populations are also noted (right panel). E) Cohort breakdown of CALR^{MUT} MPN MHC-I allele frequencies of individual institution consisting of the NEUS cohort for the six less frequent MHC-I alleles. F) IFNγ ELISpot of PBMCs from healthy donors that had either at least one (black circles) or zero (white circles) under-represented MHC-I allele expanded with a peptide pool derived from the entire CALR^{MUT} fragment and restimulated after 10 days with either the irrelevant peptide (MOG) or the same CALR^{MUT} fragment peptide pool.

FIG. 2A-F. MHC-I bias is selective against CALR^{MUT} fragment. A) Schema depicting calculation of Patient:Peptide Score (PPS). In brief, single peptides derived from longer sequences were applied to netMHCpan v3 to predict class-I HLA binding against all of six any given patient's class-I HLAs. In each patient-peptide combination, the strongest affinity was registered as the PPS. B) The mean PPS of individual peptides derived from indicated protein sequence (9-10mers) from CALR^{MUT} and JAK2^{V617F} MPN patients and pseudo-population created from expected frequencies of US Caucasian population (NEUS only). Protein sequences are the 44-amino acid CALR^{MUT} sequence (CALR^{MUT-44aa}), the wild-type CALR sequence upstream of CALR^{MUT-44aa} (CALR¹⁻³⁶¹) and the irrelevant foreign antigen neuraminidase (NA) from influenza. Also showing peptides broadly subdivided from predicted possible binding (<10⁴ nM) and predicted non-binding (>10⁴ nM) peptides. C) The difference in mean PPS of each group from FIG. 2B. The Student t test was performed to calculate significance. D) Top ten best predicted mean PPS of CALR^{MUT} MPN patients. Peptide sequences are labeled below and shorthand codes (CALR-length-p-start position) are identified above. E) Breakdown of HLA-I allele frequencies in CALR^{MUT} MPN patients from NEUS cohort or F) Danish cohort, versus the predicted binding affinity to the top peptide CALR9p2.

FIG. 3A-E. Human CD8⁺ T cells activated with heteroclitic CALR9p2 peptides cross-react with CALR9p2 peptide. A) Predicted mean PPS in CALR^{MUT} MPN patients of all single amino-acid substitution of CALR9p2. B) Predicted affinity of all single amino-acid substitution of CALR9p2 to HLA-A*02:01. Seven heteroclitic peptides were chosen for further testing and are identified here only by their amino-acid substitution in the CALR9p2 peptide. C) Binding affinity of CALR9p2 and indicated heteroclitic peptide to the most common MHC-I in the CALR^{MUT} MPN patient cohorts. Shadowed area indicates predicted binding affinity range of 5000-500 nM. D) Percent IFNγ⁺CD8⁺ T cells after primary in vitro stimulation of PBMCs with CALR9p2 or CALR9p2 heteroclitic peptides followed by secondary restimulation with either control (MOG) peptide, CALR9p2 or initial CALR9p2 heteroclitic peptide. E) Summary of responding donor PBMCs to CALR9p2 or CALR9p2 heteroclitic peptides.

FIG. 4A-F. CALRMUT sequence is not immunogenic in C57BL/6J mice. A) The predicted binding affinity of CALRMUT-derived peptides (8-10-mers shown) against all available murine MHC-I alleles. The strongest binding peptide CALR9p2 is identified in the figures (see dark gray bar and arrow). B) H-2Kb stabilization assay using TAP-deficient RMA/S cells was performed for CALR9p2 in the presence and absence of serum. The chicken ovalbumin (OVA)-derived peptide SIINFEKL was used as a positive control. C) Timeline of DNA immunization schedule and CD8+ T cell collection for the experiment in D. D) IFNγ ELISpot depicting secondary reactivity of CD8+ T cells isolated from draining lymph nodes of mice DNA immunized with full-length CALRWT, CALRMUT, and OVA. E) Timeline of peptide immunization and CD8+ T cell collection for the experiment in F. F) IFNγ ELISpot depicting secondary reactivity of CD8+ T cells isolated from draining lymph nodes of mice peptide immunized with adjuvant and DMSO, CALR9p2 or SIINFEKL. Data shown represent results from one repeat of experiments performed at least three times.

FIG. 5A-M. Heteroclitic CALR9p2 peptide vaccine elicits cross-reactive CD8⁺ T cell response against CALR9p2 and controls tumor growth. A) Predicted binding affinity to H-2K^{b} of all single amino-acid substitution variants of CALR9p2. Top predicted peptide CALR9p2(T5F) is shown. B) Cartoon of the expected effect of T5F substitution in CALR9p2 peptide conformational binding into H-2K^{b}. Known dominant anchor sites and minor anchor sites are depicted in red and blue, respectively C) MHC-I stabilization assay using TAP-deficient RMA/S cells was performed for CALR9p2 and CALR9p2(T5F) in absence of serum. SIINFEKL was used as a positive control. Representative results from one repeat of an experiment performed at least three times. D) IFNγ ELISpot depicting secondary reactivity of CD8⁺ T cells isolated from draining lymph nodes of mice peptide immunized with adjuvant and DMSO, CALR9p2 or CALR9p2(T5F). Representative results from one repeat of an experiment performed at least three times. The Student's t test was performed to calculate significance. E) Killing assay of peptide-pulsed B16 cells by CD8⁺ T cells isolated from peptide-immunized mice. Representative results from one repeat of an experiment performed at least three times. The Student's t test was performed to calculate significance. F) Timeline of peptide immunization and tumor implantation for prophylactic vaccine G) RMV/s^{pER-CALR9p2} tumor growth over time following prophylactic peptide immunization for individual tumors or H) averaged up until the second mouse reaching the endpoint. I) Survival of mice following the prophylactic vaccine. J) Timeline of peptide immunization and tumor implantation for therapeutic vaccine and in combination with anti-PD1 therapy. K) Tumor growth over time following therapeutic vaccine for individual tumors or L) averaged up until the second mouse reaching the endpoint. M) Survival of mice following the therapeutic vaccine, with or without anti-PD1 therapy. Data from tumor growth experiments represent results from one repeat of experiments performed twice. Significance for tumor growth experiments was calculated by performing a Student's t test on the area under the curve of each tumor. Significance for survival was calculated by performing a log-rank test.

FIG. 6A-C. MHC-II alleles skewing in CALR^{MUT} MPN patients compared to JAK2^{V617F} MPN patients from NEUS cohort. A) Principal component analysis of HLA-II allele frequencies from CALR^{MUT} MPN patients, JAK2^{V617F} MPN patients and US Caucasian population. B) Volcano plot of Barnard's unconditional test or chi square test P value versus difference in MHC-II allele frequencies. Dotted line represents a P value of 0.05. C) Comparison of HLA-I allele frequencies from each group compared to each other in NEUS cohort. HLA-I alleles with greater (depicted as mid-gray dots) or lesser (depicted as dark-gray dots) are shown here. only HLA-I alleles differentially expressed between CALR^{MUT} MPN patients compared to both JAK2^{V617F} MPN patients and US Caucasian population were considered.

FIG. 7A-B. Contingency analysis of MHC-I allele frequencies in NEUS and Danish cohorts. A) Volcano plot of Barnard's unconditional test or chi square test P value versus difference in MHC-I allele frequencies in NEUS cohort and B) Danish cohort. Dotted line represents a P value of 0.05.

FIG. 8. MHC-I alleles with predicted binding to CALR^{MUT}-derived peptides are less frequent in CALR^{MUT} MPNs. Heatmap of predicted binding to each CALR^{MUT}-derived 10mer peptide to each differentially expressed MHC-I allele. Upper boxes represent MHC-I alleles that are more frequent and lower boxes represent MHC-I alleles that are less frequent—in CALR^{MUT} MPN patients.

FIG. 9. MHC-II alleles with predicted binding to CALR^{MUT}-derived peptides are more frequent in CALR^{MUT} MPNs. Heatmap of predicted binding to each CALR^{MUT}-derived 15mer peptide to each differentially expressed MHC-II allele. Upper boxes represent MHC-I alleles that are more frequent and lower boxes represent MHC-I alleles that are less frequent—in CALR^{MUT} MPN patients.

FIG. 10. CALR9p2 heteroclitic peptides increase HLA-A*02:01 stabilization compared to CALR9p2. MHC-I stabilization assay using human TAP-deficient T2 cells was performed for CALR9p2 and CALR9p2 heteroclitic peptides. The MART1-A2 peptide was used as a positive control.

FIG. 11. Control secondary stimulatory conditions of rapid T cell assay of human healthy donor PBMCs. Graphed results of secondary stimulation MOG, CEFT and PMA+Ionomycin (PMA), of human healthy donors that had received the initial stimulation of DMSO, CEFT, CALR9p2 (9p2) and all pool heteroclitic peptides (hetPool). In some cases, not all conditions could be tested.

FIG. 12. CALR9p2 heteroclitic peptides can mount cross-reactive response to CALR9p2 through HLA-A*02:01. PBMCs from two healthy donors were activated in vitro with CALR9p2 heteroclitic peptides and final restimulation was provided by peptide-pulsed HLA-A*02:01-transduced K562 cells. Reactivity was assessed by intracellular staining for IFNγ and TNFα by flow cytometry. PBMC from two other healthy donors showed no reactivity (not shown).

FIG. 13A-C. CALRMUT does not inhibit antigen processing and presentation. A) Representative flow cytometry image of B16F10 cells presentation of SIINFEKL bound to H-2Kb following co-transfection with OVA and different CALR constructs. B) Quantification of the percentage of B16F10 cells presenting SIINFEKL H-2Kb and C) total expression of H-2Kb. Data shown represent results from one repeat of experiments performed at least three times. A Student's t test was used to determine significance.

FIG. 14A-F. Full-length CALRMUT encoding CALR9p2(T5F) elicits activated antigen-specific CD8+ T cells. A) Depiction and Sanger sequencing validation of site-directed mutagenesis introducing CALR9p2(T5F) variant in the pING-CALRMUT DNA vaccine sequence. Only relevant section of DNA construct is depicted. B) IFNγ ELISpot following co-culture of CD8+ T cells from dLNs of pING or pING-CALRMUT-CALR9p2(T5F)-immunized mice with peptide-pulsed T cell-depleted splenocytes. C) Representative image of CALR9p2(T5F)-tetramer staining of live CD8+ T cells from spleen. D) Quantification of CALR9p2(T5F)-tetramer-positive live CD8+ T cells from C). E) Representative image of CD44, Tim3 and Pd1 levels on CALR9p2(T5F)-tetramer-negative and -positive live CD8+ T cells from spleen of pING-CALRMUT-CALR9p2(T5F)-immunized mice. F). Quantification of CD44HITim3HI and CD44HIPd1HI, as well as geometric MFI of Tim3 and Pd1 of CALR9p2(T5F)-tetramer-negative and -positive live CD8+ T cells from pING-CALRMUT-^{CALR9p2(T5F)}-immunized mice from E). Showing one of two representative experiment. Statistical significance was calculated using the Student t test.

FIG. 15A-F. Demonstration that CALR9p2(T5F)-specific CD8⁺ T cells also recognize CALR9p2 following in vitro secondary restimulation. A) Representative image of CALR9p2(T5F)-tetramer staining on live CD8⁺ T cells from dLNs of d7 peptide-immunized mice. Samples shown are from secondary control restimulation of DMSO-pulsed splenocytes. B) Quantification of live CD8⁺ T cells CALR9p2(T5F)-tetramer staining from A). C) Representative image of IFNγ and TNFα intracellular staining following secondary restimulation with peptide-pulsed splenocytes or PMA/Ionomycin of all, CALR9p2(T5F)-tetramer negative, or CALR9p2(T5F)-tetramer positive live CD8⁺ T cells from CALR9p2(T5F) peptide-immunized mice. D) Quantification of live of IFNγ or TNFα positivity in CALR9p2(T5F)-tetramer negative or positive CD8⁺ T cells from C). E-F) Quantification of E) Pd1 and F) Tim3 surface levels by flow cytometry of CALR9p2(T5F)-tetramer negative or positive of live CD8⁺ T cells from CALR9p2(T5F)-immunized mice restimulated with splenocytes-pulsed with indicated peptides or PMA/Ionomycin. To control for possible staining artifacts, also showing results from background stained CALR9p2(T5F)-tetramer negative or positive live CD8⁺ T cells from DMSO-immunized mice. Experiment shown is representative of experiment performed twice. Statistical significance was calculated using the Student t test.

FIG. 16. CALR9p2 cross-reactivity of CD8⁺ T cells primed by CALR9p2(T5F) diminishes over time and is not maintained by subsequent CALR9p2 boosts. IFNγ ELISpot depicting secondary restimulation of CD8⁺ T cells isolated from draining lymph nodes of hock peptide-immunized at different timepoints. Boost #1 and Boost #2 occur at days 7 and 14, respectively.

### DETAILED DESCRIPTION

The sub-headings provided below, and throughout this patent disclosure, are not intended to denote limitations of the various aspects or embodiments of the invention, which are to be understood by reference to the specification as a whole. For example, this Detailed Description is intended to read in conjunction with, and to expand upon, the description provided in the Summary of the Invention section of this application.

### Definitions & Abbreviations

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systéme International de Unites (SI) accepted form.

Numeric ranges provided herein are inclusive of the numbers defining the range. Where a numeric term is preceded by “about,” the term includes the stated number and values ±10% of the stated number.

Numbers in parentheses or superscript following text in this patent disclosure refer to the numbered references provided in the “Reference List” section at the end of this patent disclosure.

Wherever embodiments are described with the language “comprising,” otherwise analogous embodiments described in terms of “consisting of” and/or “consisting essentially of” are included.

As used herein the abbreviation CALR refers to calreticulin.

As used herein the term CALR^{MUT} refers to the 44-amino acid C-terminal fragment of CALR having the amino acid sequence:

(SEQ ID NO. 288)
RRMMRTKMRMRRMRRTRRKMRRKMSPA
RPRTSCREACLQGWTEA

that is generated in response to various mutations (including several known, specific frameshift mutations) in calreticulin, and also refers to any calreticulin mutation (including frameshift mutation) that results in the generation of this 44-amino acid C-terminal fragment.

As used herein the terms “CALR′ peptide” and “native CALR^{MUT} peptide” and “parental CALR^{MUT} peptide” refer a peptide comprising some portion of the 44-amino acid C-terminal mutant fragment of CALR (CALR^{MUT}), i.e., SEQ ID NO. 288. The CALR^{MUT} peptides described herein are typically about 8-13, e.g., 9-12 amino acids long, but can be longer or shorter.

As used herein the abbreviation CTLA-4 refers to cytotoxic T-lymphocyte-associated protein

The terms “heteroclitic,” “heteroclitic peptide” and “heteroclitic CALR^{MUT} peptide” are used herein consistent with the normal meaning of the term “heteroclitic” in the field of the invention, and, as used herein, refer to a mutated version of a peptide that has superior properties as compared to its non-mutant counterpart. The non-mutant counterparts of the hetereoclitic peptides described herein are sometimes referred to herein as “native” peptides or “parental” peptides or “non-heteroclitic peptides” or “CALR^{MUT} peptides” or “native CALR^{MUT} peptides” or “parental CALR^{MUT} peptides.” The heteroclitic peptides provided herein have at least one amino acid point mutation as compared to the native peptides from which they are derived, and were designed and/or selected to have one or more of the following superior properties: (a) superior immunogenicity as compared to their native counterparts, (b) superior HLA binding (e.g. affinity) as compared to their native counterparts, (c) an HLA-I binding affinity of <500 nm, (d) an HLA-I binding affinity of <100 nm, (d) being a superior T cell receptor (TCR) epitope as compared to their native counterparts, (e) superior (e.g., increased) TCR agonist activity as compared to their native counterparts, (f) superior induction of T cell responses as compared to their native counterparts, and (g) induction of superior (e.g. increased) antigen-specific (i.e. CALRMUT-specific) antitumor immunity as compared to their native counterparts. The term “heteroclitic” may also be further understood with reference to: Gold et al., (2003) “A Single Heteroclitic Epitope Determines Cancer Immunity After Xenogeneic DNA Immunization Against a Tumor Differentiation Antigen,” J. Immunol May 15, 2003, 170 (10) 5188-5194; 13. Solinger et al. (1979), “Lymphocyte response to cytochrome c.; Demonstration of a T-cell heteroclitic proliferative response and identification of a topographic antigenic determinant on pigeon cytochrome c whose immune recognition requires two complementing major histocompatibility complex-linked immune response genes,” J. Exp. Med. 150:830; Wang et al. (1999), “The stimulation of low-affinity, nontolerized clones by heteroclitic antigen analogues causes the breaking of tolerance established to an immunodominant T cell epitope.” J. Exp. Med. 190:983; Dyall et al., (1998). “Heteroclitic immunization induces tumor immunity,” J. Exp. Med. 188:1553; Slansky et al., (2000). “Enhanced antigen-specific antitumor immunity with altered peptide ligands that stabilize the MHC-peptide-TCR complex. Immunity,” 13:529; Bakker et al., (1997), “Analogues of CTL epitopes with improved MHC class-I binding capacity elicit anti-melanoma CTL recognizing the wildtype epitope.” Int. J. Cancer 70.:302; Parkhurst et al., (1996), “Improved induction of melanoma-reactive CTL with peptides from the melanoma antigen gp100 modified at HLA-A*0201-binding residues.” J. Immunol. 157:2539; and Valmori et al., (1998), “Enhanced generation of specific tumor-reactive CTL in vitro by selected Melan-A/MART-1 immunodominant peptide analogues,” J. Immunol. 160:1750.

The abbreviation “HLA” refers to human leukocyte antigen.

In each of the embodiments that involve peptides and/or nucleic acid molecules, the peptides and/or nucleic acid molecules can optionally be in “isolated” form. An “isolated” peptide or nucleic acid molecule is not within a living subject or cell and is typically in a form not found in nature. In some embodiments an isolated peptide or nucleic acid molecule may have been purified to a degree that it is not in a form in which it is found in nature. In some embodiments, a peptide or nucleic acid molecule that is isolated is substantially pure. In some embodiments, a protein or nucleic acid molecule that is isolated has a purity of greater than 75%, or greater than 80%, or greater than 90%, or greater than 95%.

The terms “identical” or “percent identity” in the context of a comparison between two peptides refer to amino acid sequences that are the same (identical) or have a specified percentage of amino acid residues that are the same (percent identity), when compared and aligned (introducing gaps, if necessary) for maximum correspondence, not considering any conservative amino acid substitutions as part of the sequence identity. The percent identity of two peptides can be determined using sequence comparison software or algorithms or by visual inspection. Various algorithms and software are known in the art that can be used to obtain alignments of amino acid sequences and determine identity and/or percentage identity.

As used herein the abbreviation “MPN” refers to myeloproliferative neoplasms.

As used herein the abbreviation “PD-1” refers to Programmed Death 1, which is also known as Programmed Death Protein 1 or Programmed Cell Death Protein 1.

As used herein the abbreviation PD-L1 refers to Programmed Cell Death Ligand 1—which is a ligand for PD-1.

As used herein the abbreviation PD-L2 refers to Programmed Cell Death Ligand 2.

Various other terms are defined elsewhere in this patent disclosure, where used. Furthermore, terms that are not specifically defined herein may be more fully understood in the context in which the terms are used and/or by reference to the specification in its entirety. Where no explicit definition is provided all technical and scientific terms used herein have the meanings commonly understood by those of ordinary skill in the art to which this invention pertains.

### Heteroclitic CALR^{MUT} Peptides

In certain embodiments the present invention provides heteroclitic CALR^{MUT} peptides, including those for which amino acid sequences are provided in the below Tables A and B.

Tables A and B also provide the amino acid sequences of the parental CALR^{MUT} peptides from which the various heteroclitic CALR^{MUT} peptides were derived.

TABLE A
Peptide Sequences

| Hetero- | | | | |
|---|---|---|---|---|
| clitic | Hetero- | Parental | Parental | |
| peptide | clitic | peptide | peptide | Parental |
| aa | peptide | aa | SEQ ID | peptide |
| sequence | SEQ ID NO. | sequence | NO. | Name |
| RMMRTFMRM | 1 | RMMRTKMRM | 263 | CALR9p2 |
| YMMRTKMRM | 2 | RMMRTKMRM | 263 | CALR9p2 |
| FMMRTKMRM | 3 | RMMRTKMRM | 263 | CALR9p2 |
| RMMRTIMRM | 4 | RMMRTKMRM | 263 | CALR9p2 |
| RMMRTLMRM | 5 | RMMRTKMRM | 263 | CALR9p2 |
| RMMRTVMRM | 6 | RMMRTKMRM | 263 | CALR9p2 |
| RMMRWKMRM | 7 | RMMRTKMRM | 263 | CALR9p2 |
| RMMRTMMRM | 8 | RMMRTKMRM | 263 | CALR9p2 |
| RMMRTKMRK | 9 | RMMRTKMRM | 263 | CALR9p2 |
| RMMDTKMRM | 10 | RMMRTKMRM | 263 | CALR9p2 |
| RMMETKMRM | 11 | RMMRTKMRM | 263 | CALR9p2 |
| RMMRTKMRV | 12 | RMMRTKMRM | 263 | CALR9p2 |
| RMMRTKMRF | 13 | RMMRTKMRM | 263 | CALR9p2 |
| RYMRTKMRM | 14 | RMMRTKMRM | 263 | CALR9p2 |
| RMMRTWMRM | 15 | RMMRTKMRM | 263 | CALR9p2 |
| RMMRTKMRR | 16 | RMMRTKMRM | 263 | CALR9p2 |
| RMMRTKMRY | 17 | RMMRTKMRM | 263 | CALR9p2 |
| RMMRTKMAM | 18 | RMMRTKMRM | 263 | CALR9p2 |
| RPMRTKMRM | 19 | RMMRTKMRM | 263 | CALR9p2 |
| RMMRTKMRW | 20 | RMMRTKMRM | 263 | CALR9p2 |
| RMMRTKMPM | 21 | RMMRTKMRM | 263 | CALR9p2 |
| RMMRTKMSM | 22 | RMMRTKMRM | 263 | CALR9p2 |
| RMMRTKMRH | 23 | RMMRTKMRM | 263 | CALR9p2 |
| RSMRTKMRM | 24 | RMMRTKMRM | 263 | CALR9p2 |
| RTMRTKMRM | 25 | RMMRTKMRM | 263 | CALR9p2 |
| MMMRTKMRM | 26 | RMMRTKMRM | 263 | CALR9p2 |
| RMMRYKMRM | 27 | RMMRTKMRM | 263 | CALR9p2 |
| WMMRTKMRM | 28 | RMMRTKMRM | 263 | CALR9p2 |
| RMMRHKMRM | 29 | RMMRTKMRM | 263 | CALR9p2 |
| RMMRKKMRM | 30 | RMMRTKMRM | 263 | CALR9p2 |
| RMMRRKMRM | 31 | RMMRTKMRM | 263 | CALR9p2 |
| RAMRTKMRM | 32 | RMMRTKMRM | 263 | CALR9p2 |
| RMMRTYMRM | 33 | RMMRTKMRM | 263 | CALR9p2 |
| RMMRTKMYM | 34 | RMMRTKMRM | 263 | CALR9p2 |
| REMRTKMRM | 35 | RMMRTKMRM | 263 | CALR9p2 |
| RRMRTKMRM | 36 | RMMRTKMRM | 263 | CALR9p2 |
| RQMRTKMRM | 37 | RMMRTKMRM | 263 | CALR9p2 |
| RFMRTKMRM | 38 | RMMRTKMRM | 263 | CALR9p2 |
| RWMRTKMRM | 39 | RMMRTKMRM | 263 | CALR9p2 |
| RMMRFKMRM | 40 | RMMRTKMRM | 263 | CALR9p2 |
| RMMRTTMRM | 41 | RMMRTKMRM | 263 | CALR9p2 |
| RMMRTCMRM | 42 | RMMRTKMRM | 263 | CALR9p2 |
| RMMRTNMRM | 43 | RMMRTKMRM | 263 | CALR9p2 |
| RMMRTSMRM | 44 | RMMRTKMRM | 263 | CALR9p2 |
| RMMRTKWRM | 45 | RMMRTKMRM | 263 | CALR9p2 |
| RMMRTKMFM | 46 | RMMRTKMRM | 263 | CALR9p2 |
| MRMRRMRRL | 47 | MRMRRMRRT | 264 | CALR9p8 |
| MRMRRMRRM | 48 | MRMRRMRRT | 264 | CALR9p8 |
| MRMRRMRRI | 49 | MRMRRMRRT | 264 | CALR9p8 |
| MRMRRMRRV | 50 | MRMRRMRRT | 264 | CALR9p8 |
| MRMRRMRRF | 51 | MRMRRMRRT | 264 | CALR9p8 |
| MRMRRMRRY | 52 | MRMRRMRRT | 264 | CALR9p8 |
| MPMRRMRRT | 53 | MRMRRMRRT | 264 | CALR9p8 |
| MLMRRMRRT | 54 | MRMRRMRRT | 264 | CALR9p8 |
| MMMRRMRRT | 55 | MRMRRMRRT | 264 | CALR9p8 |
| MRMRRMRAT | 56 | MRMRRMRRT | 264 | CALR9p8 |
| MRMRRMRPT | 57 | MRMRRMRRT | 264 | CALR9p8 |
| MRMRRMRST | 58 | MRMRRMRRT | 264 | CALR9p8 |
| RRMRRMRRT | 59 | MRMRRMRRT | 264 | CALR9p8 |
| KMRRKMWPA | 60 | KMRRKMSPA | 265 | CALR9p19 |
| KMFRKMSPA | 61 | KMRRKMSPA | 265 | CALR9p19 |
| KMMRKMSPA | 62 | KMRRKMSPA | 265 | CALR9p19 |
| KMIRKMSPA | 63 | KMRRKMSPA | 265 | CALR9p19 |
| KMWRKMSPA | 64 | KMRRKMSPA | 265 | CALR9p19 |
| KMYRKMSPA | 65 | KMRRKMSPA | 265 | CALR9p19 |
| KMLRKMSPA | 66 | KMRRKMSPA | 265 | CALR9p19 |
| KMRRKMSPY | 67 | KMRRKMSPA | 265 | CALR9p19 |
| KMRRKMSPK | 68 | KMRRKMSPA | 265 | CALR9p19 |
| KPRRKMSPA | 69 | KMRRKMSPA | 265 | CALR9p19 |
| KMRRKMSPL | 70 | KMRRKMSPA | 265 | CALR9p19 |
| KMRRKMSPF | 71 | KMRRKMSPA | 265 | CALR9p19 |
| KMRRKMSPM | 72 | KMRRKMSPA | 265 | CALR9p19 |
| FMRRKMSPA | 73 | KMRRKMSPA | 265 | CALR9p19 |
| KMARKMSPA | 74 | KMRRKMSPA | 265 | CALR9p19 |
| KMVRKMSPA | 75 | KMRRKMSPA | 265 | CALR9p19 |
| KMRRKMSPR | 76 | KMRRKMSPA | 265 | CALR9p19 |
| KMRRKMSPW | 77 | KMRRKMSPA | 265 | CALR9p19 |
| YMRRKMSPA | 78 | KMRRKMSPA | 265 | CALR9p19 |
| KMREKMSPA | 79 | KMRRKMSPA | 265 | CALR9p19 |
| KMRDKMSPA | 80 | KMRRKMSPA | 265 | CALR9p19 |
| MMRRKMSPA | 81 | KMRRKMSPA | 265 | CALR9p19 |
| KMNRKMSPA | 82 | KMRRKMSPA | 265 | CALR9p19 |
| KMSRKMSPA | 83 | KMRRKMSPA | 265 | CALR9p19 |
| KMRRFMSPA | 84 | KMRRKMSPA | 265 | CALR9p19 |
| KMRRKMSPV | 85 | KMRRKMSPA | 265 | CALR9p19 |
| RPSCREACL | 86 | RTSCREACL | 266 | CALR9p30 |
| RTSCREACK | 87 | RTSCREACL | 266 | CALR9p30 |
| RTSCREACW | 88 | RTSCREACL | 266 | CALR9p30 |
| RESCREACL | 89 | RTSCREACL | 266 | CALR9p30 |
| RTKCREACL | 90 | RTSCREACL | 266 | CALR9p30 |
| RTRCREACL | 91 | RTSCREACL | 266 | CALR9p30 |
| RTSCRFACL | 92 | RTSCREACL | 266 | CALR9p30 |
| RTSCRHACL | 93 | RTSCREACL | 266 | CALR9p30 |
| RTSCRRACL | 94 | RTSCREACL | 266 | CALR9p30 |
| RTSCRWACL | 95 | RTSCREACL | 266 | CALR9p30 |
| RTSCRYACL | 96 | RTSCREACL | 266 | CALR9p30 |
| RTSCREACR | 97 | RTSCREACL | 266 | CALR9p30 |
| RQSCREACL | 98 | RTSCREACL | 266 | CALR9p30 |
| RLSCREACL | 99 | RTSCREACL | 266 | CALR9p30 |
| RMSCREACL | 100 | RTSCREACL | 266 | CALR9p30 |
| RTSCREACY | 101 | RTSCREACL | 266 | CALR9p30 |
| RYSCREACL | 102 | RTSCREACL | 266 | CALR9p30 |
| RRSCREACL | 103 | RTSCREACL | 266 | CALR9p30 |
| RPKMRMRRM | 104 | RTKMRMRRM | 267 | CALR9p5 |
| RTKMRMRAM | 105 | RTKMRMRRM | 267 | CALR9p5 |
| RTKMRMRPM | 106 | RTKMRMRRM | 267 | CALR9p5 |
| RTMMRMRRM | 107 | RTKMRMRRM | 267 | CALR9p5 |
| RTFMRMRRM | 108 | RTKMRMRRM | 267 | CALR9p5 |
| RTYMRMRRM | 109 | RTKMRMRRM | 267 | CALR9p5 |
| RTKMRMRRR | 110 | RTKMRMRRM | 267 | CALR9p5 |
| RTKMRMRRK | 111 | RTKMRMRRM | 267 | CALR9p5 |
| RTKMRMRRW | 112 | RTKMRMRRM | 267 | CALR9p5 |
| RTWMRMRRM | 113 | RTKMRMRRM | 267 | CALR9p5 |
| RTKMRMRRY | 114 | RTKMRMRRM | 267 | CALR9p5 |
| RQKMRMRRM | 115 | RTKMRMRRM | 267 | CALR9p5 |
| RRKMRMRRM | 116 | RTKMRMRRM | 267 | CALR9p5 |
| RTAMRMRRM | 117 | RTKMRMRRM | 267 | CALR9p5 |
| RTIMRMRRM | 118 | RTKMRMRRM | 267 | CALR9p5 |
| RTLMRMRRM | 119 | RTKMRMRRM | 267 | CALR9p5 |
| RTVMRMRRM | 120 | RTKMRMRRM | 267 | CALR9p5 |
| DTKMRMRRM | 121 | RTKMRMRRM | 267 | CALR9p5 |
| ETKMRMRRM | 122 | RTKMRMRRM | 267 | CALR9p5 |
| FTKMRMRRM | 123 | RTKMRMRRM | 267 | CALR9p5 |
| HTKMRMRRM | 124 | RTKMRMRRM | 267 | CALR9p5 |
| YTKMRMRRM | 125 | RTKMRMRRM | 267 | CALR9p5 |
| FRMRRTRRKM | 126 | RRMRRTRRKM | 268 | CALR10p11 |
| YRMRRTRRKM | 127 | RRMRRTRRKM | 268 | CALR10p11 |
| RPMRRTRRKM | 128 | RRMRRTRRKM | 268 | CALR10p11 |
| RRPRRTRRKM | 129 | RRMRRTRRKM | 268 | CALR10p11 |
| MRMRRTRRKM | 130 | RRMRRTRRKM | 268 | CALR10p11 |
| WRMRRTRRKM | 131 | RRMRRTRRKM | 268 | CALR10p11 |
| RLMRRTRRKM | 132 | RRMRRTRRKM | 268 | CALR10p11 |
| RMMRRTRRKM | 133 | RRMRRTRRKM | 268 | CALR10p11 |
| RRMRRTRRKR | 134 | RRMRRTRRKM | 268 | CALR10p11 |
| RQMRRTRRKM | 135 | RRMRRTRRKM | 268 | CALR10p11 |
| RSMRRTRRKM | 136 | RRMRRTRRKM | 268 | CALR10p11 |
| RTMRRTRRKM | 137 | RRMRRTRRKM | 268 | CALR10p11 |
| RYMRRTRRKM | 138 | RRMRRTRRKM | 268 | CALR10p11 |
| REMRRTRRKM | 139 | RRMRRTRRKM | 268 | CALR10p11 |
| RKMRRKMSPK | 140 | RKMRRKMSPA | 269 | CALR10p18 |
| RPMRRKMSPA | 141 | RKMRRKMSPA | 269 | CALR10p18 |
| RKPRRKMSPA | 142 | RKMRRKMSPA | 269 | CALR10p18 |
| RKMRRKMSPY | 143 | RKMRRKMSPA | 269 | CALR10p18 |
| RKMRRKMSPF | 144 | RKMRRKMSPA | 269 | CALR10p18 |
| RKMRRKMSPR | 145 | RKMRRKMSPA | 269 | CALR10p18 |
| RKMRRKMSPM | 146 | RKMRRKMSPA | 269 | CALR10p18 |
| RRMRRKMSPA | 147 | RKMRRKMSPA | 269 | CALR10p18 |
| RLMRRKMSPA | 148 | RKMRRKMSPA | 269 | CALR10p18 |
| RMMRRKMSPA | 149 | RKMRRKMSPA | 269 | CALR10p18 |
| RKMFRKMSPA | 150 | RKMRRKMSPA | 269 | CALR10p18 |
| RKMIRKMSPA | 151 | RKMRRKMSPA | 269 | CALR10p18 |
| RKMMRKMSPA | 152 | RKMRRKMSPA | 269 | CALR10p18 |
| RKMWRKMSPA | 153 | RKMRRKMSPA | 269 | CALR10p18 |
| RKMYRKMSPA | 154 | RKMRRKMSPA | 269 | CALR10p18 |
| RKMRRKMWPA | 155 | RKMRRKMSPA | 269 | CALR10p18 |
| REMRRKMSPA | 156 | RKMRRKMSPA | 269 | CALR10p18 |
| RKMRRKMSPL | 157 | RKMRRKMSPA | 269 | CALR10p18 |
| TKMRMRRMRK | 158 | TKMRMRRMRR | 270 | CALR10p6 |
| TKMYMRRMRR | 159 | TKMRMRRMRR | 270 | CALR10p6 |
| TTMRMRRMRR | 160 | TKMRMRRMRR | 270 | CALR10p6 |
| TVMRMRRMRR | 161 | TKMRMRRMRR | 270 | CALR10p6 |
| TIMRMRRMRR | 162 | TKMRMRRMRR | 270 | CALR10p6 |
| TKMRMRRMRL | 163 | TKMRMRRMRR | 270 | CALR10p6 |
| TKMRMRRMRF | 164 | TKMRMRRMRR | 270 | CALR10p6 |
| TKMRMRRMRI | 165 | TKMRMRRMRR | 270 | CALR10p6 |
| TKMRMRRMRM | 166 | TKMRMRRMRR | 270 | CALR10p6 |
| TKMRMRRMRV | 167 | TKMRMRRMRR | 270 | CALR10p6 |
| TAMRMRRMRR | 168 | TKMRMRRMRR | 270 | CALR10p6 |
| TSMRMRRMRR | 169 | TKMRMRRMRR | 270 | CALR10p6 |
| RKMRMRRMRR | 170 | TKMRMRRMRR | 270 | CALR10p6 |
| TRMRMRRMRR | 171 | TKMRMRRMRR | 270 | CALR10p6 |
| TMMRMRRMRR | 172 | TKMRMRRMRR | 270 | CALR10p6 |
| RPRRMRRTRRKM | 173 | RMRRMRRTRRKM | 271 | CALR12p9 |
| RMRRMRRTIRKM | 174 | RMRRMRRTRRKM | 271 | CALR12p9 |
| RMRRMRRTLRKM | 175 | RMRRMRRTRRKM | 271 | CALR12p9 |
| RMRRMRRTMRKM | 176 | RMRRMRRTRRKM | 271 | CALR12p9 |
| RMRRPRRTRRKM | 177 | RMRRMRRTRRKM | 271 | CALR12p9 |
| RMMRMRRTRRKM | 178 | RMRRMRRTRRKM | 271 | CALR12p9 |
| RMRRMRWTRRKM | 179 | RMRRMRRTRRKM | 271 | CALR12p9 |
| RMRRMRRWRRKM | 180 | RMRRMRRTRRKM | 271 | CALR12p9 |
| RMRRMRRTYRKM | 181 | RMRRMRRTRRKM | 271 | CALR12p9 |
| RMPRMRRTRRKM | 182 | RMRRMRRTRRKM | 271 | CALR12p9 |
| RMRPMRRTRRKM | 183 | RMRRMRRTRRKM | 271 | CALR12p9 |
| SPARPRTSCL | 184 | SPARPRTSCR | 272 | CALR10p25 |
| SPARPRTSCF | 185 | SPARPRTSCR | 272 | CALR10p25 |
| SPARPRTSCI | 186 | SPARPRTSCR | 272 | CALR10p25 |
| SPARPRTSCM | 187 | SPARPRTSCR | 272 | CALR10p25 |
| SPARPRTSCV | 188 | SPARPRTSCR | 272 | CALR10p25 |
| SPARPRTSCA | 189 | SPARPRTSCR | 272 | CALR10p25 |
| SPARPRTSIR | 190 | SPARPRTSCR | 272 | CALR10p25 |
| SPARPRTSLR | 191 | SPARPRTSCR | 272 | CALR10p25 |
| SPARPRTSMR | 192 | SPARPRTSCR | 272 | CALR10p25 |
| SPARPRTSVR | 193 | SPARPRTSCR | 272 | CALR10p25 |
| SFARPRTSCR | 194 | SPARPRTSCR | 272 | CALR10p25 |
| STARPRTSCR | 195 | SPARPRTSCR | 272 | CALR10p25 |
| SVARPRTSCR | 196 | SPARPRTSCR | 272 | CALR10p25 |
| SYARPRTSCR | 197 | SPARPRTSCR | 272 | CALR10p25 |
| SPARPRTSYR | 198 | SPARPRTSCR | 272 | CALR10p25 |
| SPARPRTWCR | 199 | SPARPRTSCR | 272 | CALR10p25 |
| SPARPRTSFR | 200 | SPARPRTSCR | 272 | CALR10p25 |
| SIARPRTSCR | 201 | SPARPRTSCR | 272 | CALR10p25 |
| SSARPRTSCR | 202 | SPARPRTSCR | 272 | CALR10p25 |
| SPAFPRTSCR | 203 | SPARPRTSCR | 272 | CALR10p25 |
| SAARPRTSCR | 204 | SPARPRTSCR | 272 | CALR10p25 |
| SPAYPRTSCR | 205 | SPARPRTSCR | 272 | CALR10p25 |
| SPARPRTSCK | 206 | SPARPRTSCR | 272 | CALR10p25 |
| SMARPRTSCR | 207 | SPARPRTSCR | 272 | CALR10p25 |
| SPFRPRTSCR | 208 | SPARPRTSCR | 272 | CALR10p25 |
| SPMRPRTSCR | 209 | SPARPRTSCR | 272 | CALR10p25 |
| SLARPRTSCR | 210 | SPARPRTSCR | 272 | CALR10p25 |
| SQARPRTSCR | 211 | SPARPRTSCR | 272 | CALR10p25 |
| SWARPRTSCR | 212 | SPARPRTSCR | 272 | CALR10p25 |
| SPAWPRTSCR | 213 | SPARPRTSCR | 272 | CALR10p25 |
| SPARPRTFCR | 214 | SPARPRTSCR | 272 | CALR10p25 |
| SPARPRTSCY | 215 | SPARPRTSCR | 272 | CALR10p25 |
| RTKMRMRMMR | 216 | RTKMRMRRMR | 273 | CALR10p5 |
| RTKMRMRRMK | 217 | RTKMRMRRMR | 273 | CALR10p5 |
| RTKMRMRFMR | 218 | RTKMRMRRMR | 273 | CALR10p5 |
| RTKMRMRLMR | 219 | RTKMRMRRMR | 273 | CALR10p5 |
| RTKMRMRWMR | 220 | RTKMRMRRMR | 273 | CALR10p5 |
| RTKMRMRYMR | 221 | RTKMRMRRMR | 273 | CALR10p5 |
| RTKMRMRIMR | 222 | RTKMRMRRMR | 273 | CALR10p5 |
| RTKMRMRVMR | 223 | RTKMRMRRMR | 273 | CALR10p5 |
| MTKMRMRRMR | 224 | RTKMRMRRMR | 273 | CALR10p5 |
| ETKMRMRRMR | 225 | RTKMRMRRMR | 273 | CALR10p5 |
| FTKMRMRRMR | 226 | RTKMRMRRMR | 273 | CALR10p5 |
| HTKMRMRRMR | 227 | RTKMRMRRMR | 273 | CALR10p5 |
| NTKMRMRRMR | 228 | RTKMRMRRMR | 273 | CALR10p5 |
| YTKMRMRRMR | 229 | RTKMRMRRMR | 273 | CALR10p5 |
| RTKMRMRRMW | 230 | RTKMRMRRMR | 273 | CALR10p5 |
| RTKMRMRRMY | 231 | RTKMRMRRMR | 273 | CALR10p5 |
| RTKMRMRRMF | 232 | RTKMRMRRMR | 273 | CALR10p5 |
| RTKMRMRRMM | 233 | RTKMRMRRMR | 273 | CALR10p5 |
| RTKMRMRRM1 | 234 | RTKMRMRRMR | 273 | CALR10p5 |
| RTKMRMRRML | 235 | RTKMRMRRMR | 273 | CALR10p5 |
| RTKMRMRRMV | 236 | RTKMRMRRMR | 273 | CALR10p5 |
| RRMRRTRRL | 237 | RRMRRTRRK | 274 | CALR9p11 |
| RRMRRTRRM | 238 | RRMRRTRRK | 274 | CALR9p11 |
| RRMRRTRRF | 239 | RRMRRTRRK | 274 | CALR9p11 |
| RIMRRTRRK | 240 | RRMRRTRRK | 274 | CALR9p11 |
| RMMRRTRRK | 241 | RRMRRTRRK | 274 | CALR9p11 |
| RTMRRTRRK | 242 | RRMRRTRRK | 274 | CALR9p11 |
| RVMRRTRRK | 243 | RRMRRTRRK | 274 | CALR9p11 |
| RSMRRTRRK | 244 | RRMRRTRRK | 274 | CALR9p11 |
| RLMRRTRRK | 245 | RRMRRTRRK | 274 | CALR9p11 |
| RAMRRTRRK | 246 | RRMRRTRRK | 274 | CALR9p11 |
| RQMRRTRRK | 247 | RRMRRTRRK | 274 | CALR9p11 |
| RFMRRTRRK | 248 | RRMRRTRRK | 274 | CALR9p11 |
| RWMRRTRRK | 249 | RRMRRTRRK | 274 | CALR9p11 |
| RYMRRTRRK | 250 | RRMRRTRRK | 274 | CALR9p11 |
| RCMRRTRRK | 251 | RRMRRTRRK | 274 | CALR9p11 |
| RGMRRTRRK | 252 | RRMRRTRRK | 274 | CALR9p11 |
| RNMRRTRRK | 253 | RRMRRTRRK | 274 | CALR9p11 |
| RRMRRTRFK | 254 | RRMRRTRRK | 274 | CALR9p11 |
| RRMRRTRYK | 255 | RRMRRTRRK | 274 | CALR9p11 |
| RRMRRTRRI | 256 | RRMRRTRRK | 274 | CALR9p11 |
| RRMRRTRRV | 257 | RRMRRTRRK | 274 | CALR9p11 |
| RRMRRTRRY | 258 | RRMRRTRRK | 274 | CALR9p11 |
| RRMRRTRRW | 259 | RRMRRTRRK | 274 | CALR9p11 |
| RRMRRTRRA | 260 | RRMRRTRRK | 274 | CALR9p11 |
| RRMRRTRRC | 261 | RRMRRTRRK | 274 | CALR9p11 |
| RRMRRTRRT | 262 | RRMRRTRRK | 274 | CALR9p11 |

TABLE B
Consensus Sequences

| | Heteroclitic | | Parental | Parental |
|---|---|---|---|---|
| Heteroclitic peptide | peptide | Parental peptide | peptide | peptide |
| consensus aa sequence | SEQ ID NO. | aa sequence | name | SEQ ID NO. |
| X1X2M3X4X5X6X7X8X9 (SEQ ID NO. 275) | 275 | RMMRTKMRM | CALR9p2 | 263 |
| wherein: | | | | |
| X1 is: R, Y, F, M, or W | | | | |
| X2 is: M, Y, P, S, T, A, E, R, Q, F, or W | | | | |
| X4 is: R, D, or E | | | | |
| X5 is: T, W, Y, H, K, R, or F | | | | |
| X6 is: K, F, I, L, V, M, W, Y, T, C, N, or S | | | | |
| X7 is: M, or W | | | | |
| X8 is: R, A, P, S, Y, or F | | | | |
| X9 is: M, K, V, F, R, Y, W, or H | | | | |
| X1X2M3R4R5M6R7X8X9 (SEQ ID NO. 276) | 276 | MRMRRMRRT | CALR9p8 | 264 |
| Wherein: | | | | |
| X1 is: M, or R | | | | |
| X2 is: R, P, L, or M | | | | |
| X8 is: R, A, P, or S | | | | |
| X9 is: T, L, M, I, V, F, or Y | | | | |
| X1X2X3X4X5M6X7P8X9 (SEQ ID NO. 277) | 277 | KMRRKMSPA | CALR9p19 | 265 |
| Wherein: | | | | |
| X1 is: K, F, Y, or M | | | | |
| X2 is: M, or P | | | | |
| X3 is: R, F, M, I, W, Y, L, A, V, N, or S | | | | |
| X4 is: R, E, or D | | | | |
| X5 is: K, or F | | | | |
| X7 is: S, or W | | | | |
| X9 is: A, Y, K, L, F, M, R, W, or V | | | | |
| R1X2X3C4R5X6A7C8X9 (SEQ ID NO. 278) | 278 | RTSCREACL | CALR9p30 | 266 |
| Wherein: | | | | |
| X2 is: T, P, E, Q, L, M, Y, or R | | | | |
| X3 is: S, K, or R | | | | |
| X6 is: E, F, H, R, W, or Y | | | | |
| X9 is: L, K, W, R, or Y | | | | |
| X1X2X3M4R5M6R7X8X9 (SEQ ID NO. 279) | 279 | RTKMRMRRM | CALR9p5 | 267 |
| Wherein: | | | | |
| X1 is: R, D, E, F, H, or Y | | | | |
| X2 is: T, P, Q, or R | | | | |
| X3 is: K, M, F, Y, W, A, I, L, or V | | | | |
| X8 is: R, A, or P | | | | |
| X9 is: M, R, K, W, or Y | | | | |
| X1X2X3R4R5T6R7R8K9X10(SEQ ID NO. 280) | 280 | RRMRRTRRKM | CALR10p11 | 268 |
| Wherein: | | | | |
| X1 is: R, F, Y, M, or W | | | | |
| X2 is: R, P, L, M, Q, S, T, Y, or E | | | | |
| X3 is: M, or P | | | | |
| X10 is: M, or R | | | | |
| R1X2X3X4R5K6M7X8P9X10 (SEQ ID NO. 281) | 281 | RKMRRKMSPA | CALR10p18 | 269 |
| Wherein: | | | | |
| X2 is: K, P, R, L, M, or E | | | | |
| X3 is: M, or P | | | | |
| X4 is: R, F, I, M, W, or Y | | | | |
| X8 is: S, or W | | | | |
| X10 is: A, K, Y, F, R, M, or L | | | | |
| X1X2M3X4M5R6R7M8R9X10 (SEQ ID NO. 282) | 282 | TKMRMRRMRR | CALR10p6 | 270 |
| Wherein: | | | | |
| X1 is: T, or R | | | | |
| X2 is: K, T, V, I, A, S, R, or M | | | | |
| X4 is: R, or Y | | | | |
| X10 is: R, K, L, F, I, M, or V | | | | |
| R1X2X3X4X5R6X7X8X9R10K11M12 (SEQ ID NO. | 283 | RMRRMRRTRRKM | CALR12p9 | 271 |
| 283) | | | | |
| Wherein: | | | | |
| X2 is: M, or P | | | | |
| X3 is: R, M, or P | | | | |
| X4 is: R, or P | | | | |
| X5 is: M, or P | | | | |
| X7 is: R, or W | | | | |
| X8 is: T, or W | | | | |
| X9 is: R, I, L, M, or Y | | | | |
| S1X2X3X4P5R6T7X8X9X10 (SEQ ID NO. 284) | 284 | SPARPRTSCR | CALR10p25 | 272 |
| Wherein: | | | | |
| X2 is: P, F, T, V, Y, I, S, A, M, L, Q, or W | | | | |
| X3 is: A, F, or M | | | | |
| X4 is: R, F, Y, or W | | | | |
| X8 is: S, W, or F | | | | |
| X9 is: C, I, L, M, V, Y, or F | | | | |
| X10 is: R, L, F, I, M, V, A, K, or Y | | | | |
| X1T2K3M4R5M6R7X8M9X10 (SEQ ID NO. 285) | 285 | RTKMRMRRMR | CALR10p5 | 273 |
| Wherein: | | | | |
| X1 is: R, M, E, F, H, N, or Y | | | | |
| X8 is: R, M, F, L, W, Y, I, or V | | | | |
| X10 is: R, K, W, Y, F, M, I, L, or V | | | | |
| R1X2M3R4R5T6R7X8X9 (SEQ ID NO. 286) | 286 | RRMRRTRRK | CALR9p11 | 274 |
| Wherein: | | | | |
| X2 is: R, I, M, T, V, S, L, A, | | | | |
| Q, F, W, Y, C, G, or N | | | | |
| X8 is: R, F, or Y | | | | |
| X9 is: K, L, M, F, I, V, Y, W, A, C, or T | | | | |

In some embodiments, the heteroclitic CALR^{MUT} peptides described herein have one or more the following superior properties: (a) superior immunogenicity as compared to their native counterparts, (b) superior HLA binding (e.g. affinity) as compared to their native counterparts, (c) an HLA-I binding affinity of <500 nm, (d) an HLA-I binding affinity of <100 nm, (d) being a superior T cell receptor (TCR) epitope as compared to their native counterparts, (e) superior (e.g., increased) TCR agonist activity as compared to their native counterparts, (f) superior induction of CD8+ T cell responses as compared to their native counterparts, (g) induction of superior (e.g. increased) antigen-specific (i.e. CALRMUT-specific) antitumor immunity as compared to their native counterparts.

In certain embodiments the present invention also provides variants of the heteroclitic CALR^{MUT} peptides described herein. In some embodiments such variants comprise 1 or 2 or 3 or more amino acid point mutations as compared to any of SEQ ID Nos 1-262, or have an amino acid sequence that is at least 90%, or at least 95%, or at least 96%, or at least 97%, or at least 98%, or at least 99% identical to any of SEQ ID Nos 1-262, provided that such variants are heteroclitic, and/or exhibit one or more the superior properties described above.

In some embodiments a heteroclitic CALR^{MUT} peptide as described herein is 8, or 9, or 10, or 11, or 12, or 13 amino acids in length. In some embodiments a heteroclitic CALR^{MUT} peptide as described herein is 8-13 amino acids in length. In some embodiments a heteroclitic CALR^{MUT} peptide as described herein is 9-12 amino acids in length.

In some embodiments a heteroclitic CALR^{MUT} peptide as described herein comprises one amino acid point mutation as compared to the parental peptide from which it is derived. In some embodiments a heteroclitic CALR^{MUT} peptide as described herein comprises two amino acid point mutations as compared to the parental peptide from which it is derived. In some embodiments, where the heteroclitic CALR^{MUT} peptides comprise two amino acid point mutations, those mutations can be a combination of any two of the single amino acid point mutations described herein (e.g. the single point mutations present in SEQ ID Nos. 1-262).

### Nucleic Acid Molecules

In some embodiments the present invention provides nucleic acid molecules that encode the heteroclitic CALR^{MUT} peptides described herein. In some embodiments the nucleic acid molecules are DNA. In some embodiments the nucleic acid molecules are RNA. In some embodiments the nucleic acid molecules are mRNA. All such nucleic acid molecules can comprise naturally occurring nucleotides or synthetic and/or chemically modified nucleotides—such as those that are modified to increase their stability or otherwise improve their suitability for administration to subjects.

### Vectors

In some embodiments the present invention provides “vectors” that comprise nucleic acid molecules that encode the heteroclitic CALR^{MUT} peptides described herein.

The term “vector,” as used herein, means a construct suitable for delivery of a nucleic acid molecule to a cell. Examples of vectors include, but are not limited to, viruses, viral-derived vectors, naked DNA or RNA vectors, plasmid vectors, cosmid vectors, phage vectors, and the like. In some embodiments a vector may be an “expression vector” that is capable of delivering a nucleic acid molecule to a cell and that also contains elements required for expression of the nucleic acid molecule in the cell.

In some embodiments the vectors are viral vectors. Examples of suitable viral vectors include, but are not limited to, adenoviral vectors, adeno-associated virus vectors, retroviral vectors, lentiviral vectors, alphavirus vectors, and vaccinia virus vectors.

### Compositions

The present invention provides various compositions comprising the heteroclitic CALR^{MUT} peptides, nucleic acid molecules, or vectors described herein.

In some embodiments the compositions described herein comprise one or more additional components suitable for administration to a subject and/or useful in formulating a composition for delivery to a subject, including, but not limited to, diluents, buffers, carriers, stabilizers, dispersing agents, suspending agents, thickening agents, excipients, preservatives, and the like.

In some embodiments, the compositions described herein also comprise an adjuvant. Adjuvants are well known in the art and any suitable adjuvant can be used. Examples of adjuvants that can be used in the compositions and methods of the present invention include, but are not limited to: inorganic or organic adjuvants, oil-based adjuvants, virosomes, liposomes, lipopolysaccharide (LPS), monophosphoryl lipid A (MPL), saponin, saponin QS-21, CpG oligonucleotides, molecular cages for antigens (such as immune-stimulating complexes (“ISCOMS”)), Ag-modified saponin/cholesterol micelles that form stable cage-like structures that are transported to the draining lymph nodes), components of bacterial cell walls, nucleic acids (such as double-stranded RNA (dsRNA), single-stranded DNA (ssDNA), and unmethylated CpG dinucleotide-containing DNA), alum, aluminum phosphate, aluminum hydroxide, squalene, Freund's Complete Adjuvant, Freund's Incomplete Adjuvant, and the like.

### Delivery Vehicles

In some embodiments the compositions of the present invention comprise a delivery vehicle. The term “delivery vehicle,” as used herein, refers to a substance useful for the delivery of either a nucleic acid molecule or a peptide to a cell. Examples of delivery vehicles that can be used in conjunction with the present invention include, but are not limited to, nanoparticles, lipid nanoparticles, liposomes, lipids, lipid encapsulation systems, polymers, and polymersomes.

### Methods of Treatment

The present invention provides various methods of treatment. For example, in some embodiments the present invention provides methods of treating JAK2 mutant-negative myeloproliferative neoplasms (MPNs) in subjects in need thereof, such methods comprising administering to a subject an effective amount of a heteroclitic CALR^{MUT} peptide, nucleic acid molecule, vector or composition as described herein. In some embodiments such methods involve administering one dose of a heteroclitic CALR^{MUT} peptide, nucleic acid molecule, vector or composition to the subject. In some embodiments such methods involve administering two or more doses of a heteroclitic CALR^{MUT} peptide, nucleic acid molecule, vector or composition to the subject. For example, in some embodiments such treatment methods involve administering a priming dose and one or more booster doses of the heteroclitic CALR^{MUT} peptide, nucleic acid molecule, vector or composition to the subject. In some embodiments such methods also comprise administering an effective amount of an immune checkpoint inhibitor to the subject. Suitable immune checkpoint inhibitors include PD-1, PD-L1, PD-L2 and CTLA-4 inhibitors. In some embodiments the immune checkpoint inhibitor is an anti-PD1 antibody.

As used herein, the terms “treat,” “treating,” and “treatment” refer achieving, and/or administering an agent or agents (e.g., a heteroclitic CALR^{MUT} peptide, nucleic acid molecule, vector or composition as described herein) to a subject to achieve, to a detectable degree, an improvement in one or more clinically relevant parameters in a subject (e.g., a subject with a JAK2 mutant-negative MPN), or in a cancer/tumor (e.g. a JAK2 mutant-negative MPN), or in tumor cells (e.g., JAK2 mutant-negative JAK2 mutant-negative MPN tumor cells). Such clinically relevant parameters include, but are not limited to, reducing the rate of growth of a tumor (or tumor cells), halting the growth of a tumor (or of tumor cells), causing regression of a tumor (or of tumor cells), reducing the size of a tumor (for example as measured in terms of tumor volume or tumor mass), reducing the grade of a tumor, eliminating a tumor (or tumor cells), preventing, delaying, or slowing recurrence (rebound) of a cancer/tumor, improving symptoms associated with a cancer/tumor, improving survival from a cancer/tumor, inhibiting or reducing spreading of a cancer/tumor (e.g., metastases), and the like. Importantly, in the context of the present invention, such clinically relevant parameters also include (a) an immune response to a tumor or tumor cells, (b) a CD8+ T cell response to a tumor or tumor cells, (c) an anti-CALR^{MUT} immune response, (d) an anti-CALR^{MUT} CD8+ T cell response, and (e) enhanced sensitivity of a tumor or tumor cells to immune checkpoint blockade. All of the above are desirable biological outcomes of the present methods. In some embodiments, the improvement in the one or more clinically relevant parameters is assessed in comparison to a suitable baseline or suitable control. For example, in some embodiments the improvement in the one or more clinically relevant parameters is assessed in comparison to the level/extent of that clinically relevant parameter in the same subject prior to that subject being treated with a heteroclitic CALR^{MUT} peptide, nucleic acid molecule, vector or composition as described herein. Similarly, in some embodiments the improvement in the one or more clinically relevant parameters is assessed in comparison to the level/extent of that clinically relevant parameter in a suitable control subject or group of control subjects not treated with a heteroclitic CALR^{MUT} peptide, nucleic acid molecule, vector or composition as described herein (e.g., in a subject or group or group of subjects treated with a placebo). In some embodiments the improvement in the one or more clinically relevant parameters is a statistically significant improvement.

In some embodiments the present methods and compositions can be used to treat any JAK2 mutant-negative MPN in a subject in need thereof.

In some embodiments the subject has a tumor that is resistant to treatment using other methodologies and/or compositions. As used herein, the terms “resistant” and “resistance” are used consistent with their normal usage in the art and consistent with the understanding of the term by physicians who treat cancer. For example, consistent with its usual meaning in the art, a tumor or a subject may be considered “resistant” to a certain treatment method or treatment with a certain agent (or combination of agents), if, despite using that method or administering that agent (or combination of agents), a subject's tumor (or tumor cells) grows, and/or progresses, and/or spreads, and/or metastasizes, and/or recurs. In some instances, a tumor may initially be sensitive to treatment with a certain method or agent (or combination of agents), but later became resistant to such treatment.

As used herein the term “subject” encompasses all mammalian species, including, but not limited to, humans, non-human primates, dogs, cats, rodents (such as rats, mice and guinea pigs), cows, pigs, sheep, goats, horses, and the like—including all mammalian animal species used in animal husbandry, as well as animals kept as pets and in zoos, etc. In preferred embodiments the subjects are human.

In some embodiments the subject has a JAK2 mutant-negative MPN. In some such embodiments the subject has a JAK2^{V617F} mutant-negative MPN. In some embodiments the subject has a JAK2 mutant-negative MPN that has recurred following a prior treatment with other compositions or methods, including, but not limited to, chemotherapy, radiation therapy, or surgical resection, or any combination thereof. In some embodiments the subject has a JAK2 mutant-negative MPN that has not previously been treated.

As used herein the term “effective amount” refers to an amount of an active agent (e.g., a heteroclitic CALR^{MUT} peptide, nucleic acid molecule, vector or composition) as described herein that is sufficient to achieve, or contribute towards achieving, one or more desirable clinical outcomes, such as those described in the “treatment” description above. An appropriate “effective amount” in any individual case may be determined using standard techniques known in the art, such as dose escalation studies, and may be determined taking into account such factors as the desired route of administration (e.g., systemic vs. intratumoral), desired frequency of dosing, and patient characteristics such as a subject's age, sex, body weight, etc. Furthermore, an “effective amount” may be determined in the context of any co-administration method to be used. One of skill in the art can readily perform such dosing studies (whether using single agents or combinations of agents) to determine appropriate doses to use, for example using assays such as those described in the Examples section of this patent application—which involve administration of the agents described herein to subjects (such as animal subjects routinely used in the pharmaceutical sciences for performing dosing studies). For example, in some embodiments an “effective amount” an active agent (e.g., a heteroclitic CALR^{MUT} peptide, nucleic acid molecule, vector or composition) as described herein may be calculated based on studies in humans or other mammals carried out to determine efficacy of the active agent.

In some embodiments one or more of the active agents (e.g., a heteroclitic CALR^{MUT} peptide, nucleic acid molecule, vector or composition) described herein is used at approximately its maximum tolerated dose, for example as determined in phase I clinical trials and/or in dose escalation studies. In some embodiments one or more of the active agents is used at about 90% of its maximum tolerated dose. In some embodiments one or more of the active agents is used at about 80% of its maximum tolerated dose. In some embodiments one or more of the active agents is used at about 70% of its maximum tolerated dose. In some embodiments one or more of the active agents is used at about 60% of its maximum tolerated dose. In some embodiments one or more of the active agents is used at about 50% of its maximum tolerated dose. In some embodiments one or more of the active agents is used at about 50% of its maximum tolerated dose. In some embodiments one or more of the active agents is used at about 40% of its maximum tolerated dose. In some embodiments one or more of the active agents is used at about 30% of its maximum tolerated dose.

In carrying out the treatment methods described herein, any suitable method or route of administration can be used to deliver the active agents described herein. In some embodiments systemic administration may be employed, for example, oral or intravenous administration, or any other suitable method or route of systemic administration known in the art. In some embodiments intratumoral delivery may be employed. For example, the active agents described herein may be administered either systemically or locally by injection, by infusion through a catheter, using an implantable drug delivery device, or by any other means known in the art. One of skill in the art will be able to select the appropriate delivery method or route depending on the situation, for example depending on whether active agents or cells are being administered, and in the case of active agents, depending on the nature of the active agent (e.g., its stability, half-life, etc.).

In certain embodiments the compositions and methods of treatment provided herein may be employed together with other compositions and treatment methods known to be useful for tumor therapy, including, but not limited to, surgical methods (e.g., for tumor resection), radiation therapy methods, treatment with chemotherapeutic agents, treatment with antiangiogenic agents, treatment with tyrosine kinase inhibitors or treatment with immune checkpoint inhibitors. Similarly, in certain embodiments the methods of treatment provided herein may be employed together with procedures used to monitor disease status/progression, such as biopsy methods and diagnostic methods (e.g., MRI methods or other imaging methods).

For example, in some embodiments the methods described herein may be performed prior to performing surgical resection of a tumor, for example in order to shrink a tumor prior to surgical resection. In other embodiments the methods described herein may be performed both before and after performing surgical resection of a tumor.

In some embodiments the treatment methods described herein may be employed in conjunction with performing a diagnostic test to determine if the subject has a tumor that that is likely to be responsive to therapy. For example, in some embodiments, the treatment methods provided herein comprise performing a diagnostic test to determine if the subject has a JAK2 mutant-negative MPN. Typically, such a test will be performed prior to administering one or more of the active agents (e.g., heteroclitic CALR^{MUT} peptides, nucleic acid molecules, vectors or compositions) described herein.

The invention is further described in the following non-limiting Examples.

### EXAMPLES

Numbers in parentheses in these Examples refer to the numbered references in the Reference List that follows this Examples section.

### Example 1

### Overview

The majority of JAK2^{V617F}-negative myeloproliferative neoplasms (MPN) have disease-initiating frameshift mutations in calreticulin (CALR) resulting in a common novel C-terminal mutant fragment (CALRMUT), representing an attractive source of neoantigens for cancer vaccines. However, studies have shown that CALRMUT-specific T cells are rare in CALRMUT MPN patients. The underlying reasons for this phenomenon are unknown. In this study, we examined class-I major histocompatibility complex (MHC-I) allele frequency in CALRMUT MPN patients from two independent cohorts and observed that MHC-I alleles that present CALRMUT neoepitopes with high affinity are under-represented in CALRMUT MPN patients. We believe that this is due to an increased chance of immune-mediated tumor rejection by individuals expressing one of these MHC-I alleles such that the disease never clinically manifests. As a consequence of this MHC-I allele restriction, we hypothesized that CALRMUT MPN patients might not efficiently respond to cancer vaccines composed of the CALRMUT fragment, but might do so when immunized with a properly modified CALRMUT heteroclitic peptide vaccine approach. We found that heteroclitic CALRMUT peptides specifically designed for CALRMUT MPN patient MHC-I alleles efficiently elicited a cross-reactive CD8+ T cell response in human PBMC samples otherwise unable to respond to the matched weakly immunogenic CALRMUT native peptides. We also modeled this effect in mice and observed that C57BL/6J mice, which are unable to mount an immune response to the human CALRMUT fragment, can mount a cross-reactive CD8+ T cell response against a CALRMUT-derived peptide upon heteroclitic peptide immunization and this was further amplified by combining the heteroclitic peptide vaccine with blockade of the immune checkpoint molecule PD-1. Together, our data demonstrate the therapeutic potential of heteroclitic peptide-based cancer vaccines in CALRMUT MPN patients.

### INTRODUCTION

Philadelphia chromosome-negative myeloproliferative neoplasms (MPNs) are myeloid blood cancers arising from hematopoietic stem cells (1, 2) and are characterized by hyperactivated JAK-STAT signaling (3). The majority of JAK2^{V617F}-negative MPN tumors have an insertion or deletion (INDEL) mutation in the C-terminal region of calreticulin (CALR) creating a +1 base-pair frameshift (4, 5). While multiple unique INDELs are found, nearly all generate a 44 amino acid common peptide, although a few rare cases generate a shorter 36 amino acid fragment (4, 5). Mutant CALR (CALRMUT) develops a pathogenic binding interaction with the extracellular portion of the thrombopoietin receptor (MPL), inducing ligand-independent constitutive JAK-STAT signaling pathways activation and oncogenesis (6-8). Consequently, the oncogenic CALRMUT fragment is an attractive source of mutational frameshift neoantigens for cancer vaccines in CALRMUT-positive MPN patients (9). However, in the few studies examining CALRMUT fragment immunogenicity, T cells from CALRMUT MPN patients had less immunoreactivity to CALRMUT-derived peptides compared to healthy individuals (10-14), even though many immunogenic peptides are predicted (14, 15). Interestingly, T cells from healthy donors display a stronger and more frequent response to CALRMUT peptides compared to T cells from patients with CALRMUT MPN (13). Additionally, several of the healthy donor T cell responses were elicited by memory T cells (13). This indicates that CALRMUT peptides are immunogenic in normal donors and suggest that CALRMUT-specific immune responses may be a mechanism of immunosurveillance eliminating the early tumor before its clinical manifestation. However, it is not clear how the tumor could escape this level of control in patients with clinical disease and why T cells from these patients did not respond to the CALRMUT fragment.

For antigens to be recognized by CD8+ T cells they must first be processed into smaller peptides, translocate to the endoplasmic reticulum (ER) and, if conditions are met, bind to the class I major histocompatibility complex (MHC-I) to form a peptide:MHC-I complex (pMHC-I) capable of reaching the cell surface to be recognized by the T cell receptor (TCR). However, not all peptides fit the stoichiometric requirements for MHC-I binding. Successful pMHC-I binding requires peptides to be the correct length and to have the appropriate anchor residues at specific locations to stabilize binding to pockets of the MHC-I allele (16). However, MHC genes have evolved to be extremely polymorphic across populations. Humans encode two copies of three different MHC-I genes, human leukocytes antigens (HLA)-A, HLA-B and HLA-C, with each having hundreds or thousands of polymorphisms (17). Importantly, different polymorphic residues alter the anchor residues such that peptides that bind to some MHC-I alleles may not bind to others (18, 19). As a result, the sum of presented peptides can vary greatly across individuals. We therefore hypothesized that some individuals possess the ability to present CALRMUT-derived peptides and eliminate early CALRMUT-positive MPNs, while other individuals do not and are more likely to develop the disease.

Since many peptides possess some but not all stoichiometric requirements for pMHC-I binding, affinities can range from strong to weak with many intermediate affinities possible. This affinity affects the number of surface-bound pMHC-I available for recognition by CD8+ T cells (20) and ultimately their activation. Notably, naïve T cells require higher levels of antigen stimulation than antigen-experienced T cells to potentiate T cell activation (21-26) and some antigens may be unable to activate naïve T cells. However, heteroclitic peptides (also known as anchor-optimized or anchor-improved peptides) are peptides in which one or two residues are specifically altered in order to increase MHC binding affinity. These resulting strong MHC-binding peptides robustly activate naïve T cells, and are similar enough to the original peptides that the activated T cells cross react with the original antigen (27-30), assuming that the original antigen has an adequate intermediate binding affinity.

In this study, we investigated two independent MPN patient cohorts and found that six MHC-I alleles predicted to efficiently bind to multiple CALRMUT-derived peptides were less frequently observed in CALRMUT MPN patients. This strongly pointed to a higher risk of developing CALRMUT MPN in patients lacking these MHC-I alleles and, at the same time, suggested to us that individuals with these MHC-I alleles could potentially control primordial CALRMUT-expressing tumors as part of the immunoediting process. In addition, this suggested to us that CALRMUT positive MPN patients are unlikely to respond to cancer vaccines composed of the CALRMUT fragment. Therefore, we analyzed the CALRMUT fragment for peptides that could be modified into heteroclitic peptides and serve as more potent anti-CALRMUT vaccines. We first tested this approach in in vitro assays using peripheral blood mononuclear cells (PBMCs) from healthy donors unable to respond to CALRMUT peptides and found that the same T cells could be induced to release IFNγ when primed using heteroclitic peptides. Then, to verify whether heteroclitic CALRMUT peptides can control the growth of CALRMUT tumors in vivo, we tested them as a vaccine in a pre-clinical mouse model. We established that C57BL/6J mice, which were unable to mount an immune response against the original CALRMUT fragment, had significantly delayed tumor growth when given a heteroclitic peptide vaccine of the same specificity and that this was further enhanced by PD1 blockade.

### Results & Discussion

### CALRMUT MPN Patients Demonstrate Skewed MHC Allele Frequencies

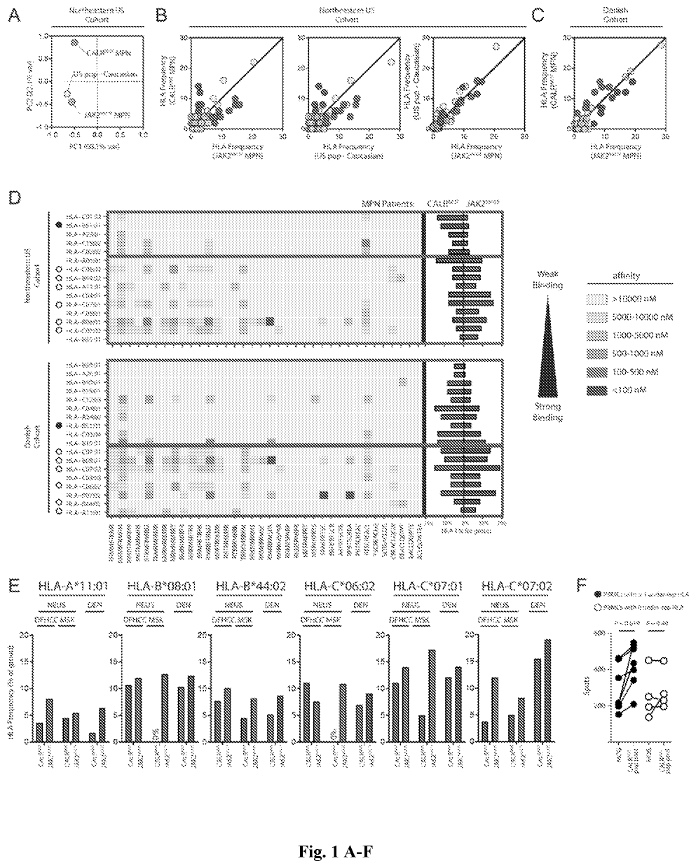

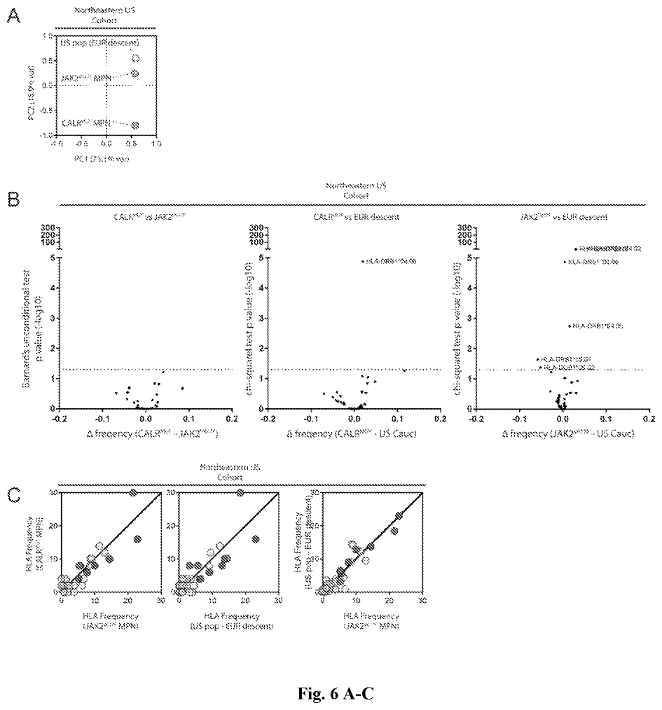

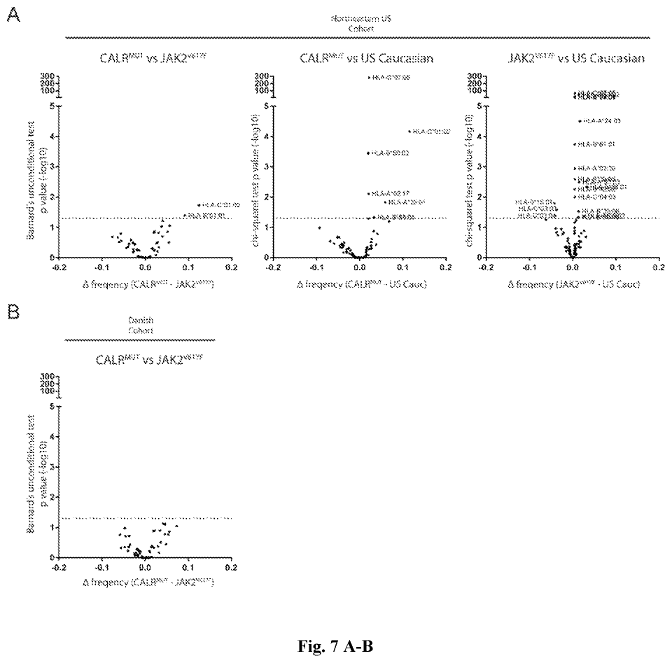

We investigated MHC-I and MHC-II allele frequencies in CALRMUT and JAK2^{V617F} MPN patients using haplotypes collected from two medical centers in the Northeastern United States (NEUS). In parallel, we assessed MHC-I allele frequencies of patients with MPN from eight medical centers in Denmark in order to independently validate the results observed in the NEUS cohort. MHC-II haplotypes were unavailable for the Danish cohort. As MHC allele frequencies vary greatly by geographic location (31), we analyzed each cohort separately. Furthermore, since the NEUS cohort is 88% Caucasian, we also compared MHC-I and MHC-II allele frequencies to those found in the US Caucasian population from the National Marrow Donor Program (17). To test for MHC-I and MHC-II allele frequency differences, we performed a principal component analysis comparing MHC-I and MHC-II allele frequencies from both NEUS MPN groups and the general US Caucasian population. We observed that the JAK2^{V617F} MPN group clusters in proximity to the US Caucasian group, while the CALRMUT MPN group is isolated for both MHC-I (FIG. 1A) and MHC-II (FIG. 6) allele frequencies, suggesting distinct MHC-I and MHC-II allele representation in the CALRMUT MPN patients. We then examined the MHC-I alleles with frequencies that were different in CALRMUT MPN patients compared to both JAK2^{V617F} MPN patients and the US Caucasian population in the NEUS cohort (FIG. 1B), and likewise those in CALRMUT MPN patients compared to the JAK2^{V617F} MPN patients from the Danish cohort (FIG. 1C). No single MHC-I allele reached statistical significance in both cohorts using Barnard's unconditional test (used for moderate numbers) or the chi-square test (FIG. 7A,B), and we, therefore, opted to analyze alleles with a fold change above or below a set threshold of ±0.2 fold frequency change in the NEUS cohort and ±0.125 fold frequency change in the Danish cohort (FIG. 1B,C). We observed that in CALRMUT MPN patients from both cohorts only HLA-B*51:01 is over-represented, while six MHC-I alleles are under-represented: HLA-A*11:01, HLA-B*08:01, HLA-B*44:02, HLA-C*07:01, HLA-C*07:02 and HLA-C*06:02, and this trend is also reflected in the fraction of patients which are positive for these alleles. For HLA-II alleles, no single allele had a statistically significant decrease in frequency in CALRMUT MPN patients (FIG. 6B) and the same fold-change threshold approach as for HLA-I alleles was therefore applied. We observed that HLA-DRB1*03:01, HLA-DRB1*04:01, HLA-DRB1*07:01, HLA-DRB1*13:01, HLA-DQB1*02:01 and HLA-DQB1*06:03 were less frequent in CALRMUT MPN patients compared to JAK2^{V617F} MPN patients and US individuals of European descent, while HLA-DRB1*11:01 and HLA-DRB1*11:04 were more frequent (FIG. 6C). Collectively our data suggest that CALRMUT MPN patients have skewed MHC-I and MHC-II haplotypes, whereas this does not appear to be the case for JAK2^{V617F} MPN patients.

Skewing of MHC-I Allele Frequencies is Associated with CALRMUT Peptide Binding Affinity

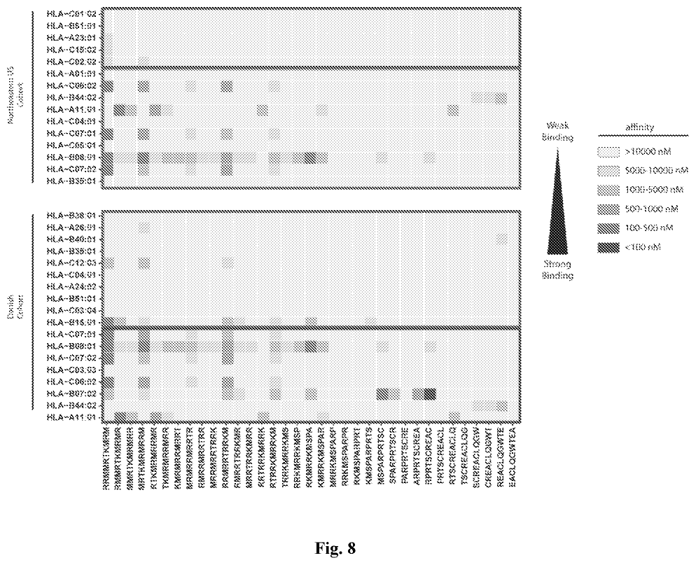

To determine if there are any correlations between MHC-I and MHC-II allele frequency skewing to the binding of the peptides derived from the 44 amino-acid mutant protein fragment, we compared the predicted binding affinity of the CALRMUT-derived peptides to each MHC-I and MHC-II allele with over- or under-represented frequencies using NetMHCpan 3.0 and NetMHCIIpan 3.2, respectively. Five of the six under-represented MHC-I alleles (except HLA-B*44:02) had a moderate predicted affinity (<10000 nM) to approximately a quarter of all 9-mer peptides of which many had <500 nM predicted affinity (FIG. 1D). In addition, multiple 10-mer peptides also had a high predicted affinity to these MHC-I alleles (FIG. 8). On the other hand, all MHC-I alleles that were over-represented in CALRMUT MPN patients had poor binding to almost all CALRMUT-derived peptides with the exception of HLA-B*15:01 and HLA-C*12:03 which only had a high frequency in the Danish cohort (FIG. 1D). When the NEUS cohort was separated in the two original cohorts (Memorial Sloan Kettering (MSK) and Dana Farber Harvard Cancer Center (DFHCC)) the same MHC-I skewing was confirmed except for HLA-C*06:02 which was slightly elevated in patients from MSK (FIG. 1E). To test whether these six under-represented MHC-I alleles were more likely to potentiate an immune response against the CALRMUT fragment, PBMCs from 7 healthy donors positive for at least one of the under-represented MHC-I alleles and PBMCs from 4 healthy donors that were negative for these MHC-I alleles were stimulated in vitro with peptides covering the entire CALRMUT fragment and examined for reactivity by IFNγ ELISpot following a final peptide restimulation. In the patients positive for the under-represented MHC-I alleles, 7/7 (100%) responded while only ¼ (25%) of patients negative for the MHC-I alleles (FIG. 1F). Therefore, while we did not test each MHC-I allele individually, we can conclude that, as a group, these six under-represented MHC-I alleles can potentiate an immune response against the CALRMUT fragment.

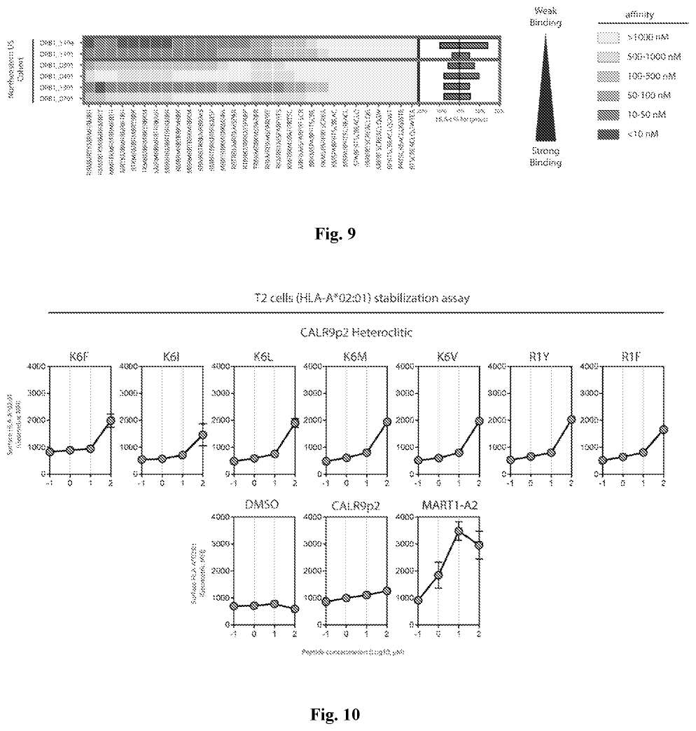

We did not observe the same trend for MHC-II alleles. Both MHC-II alleles found at higher frequency in CALRMUT MPN patients were predicted to bind strongly to more than half of 15-mer peptides, whereas only one of the four MHC-II alleles found at lower frequency appears to do so (FIG. 9). Interestingly, when we examined the MHC-II alleles of the healthy donors from which PBMCs were used to generate memory CD4+ T cell T cell lines against long CALRMUT peptides in a previous study (13), both donors were positive for HLA-DRB1*13:01 and one of these was further positive for HLA-DRB1*04:01.

Thus, here we show that patients with CALRMUT-positive MPNs were less likely to possess an MHC-I allele predicted to bind to peptides derived from the CALRMUT fragment. This may be due to the fact that individuals with MHC-I alleles that can bind to CALR-derived peptides are less likely to develop CALRMUT MPN.

### MHC-I Skewing in CALRMUT MPN is Specific for CALRMUT-Derived Peptides

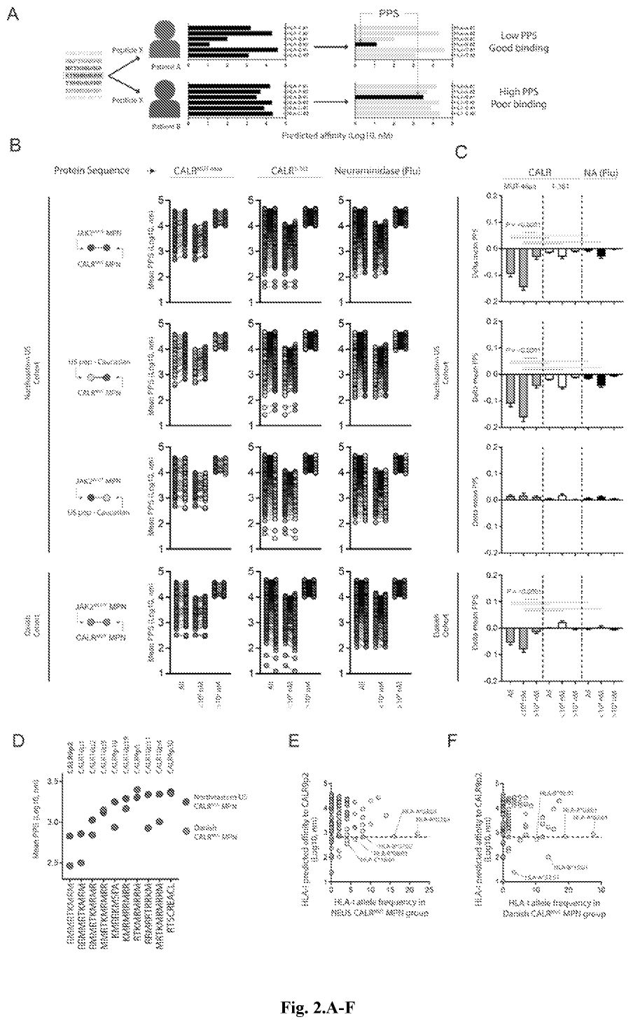

Prediction algorithms for pMHC-I binding based on neural networks like NetMHC are generally accepted to be useful yet imperfect tools, and their biases are typically hard to capture. To control for the prediction algorithm, we hypothesized that MHC-I allele frequency bias should not be observed for proteins or protein fragments that are not under selective immune pressure. To test this, we scored the predicted binding affinity of each CALRMUT-derived peptide or other irrelevant proteins in each individual MPN patient to generate what we have termed the Patient:Peptide Score (PPS). Briefly, the PPS of a peptide in a patient is equal to the binding score (nM) of that peptide against the MHC-I allele with the highest predicted binding affinity of the six possible MHC-I alleles for that peptide (FIG. 2A). As expected based on our initial findings, the average PPS of CALRMUT-derived peptides is elevated in CALRMUT MPN patients from both cohorts compared to control groups, where a higher score is associated with worse predicted binding (FIG. 2B,C). However, the average PPS of peptides derived from the wild-type portion of CALR is barely changed in the NEUS cohort and completely unchanged in the Danish cohort for the CALRMUT MPN patients compared to control groups (FIG. 2B,C). Similarly, the capacity to bind peptides derived from the irrelevant foreign protein neuraminidase (NA) from the influenza virus does not substantially change comparing CALRMUT, JAK^{V617F} patients and the general US Caucasian populations (FIG. 2B,C). Furthermore, when CALRMUT-derived peptides are subdivided based on predicted binding (<10000 nM) and non-binding (>10000 nM) scores, the greatest shift in average PPS occurs in predicted binding peptides (<10000 nM), suggesting selection pressure against the capacity to efficiently bind those peptides in CALRMUT patients (FIG. 2B,C). A generous cutoff of 10000 nM was used here to account for any bias in the prediction algorithm. Together, our findings suggest CALRMUT MPN patients have a skewed MHC-I allele repertoire that is less immunologically responsive to the CALRMUT protein fragment. The implication of this observation also suggests that these patients would be less likely to respond to cancer vaccines consisting of the CALRMUT fragment.

Therefore, we hypothesized that one approach to elicit an immune response against CALRMUT in these patients is to use a vaccine consisting of MHC-I binding-optimized heteroclitic peptides. To test this hypothesis, we examined the peptides with the lowest PPS in the CALRMUT MPN patients as possible candidates. We observe that the top peptide in both cohorts is the 9mer CALRMUT peptide starting at position 2 (CALR9p2) RMMRTKMRM (SEQ ID NO. 263) (FIG. 2D), which is predominantly a function of its predicted binding to frequently observed MHC-I alleles such as HLA-A*02:01 and HLA-A*03:01 (FIG. 2E,F). Therefore, based on the mean PPS of CALR9p2 in both cohorts, we hypothesized that this peptide is not able to potentiate naïve CD8+ T cells, but could be targeted by antigen-experienced CD8+ T cells.

Non-Responding Human PBMCs can Cross-React with CALR9p2 if First Primed with Heteroclitic Peptides

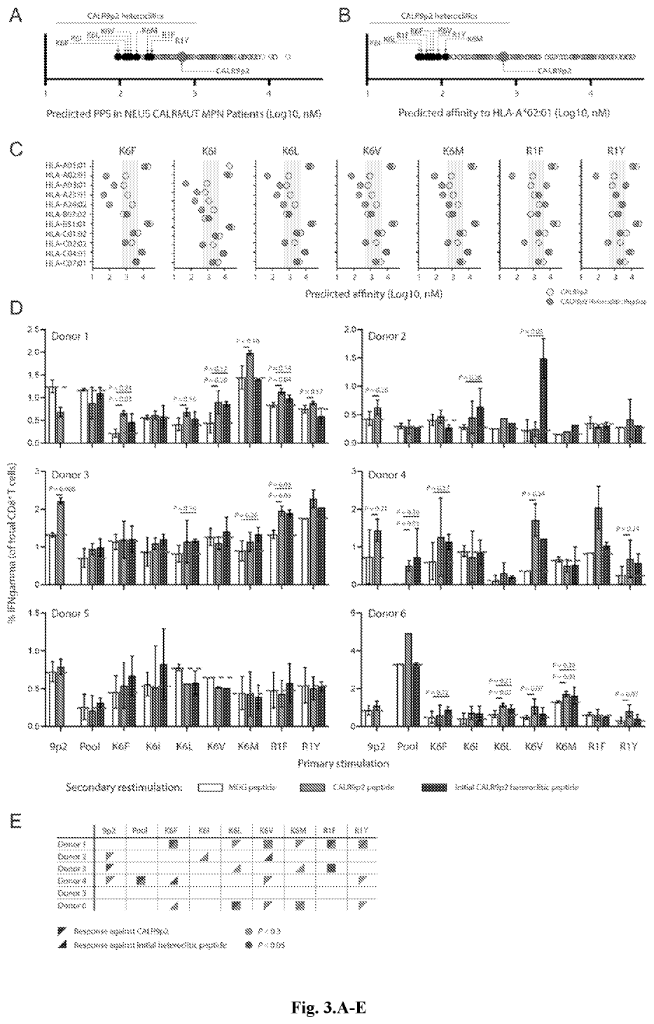

We next investigated whether heteroclitic peptides could be used to induce cross-reactivity against CALRMUT-derived peptides in human samples. To identify the best candidate CALR9p2 heteroclitic peptide, we examined the mean predicted PPS score of the NEUS CALRMUT MPN cohort to every possible CALR9p2 peptide variant containing a single amino acid substitution (FIG. 3A). Seven of the top heteroclitic CALR9p2 peptide variants were selected for testing based on their predicted binding to HLA-A*02:01 (FIG. 3B). Notably, five of those selected had a substitution at position 6 (K6) and two were selected with a substitution at position 1 (R1). A closer examination of binding predictions of each heteroclitic candidate to the top ten most frequent MHC-I alleles in CALRMUT MPN patients reveals that the K6 heteroclitic peptides could target multiple MHC-I alleles with predicted binding of the CALR9p2 ranging from 500-5000 nM, while the R1 heteroclitic peptides mostly affect HLA-A*02:01 binding affinity (FIG. 3C). We also observed that alterations of the residues at positions 1 and 6 were predicted to affect HLA-A*02:01 binding through its minor anchor sites instead of the main anchor sites at positions 2 and 9, which are predicted to already have non-ideal but still adequate residues at these positions (32, 33). Each heteroclitic CALR9p2 peptide was tested for its ability to bind to HLA-A*02:01 and confirmed to have greater binding then native CALR9p2 peptide, which had a weak binding signal compared to DMSO control (FIG. 10). However, all of the heteroclitic CALR9p2 peptides had weaker binding potential than the MART1-A2 peptide positive control.

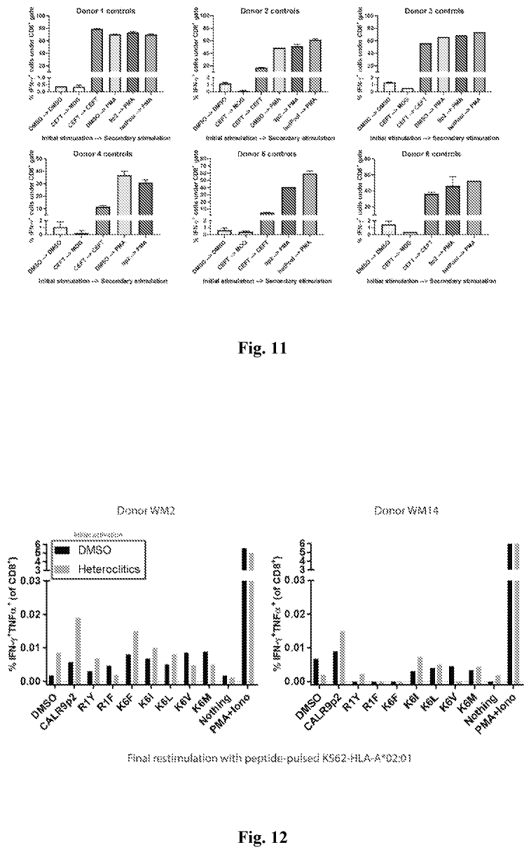

PBMCs from six healthy HLA-A*02:01 individuals with known MHC-I haplotypes and PPS were stimulated for 10 days with a cytokine cocktail in the presence of: CALR9p2, each heteroclitic peptide individually, all heteroclitic peptides pooled, or a positive control peptide mixture of T cell epitopes from Cytomegalovirus, Epstein-Barr virus, Influenza and Clostridium Tetani (CEFT). Cells were then restimulated with control peptides, initial priming peptides, or in the case of heteroclitic peptide stimulation conditions, the CALR9p2 peptide and tested for IFNγ production (FIG. 3D,E). As controls, some cells were unstimulated (DMSO), or stimulated with PMA and ionomycin (FIG. 11). Four of six samples responded to at least one heteroclitic peptide (FIG. 3D,E). Interestingly, three of the six healthy donors had significant (P<0.05) or trending (P<0.30) CD8+ T cells responses to CALR9p2 alone (Donors 2, 3 and 4), and two of them also showed significant CD8+ T cells cross-reactivity against CALR9p2 if primed with a heteroclitic peptide (Donors 3 and 4). Importantly, in two of the samples that did not respond to CALR9p2 alone (Donors 1 and 6), there was significantly detectable CD8+ T cell cross-reactivity with CALR9p2 when the PBMCs were primed with heteroclitic peptides. To test whether the heteroclitic peptides were indeed promoting cross-reactivity through HLA-A*02:01, we again activated healthy donor HLA-A*02:01-positive PBMCs using pooled heteroclitic peptides but the final restimulation was provided using peptide-pulsed K562 cells (HLA-null) transduced to only express HLA-A*02:01. In the four additional donors tested, two had a cross-reactive response against the CALR9p2 peptides, demonstrating that HLA-A*02:01 was at least one of the HLA alleles through which the heteroclitic peptides were providing a cross-reactive response (FIG. 12). Together, our results suggest that cross-reactive immunity to CALRMUT can be achieved in human cells, especially if multiple different heteroclitic peptides are utilized.

### Modeling CALRMUT MPN Patient MHC-I Allele Skewing in Mice

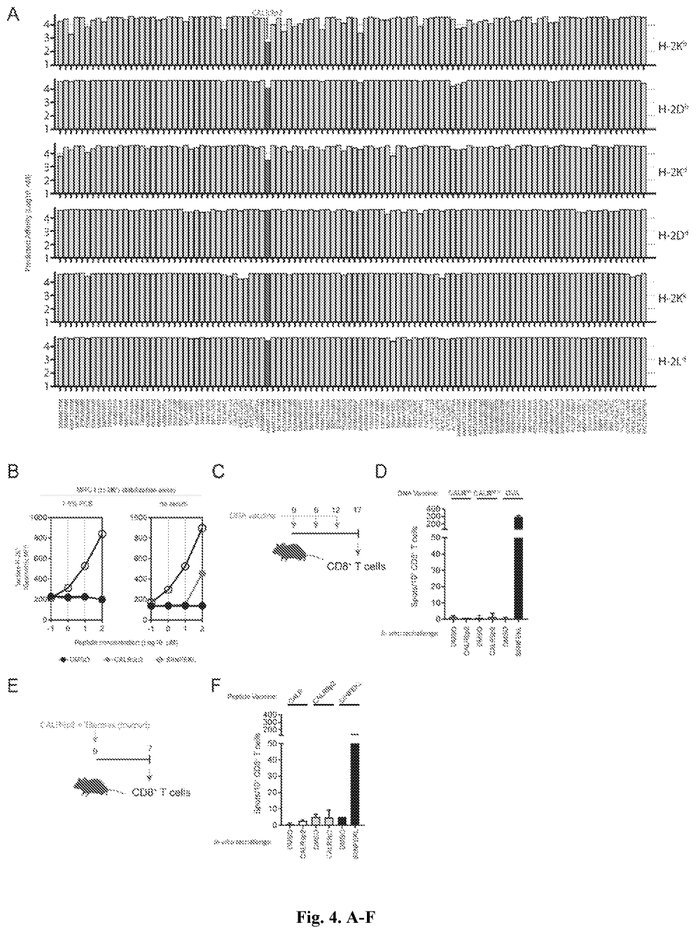

To determine whether a heteroclitic peptide cancer vaccine is a viable strategy against the CALRMUT fragment, we tested this approach in a pre-clinical mouse model mimicking CALRMUT MPN MHC-I allele skewing. We analyzed the predicted binding of all CALRMUT-derived peptides against all murine MHC-I alleles for which predictions are possible. We found no strong binding peptide (<500 nM) to all murine MHC-I alleles but did observe that H-2Kb has a weakly binding predicted affinity to CALR9p2. When tested for its ability to stabilize MHC-I in the H-2Kb-expressing TAP-deficient RMA/S cell line, CALR9p2 did not elicit detectable H-2Kb stabilization compared to the control strong binding chicken ovalbumin (OVA)-derived peptide SIINFEKL (SEQ ID NO. 287) (FIG. 4B). However, when serum was omitted from the assay, H-2Kb was stabilized only when the highest concentration of peptide (100 μg/mL), suggesting poor but still detectable binding (FIG. 4B). To investigate whether the CALRMUT protein fragment is immunogenic in vivo, we immunized H2-Kb-expressing C57BL/6J (B6) mice with a DNA vaccine (FIG. 4C) encoding the full-length 52 base pair deletion variant of the CALRMUT sequence (4-6) and analyzed CALR9p2 peptide-specific CD8+ T cells from draining lymph nodes. Compared to the immunogenic CD8+ T cell response against the SIINFEKL (SEQ ID NO. 287) peptide in OVA-immunized mice, mice immunized with the full-length CALRMUT sequence did not elicit any CD8+ T cell response against CALR9p2 (FIG. 4D). Likewise, mice immunized in the footpad (FIG. 4E) with the CALR9p2 peptide emulsified in the Titermax® adjuvant also did not elicit any CALR9p2-specific CD8+ T cell response compared to SIINFEKL(SEQ ID NO. 287)-immunized mice (FIG. 4F).

We posited that this mouse model is a good preclinical model candidate of CALRMUT MPN patients mimicking an MHC-I skewed haplotype because we observe poor but detectable binding of CALR9p2 to H-2Kb but no vaccine-induced CALR9p2-specific CD8+ T cell responses in B6 mice.

Full-Length CALRMUT Variant does not have Dominant-Negative Activity

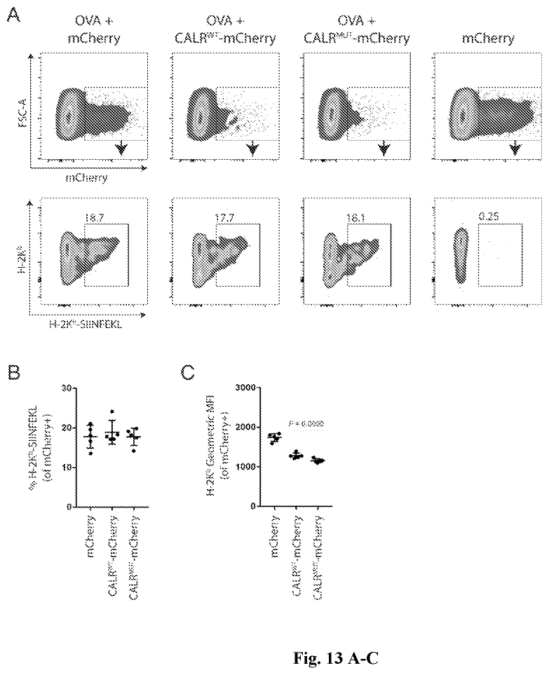

We wanted to investigate whether the CALRMUT fragment itself could inhibit antigen presentation. Wildtype CALR is required for antigen-presentation in healthy cells (34, 35) and the full-length CALRMUT is reported to be non-functional with respect to peptide loading (35). However, nearly all CALRMUT-positive MPN tumors are heterozygous (4, 5) and therefore have one wild-type copy of CALR, yet it is unknown whether CALRMUT acts as a dominant-negative with respect to its role in antigen presentation. To exclude this possibility, we co-transfected the murine B16F10 cells with the DNA sequences encoding OVA and either CALRWT-mCherry, CALRMUT-mCherry or the mCherry constructs, and measured surface expression of H-2Kb-presented SIINFEKL peptide. We observed that cells transfected with the CALRMUT variant had an equal percentage of H-2Kb-SIINFEKL (SEQ ID NO. 287) peptides expressing cells (FIG. 13B), suggesting that the CALRMUT variant does not inhibit antigen presentation. Interestingly, we observe a marginal decrease in total surface MHC-I expression (FIG. 12C), suggesting some effect of the CALRMUT variant, although it is unclear whether such a small reduction in MHC-I would have any functional effect. As a result, we conclude that CALRMUT does not prevent tumor cells from presenting antigens and suggest that CALRMUT tumors could respond to cancer vaccines, if the correct antigen is selected and is expressed by the tumor.

### C57BL/6J-Optimized Heteroclitic CALR9p2 Peptide Elicits Cross-Reactive Immunity in Mice

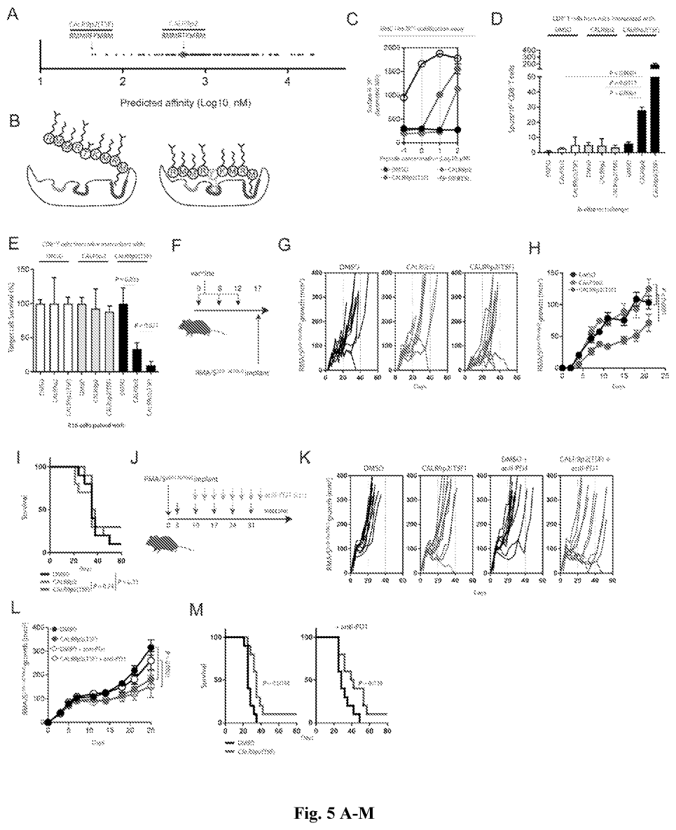

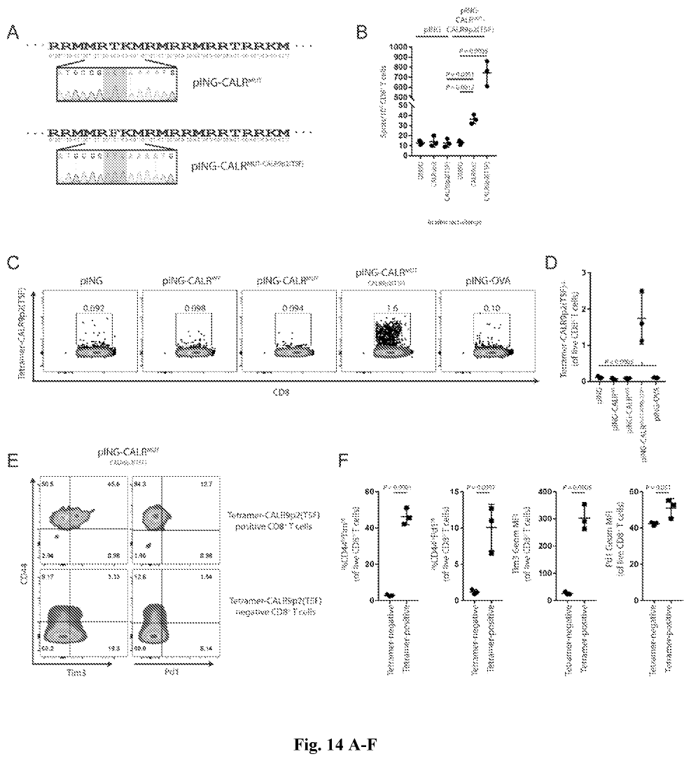

To identify the best candidate CALR9p2 heteroclitic peptide in C57BL/6J (B6) mice, we examined the predicted binding affinity of H-2Kb to every possible CALR9p2 peptide variant containing a single amino acid substitution. We observed that the variant with the strongest predicted affinity has a threonine (T) to phenylalanine (F) substitution at position 5 (T5F) of the CALR9p2 peptide (FIG. 5A). The CALR9p2-T5F peptide has the amino acid sequence of SEQ ID NO. 40. This is consistent with previous studies showing that this site is a major anchor residue for H-2Kb (FIG. 5B) (19, 36, 37). When investigated for its ability to stabilize H-2Kb in RMA/S cells, CALR9p2(T5F) demonstrated approximately a tenfold greater H-2Kb stabilization compared to CALR9p2 (FIG. 5C). We then tested whether CALR9p2(T5F) could elicit a cross-reactive CALR9p2 immune response. Mice immunized with a single dose of CALR9p2(T5F) elicited a CD8+ T cell capable of cross-reacting with CALR9p2 in vitro (FIG. 5D) and killing tumor cells pulsed with the CALR9p2 peptide (FIG. 5E). Likewise, mice immunized by DNA vaccine against CALRMUT encoding the CALR9p2(T5F) variant also elicited a cross-reactive response against CALR9p2 (FIG. 14A). CALR9p2(T5F)-specific CD8+ T cells by tetramer staining (FIG. 14D) had higher levels of the activation markers CD44, Tim3 and Pd1. Importantly, the ability to mount an antigen specific response against the full-length antigen demonstrated that the full-length CALRMUT sequence can be endogenously processed and presented, which had not previously been proven directly.

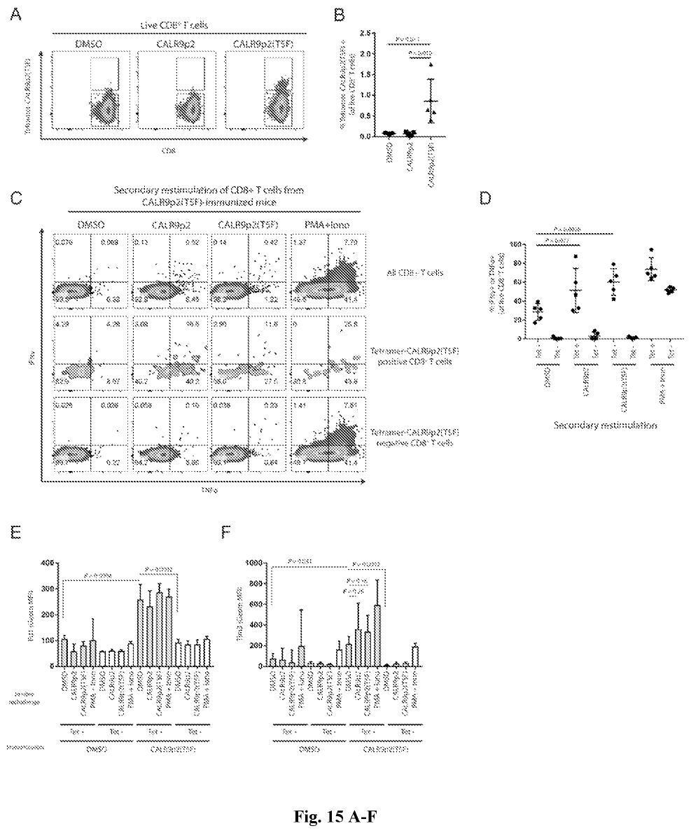

To confirm that the same TCR clones were recognizing both CALR9p2(T5F) and CALR9p2, CD8+ T cells from CALR9p2(T5F)-immunized mice were restimulated in vivo with CALR9p2, and CALR9p2(T5F)-tetramer-specific CD8+ T cells were examined for IFNγ restimulation. The only CALR9p2-potentiated CD8+ T cells were those also staining for the CALR9p2(T5F)-tetramer (FIG. 15A-D). Notably, both the CALR9p2-specific and CALR9p2(T5F)-specific CD8+ T cells had equal levels of Tim3 and Pd1 after restimulation.

To test the ability of a CALRMUT heteroclitic peptide vaccine to elicit a cross-reactive anti-tumor response in vivo, we used the newly developed PresentER antigen minigene system (38). Here, the nucleotide sequence of the CALR9p2 peptide is cloned downstream of an ER signal sequence (SS) and virally transduced into TAP-deficient RMA/S cells (RMA/SpER-CALR9p2). Once expressed, the peptide-SS is shuttled into the ER and the peptide is cleaved from the SS, releasing CALR9p2 into the ER where it can be loaded into MHC-I, assuming binding is possible. When mice were given three doses of the heteroclitic peptide vaccine prior to tumor implantation, RMA/SpER-CALR9p2 tumors grew significantly slower than those injected into mice given adjuvant alone or with the CALR9p2 peptide, which both grew at the same rate (FIG. 5F,G) although differences in survival were not significant (FIG. 5H). However, when RMA/SpER-CALR9p2 tumors were allowed to grow before mice received multiple therapeutic doses of the heteroclitic peptide vaccine (FIG. 5I), tumors grew significantly slower (FIG. 5J,K) and mice had improved survival (FIG. 5L) compared to the adjuvant alone condition. Importantly, the effect of the vaccine was even more prominent when the immunization is administered with immune checkpoint blockade using a PD-1 antibody (FIG. 5I-K).

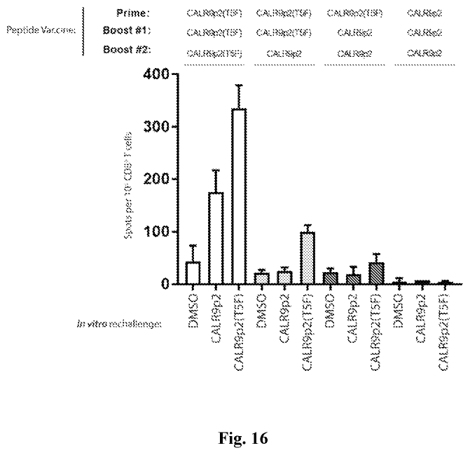

Interestingly, the therapeutic vaccine had greater efficacy than the prophylactic vaccine. As this was unexpected, we hypothesized that CALR9p2-specific cross-reactive CD8+ T cells were diminishing in efficacy over time and that the available CALR9p2 antigen present in the tumor cells was not generating a strong memory response. To investigate this further, we immunized mice with three doses of the heteroclitic CALR9p2(T5F) peptide vaccine and compared cross-reactive potential in conditions where mice instead received CALR9p2 peptide boosts following an initial CALR9p2(T5F) priming dose. Consistent with the prophylactic vaccine results, mice that received an initial CALR9p2(T5F) followed by two CALR9p2 boosts had no detectable cross-reactive CD8+ T cell responses to CALR9p2 in vitro and a very small response to the CALR9p2(T5F) peptide (FIG. 16). On the other hand, mice that received two doses of CALR9p2(T5F) and a final CALR9p2 boost had no detectable cross-reactive CD8+ T cells but an intermediate response to CALR9p2(T5F). Therefore, it appears that cross-reactive immunity to the CALR9p2 peptide in the context of B6 mice is a function of time-from-last CALR9p2(T5F) dose.

Together, the results of this study provide proof of principle for the use of a heteroclitic peptide cancer vaccine strategy for tumor cells expressing CALRMUT antigens. Such a vaccine could provide a valuable non-redundant benefit in CALRMUT MPN patients, as there are currently no rationally designed treatments specially targeting the CALR mutation. While the JAK1/JAK2 inhibitor, ruxolitinib is approved by the FDA for the treatment of patients with MPN, this approval was granted primarily based on symptomatic benefits (53, 54). Although CALRMUT MPN patients demonstrate clinical responses to ruxolitinib, there is no reduction in CALRMUT allele burden following JAK2 inhibition and as a result ruxolitinib does not have substantial disease-modifying activity in MPN and is not curative (55). Mutations in CALR are disease-initiating in MPN and often occur as the sole mutation (4).

We plan to perform clinical trials. Initial clinical trials of heterolytic CALRMUT directed vaccination approaches as described herein will focus primarily on safety and will be performed in patients with more advanced MPN. A longer-term goal will be to treat CALRMUT MPN patients early in the course of their disease before genetic and clonal evolution has occurred. By directing autologous immune responses specifically against CALRMUT, peptide vaccination offers the potential to preferentially target the disease-initiating MPN stem cell in patients, which is a deficiency of current MPN drugs, including JAK inhibitors. Accordingly, CALRMUT targeted peptide vaccination offers the potential to definitely eradicate MPN to cure the disease.

Importantly, while our work suggests that certain patients are not likely to respond to a cancer vaccine composed solely of the CALRMUT fragment due to MHC-I skewing, we believe that these patients are likely to respond to a heteroclitic peptide cancer vaccine as demonstrated in our pre-clinical model. Furthermore, as our data shows that this strategy can be enhanced with immune checkpoint blockade (anti-PD-1/PD-L1), an off-the-shelf vaccine composed of one or more of the heteroclitic CALRMUT peptides described herein in combination with anti-PD-1 or other checkpoint inhibitor therapies, can be a viable strategy for CALRMUT MPN patients.

### Methods

### Patient Samples

Approval was obtained for the use of patient-derived specimens and access to clinical data extracted from patient charts by the Institutional Review Boards at Memorial Sloan Kettering Cancer Center, the Dana-Farber Cancer Institute and the Massachusetts General Hospital, as well as by the Danish Regional Science Ethics Committee. All patients analyzed in this study were diagnosed with MPN and tested positive for the CALRMUT or JAK^{V617F} mutations.

### Mice

C57BL/6J mice were purchased from The Jackson Laboratory (Sacramento, Calif.). Mouse experiments were performed in accordance with institutional guidelines under a protocol approved by the Memorial Sloan-Kettering Cancer Center Institutional Animal Care and Use Committee. All mice were maintained in a pathogen-free facility according to the National Institutes of Health Animal Care guidelines.

### MHC Allele Frequencies

For the NEUS cohort, MHC genotypes were manually extracted from patient charts. For the Danish cohort, HLA class I genotypes were determined by next-generation sequencing performed on bio-banked samples collected for a previous study (56) using recently described methods (57). In cases where NMDP allele codes were used instead of the World Health Organization nomenclature, the conversion was done according to https://bioinformatics.bethematchclinical.org/hla-resources/allele-codes/allele-code-lists/. If multiple alleles were plausible for a given NMDP allele code, we selected the most likely allele based on ethnicity (typically around 99% confidence, based on known frequencies in the general population (17)). MHC allele frequency for each HLA gene (A, B, C, DQ, and DR) was broadly calculated as the number of each specific allele divided by the number of the total allele in that cohort (2n per individual). In rare samples, certain patients had incomplete haplotype information where one or more alleles were unknown or incomplete. If the allele was missing, it was censored from the number of total alleles. If locus and group were known (ex: HLA-A*02) but the exact protein was unknown (ex: HLA-A*02:XX), this allele was censored from the frequency calculation only for alleles from the same group. For an MHC allele to be considered positively skewed, it was required to have an allele frequency of >0.05 in CALRMUT MPN cases and have >0.2 fold frequency increase compared to both the JAK2^{V617F} and US Caucasian population groups allele frequency in the NEUS cohort, or a >0.125 fold frequency increase compared to the JAK2^{V617F} group allele frequency in the Danish cohort. Likewise, for an MHC allele to be negatively skewed, it was required to have a frequency of at least 0.05 in both the JAK2^{V617F} and US Caucasian population groups for the NEUS cohort, or just the JAK2^{V617F} group for the Danish cohort, and have >0.2 fold decrease in the CALRMUT MPN compared to both the JAK2^{V617F} and US Caucasian population groups allele frequency in the NEUS cohort, or a >0.125 fold frequency decrease compared to the JAK2^{V617F} group allele frequency in the Danish cohort. Principal component analysis was calculated in R and plotted in Graphpad Prism 7. All data processing and analysis were performed using the R version 3.3.2 Sincere Pumpkin Patch and GraphPad Prism v7.

### Binding Affinity Predictions and Patient:Peptide Score (PPS)

pMHC-I binding predictions were collected using NetMHCpan v3 (32) for human MHC-I alleles and NetMHC v4 (36) for murine MHC-I alleles. pMHC-II binding predictions were collected using NetMHCIIpan v3.2 (58). To calculate the PPS, peptide affinities for all six possible MHC-I alleles were identified and only the lowest affinity value was retained. The protein fragment RRMMRTKMRMRRMRRTRRKMRRKMSPARPRTSCREACLQGWTEA (SEQ ID NO. 288) was used for the CALRMUT. For the CALRWT sequence, amino acids 1-361 of the UniProt sequence P27797 was used. For the Influenza neuraminidase sequence, the full UniProt sequence D7ED91 was used. All subsequent data processing and analyses were performed using the R version 3.3.2 Sincere Pumpkin Patch and GraphPad Prism v7.

### MHC-I Stabilization Assay

To test peptide binding to murine H-2Kb or human HLA-A*02:01, TAP-deficient RMA/S and T2 cells were used, respectively, as previously reported (59, 60). Briefly, RMA/S cells (61) were serum-starved in serum-free RPMI media overnight at 31° C., and peptides were added at indicated final concentrations, followed by 30 minutes at 31° C. and another 3 hours at 37° C. before measuring H-2Kb by flow cytometry (BD; Clone AF6-88.5). T2 cells (62) were serum-starved in serum-free RPMI media overnight and cells were added at indicated final concentrations for 16 hours before measuring HLA-A*02:01 levels by flow cytometry (BD; Clone BB7.2). The SIINFEKL (SEQ ID NO. 287) peptide was acquired as a custom order from Genscript. The MART1-A2 peptide (ELAGIGILTV, SEQ ID NO. 289) was purchased from JPT Peptide Technologies.

### Murine Immunizations and IFNγ ELISpot Assay

All peptides were purchased as custom peptide synthesis orders from GenScript at a purity of >98% and resuspend at 10 mg/mL in DMSO (Sigma). For peptide vaccines, peptides were diluted in PBS and emulsified with Titermax® (Titermax USA, Inc) at a 1:1 ratio immediately prior to immunization, such that each dose was composed of 10 μg peptide in a total volume of 25 uL. In control (DMSO) immunization, an equivalent volume of DMSO is substituted for the diluted peptide in the TiterMax® emulsion. For subsequent in vitro assays, draining inguinal and popliteal lymph nodes were collected at indicated time points and CD8+ T cells were isolated using positive magnetic sorting using mouse CD8a (Ly-2) MicroBeads (Miltenyi Biotec). DNA vaccines were performed using a gene gun as previously described (27, 63) according to indicated time points. Briefly, mice received four injections (400 lbs/inch2) of DNA-coated gold particles into the abdominal region of the skin for a total 4 μg of DNA per dose. DNA plasmids encoding the wildtype or 52 base-pair deletion CALRMUT sequences fused with the flag sequence are as previously described (64). For pING-OVA, the full-length chicken ovalbumin sequence was cloned into the pING plasmid (65). CD8+ T cells were collected as before, but only from draining inguinal lymph nodes. To test for antigen-specificity, the mouse IFNγ ELISpot set (BD) was used according to the manufacturer's instructions. Briefly, CD8+ T cells were frozen immediately after purification in FBS containing 10% DMSO, thawed one day prior to restimulation and allowed to recover overnight in 20 U/mL IL-2 (Peprotech) RPMI-1640 medium containing 10% fetal bovine serum (FBS), Na-Pyruvate, L-glutamine and Penicillin/Streptomycin. As a source of antigen-presenting cells (APCs), splenocytes from naïve mice were depleted of T cells using magnetic microbeads for CD8a (Ly2) and CD4 (L3T4) (Miltenyi Biotec), pulsed for one hour with 100 μg/mL peptide at 37° C. followed by a wash. For each well, 105 CD8+ T cells were co-culture with 1×105-3×105 peptide-pulsed APCs and incubated for approximately 18 hours. Spots were counted using the ImmunoSpot analyzer (Cellular Technology Limited).

### SIINFEKL (SEQ ID NO. 287) H-2Kb Expression

B16F10 were co-transfected with equal parts pING-OVA and pCMV-Sport6-CALR constructs fused to mCherry, which are previously described (64), using the Megatran 1.0 transfection reagent (Origene). Each construct was mixed with the transfection reagent separately such that all cells received the same amount of pING-OVA construct. B16F10 cells were originally obtained from I. Fidler (M. D. Anderson Cancer Center) and cultured in RPMI 1640 medium supplemented with 7.5% inactivated FBS, 1×non-essential amino acids and 2 mM L-glutamine. One day after transfection, cells were stained by flow cytometry with H-2Kb (BD; Clone AF6-88.5) and H-2Kb-SIINFEKL (SEQ ID NO. 287) (Biolegend; Clone 25-D1.16).

### Tumor Growth Experiments

RMA/S cells were maintained in RPMI 1640 medium supplemented with 7.5% inactivated FBS, 1×non-essential amino acids and 2 mM L-glutamine. The DNA sequence encoding the CALR9p2 peptide (bold) was cloned into the PresentER-IRES-GFP (38) construct using the following oligo:

(SEQ ID NO. 290)
5′GGCCGTATTGGCCCCGCCACCTGTGAGCGG
GAGGATGATGAGGACAAAGATGAGGATGTAAGGCC
AAACAGGCC-3′

following SfiI digestion and T4 ligation (New England Biolabs). The resulting construct was used to generate retrovirus by co-transfection with pCL-Ampho into ecotropic Pheonix cells (ATCC). Viral supernatants were collected at 48 and 72 hours, pooled and Retro-X Concentrator (Takara Bio USA)-concentrated retrovirus was used to transduce RMA/S cells by spinoculation using polybrene (Sigma). GFP-positive cells were FACS sorted (BD FACSAria III) and cultured in 4 μg/mL puromycin (Gibco) media. A total of 5×106 cells were injected subcutaneously in the flank of mice. For anti-PD1 treatment, 250 μg of RMP1-14 was injected intra-peritoneally in PBS at indicated time points.

### Human PBMC In Vitro Restimulation

Freshly isolated or thawed cryopreserved healthy donor PBMCs were restimulated with cytokines and peptides as previously described (14). Briefly, on day 0, PBMCs were resuspended in X-VIVO15 media (Lonza) and seeded at 105 per well of a 96 U-bottom plate with 1000 IU/mL GM-CSF (Sanofi), 500 IU/mL IL-4 (R&D Systems) and 50 ng/mL Flt3L (R&D Systems). On day 1, media was refreshed with 0.1 μg/mL LPS (Invivogen), 10 μM R848 (Invivogen), 5 μg/mL IL-10 (R&D Systems) and 1 μg/mL of indicated peptides, and incubated for 24 hours. The CMV, EBV, Flu, Tetanus (CEFT) and the myelin oligodendrocyte glycoprotein (MOG) peptide pools (JPT Technologies) were used as positive and negative controls, respectively. On days 2 and 5, half the media was refreshed with RPMI (Gibco) containing 10% human serum (Gemini Bio-Products), 10 ug/ml gentamycin (Gibco), HEPES (Gibco), GlutaMAX (Gibco) and hIL-2 and hIL-7 to a final concentration of 10 IU/mL and 10 ng/mL, respectively (R&D Systems). On day 8, the media was refreshed without cytokines. On day 10, PBMCs were restimulated with corresponding peptides in the presence of 1 μg/mL of anti-hCD28 and anti-hCD49d (BD Biosciences). As controls, some cells were stimulated with PMA (Sigma-Aldrich, 50 ng/mL) and ionomycin (Sigma-Aldrich, 1 μg/mL). For intracellular staining, monensin and brefeldin A (BD Biosciences) were added 1 hour after restimulation cells and culture left to incubate for another 12 hours. Cells were then stained for CD3 (Clone: OKT3, FITC), CD4 (Clone: RPA-T8, APC) and CD8a (Clone: RPA-T4, BV785), permeabilized and fixed with BD Cytofix/Cytoperm™ reagents according to manufacturer's protocol and subsequently stained for IFNγ (Clone: B27, PE) and TNFα (Clone: Mab11, PE/Cy7). All antibodies were purchased BioLegend. LIVE/DEAD™ Fixable Blue Dead Cell Stain Kit by Thermo Fischer Scientific was used for live and dead cell discrimination. Data was acquired using the BD Fortessa and the data was analyzed on FlowJo V10 (TreeStar). For the ELISpot, methods are as previously described (14) using the follower 15-mer peptides that cover the entire CALRMUT fragment: RTRRMMRTKMRMRRM (SEQ ID NO. 291), MMRTKMRMRRMRRTR (SEQ ID NO. 292), TKMRMRRMRRTRRK (SEQ ID NO. 293), RMRRMRRTRRKMRRK (SEQ ID NO. 294), MRRTRRKMRRKMSPA (SEQ ID NO. 295), RRKMRRKMSPARPRT (SEQ ID NO. 296), RRKMSPARPRTSCRE (SEQ ID NO. 297), SPARPRTSCREACLQ (SEQ ID NO. 298), PRTSCREACLQGWTE (SEQ ID NO. 299), TSCREACLQGWTEA (SEQ ID NO. 300).

### Statistical Analysis

Details of the study outline, sample size, and statistical analysis are shown in the main Example text, above and in, the Figures and Brief Description of the Figures. To calculate significance in distribution of MHC frequencies, Barnard's unconditional test and the chi-square test were used as indicated in R using the barnard.test function (two-tail) from the Barnard package and as well as the base chisq.test function. The R version 3.3.2 Sincere Pumpkin Patch was used. For unpaired Student's t tests, area under the curve calculations and log-rank survival test, GraphPad Prism v7 was used.

### Example 2

Additional CALR^{MUT} heteroclitic peptides were designed utilizing a novel algorithm that we designed specifically to identify and select heteroclitic peptides likely to be useful for vaccination of as large a proportion of the general population as possible. In brief, this entailed first identifying native CALR^{MUT} peptides likely to be good starting points for the generation of heteroclitic mutants/derivatives based on their predicted utility for vaccination of the greatest number of patients (based on HLA-I allele diversity). Then mutations of these “native” peptides were evaluated based on certain criteria to identify heteroclitic mutants. The conditions for a mutant peptide to be considered heteroclitic in a given individual were: 1) that the native peptide from which it was derived had a predicted binding affinity to a given HLA-I expressed by that individual of between 500 nM and 2000 nM (i.e. intermediate binding HLA-I), and 2) that the mutation altered the predicted binding affinity of the peptide to at least one of the intermediate binding HLA-Is in that individual to <500 nM. From that first subset of mutants identified as being heteroclitic using those two criteria, a second subset having a predicted HLA-I binding affinity of <100 nM was identified—as these were predicted to have the best immunogenicity. Our algorithm assigned scores to all of the heteroclitic peptides based on various criteria including their likely utility across multiple ethnic groups (Caucasian, African, Asian and Hispanic) and across multiple HLA phenotypes. The amino acid sequences of the subset of 262 heteroclitic derivatives having the best scores as determined by this method are provided in Table 1 (SEQ ID NOs 1-262). An analysis of the amino acid sequences of these 262 peptides in comparison to the native CALR^{MUT} peptide from which they were derived, identified certain common features of the mutant heteroclitic derivatives—which common features are described by the various consensus amino acid sequences provided in Table 2. Seven of the mutant heteroclitic derivatives were tested functionally in living human cells and/or in mice, as described in Example 1, providing proof of concept for the design approach and for the utility of the designed mutant heteroclitic peptides.

### REFERENCE LIST

1. R. L. Levine, D. G. Gilliland, Myeloproliferative disorders. Blood 112, 2190-2198 (2008).
2. P. J. Campbell, A. R. Green, The myeloproliferative disorders. N Engl J Med 355, 2452-2466 (2006).
3. Rampal et al., Integrated genomic analysis illustrates the central role of JAK-STAT pathway activation in myeloproliferative neoplasm pathogenesis. Blood 123, e123-133 (2014).
4. Nangalia et al., Somatic CALR mutations in myeloproliferative neoplasms with nonmutated JAK2. N Engl J Med 369, 2391-2405 (2013).
5. Klampfl et al., Somatic mutations of calreticulin in myeloproliferative neoplasms. N Engl J Med 369, 2379-2390 (2013).
6. Elf et al., Mutant Calreticulin Requires Both Its Mutant C-terminus and the Thrombopoietin Receptor for Oncogenic Transformation. Cancer Discov 6, 368-381 (2016).
7. Chachoua et al., S. N. Constantinescu, Thrombopoietin receptor activation by myeloproliferative neoplasm associated calreticulin mutants. Blood 127, 1325-1335 (2016).
8. Araki et al., Activation of the thrombopoietin receptor by mutant calreticulin in CALR-mutant myeloproliferative neoplasms. Blood 127, 1307-1316 (2016).
9. How & Hobbs, A. Mullally, Mutant calreticulin in myeloproliferative neoplasms. Blood 134, 2242-2248 (2019).
10. Holmstrom et al., The CALR exon 9 mutations are shared neoantigens in patients with CALR mutant chronic myeloproliferative neoplasms. Leukemia 30, 2413-2416 (2016).
11. Holmstrom et al., The calreticulin (CALR) exon 9 mutations are promising targets for cancer immune therapy. Leukemia 32, 429-437 (2018).
12. Tubb, et al., Isolation of T cell receptors targeting recurrent neoantigens in hematological malignancies. Journal for immunotherapy of cancer 6, 70 (2018).
13. Holmstrom et al., High frequencies of circulating memory T cells specific for calreticulin exon 9 mutations in healthy individuals. Blood Cancer J 9, 8 (2019).
14. Cimen Bozkus, et al., Immune Checkpoint Blockade Enhances Shared Neoantigen-Induced T Cell Immunity Directed against Mutated Calreticulin in Myeloproliferative Neoplasms. Cancer Discov, (2019).
15. Schischlik et al., Mutational Landscape of the Transcriptome Offers Putative Targets for Immunotherapy of Myeloproliferative Neoplasms. Blood, (2019).
16. Madden, The three-dimensional structure of peptide-MHC complexes. Annu Rev Immunol 13, 587-622 (1995).
17. Gragert et al., Six-locus high resolution HLA haplotype frequencies derived from mixed-resolution DNA typing for the entire US donor registry. Human immunology 74, 1313-1320 (2013).
18. Falk et al., Allele-specific motifs revealed by sequencing of self-peptides eluted from MHC molecules. Nature 351, 290-296 (1991).
19. Rammensee et al., MHC ligands and peptide motifs: first listing. Immunogenetics 41, 178-228 (1995).
20. Boulanger et al., Dalchau, A Mechanistic Model for Predicting Cell Surface Presentation of Competing Peptides by MHC Class I Molecules. Front Immunol 9, 1538 (2018).
21. Ericsson et al., Differential activation of phospholipase C-gamma 1 and mitogen-activated protein kinase in naive and antigen-primed CD4 T cells by the peptide/MHC ligand. Journal of immunology 156, 2045-2053 (1996).
22. Kimachi et al., The minimal number of antigen-major histocompatibility complex class II complexes required for activation of naive and primed T cells. Eur J Immunol 27, 3310-3317 (1997).
23. London et al., Functional responses and costimulator dependence of memory CD4+ T cells. Journal of immunology 164, 265-272 (2000).
24. Pihlgren et al., Resting memory CD8+ T cells are hyperreactive to antigenic challenge in vitro. The Journal of experimental medicine 184, 2141-2151 (1996).
25. Rogers et al., Qualitative changes accompany memory T cell generation: faster, more effective responses at lower doses of antigen. Journal of immunology 164, 2338-2346 (2000).
26. Slifka et al., Functional avidity maturation of CD8(+) T cells without selection of higher affinity TCR. Nat Immunol 2, 711-717 (2001).
27. Dyall et al., Heteroclitic immunization induces tumor immunity. The Journal of experimental medicine 188, 1553-1561 (1998).
28. Rosenberg, et al., White, Immunologic and therapeutic evaluation of a synthetic peptide vaccine for the treatment of patients with metastatic melanoma. Nature medicine 4, 321-327 (1998).
29. England, et al., Molecular analysis of a heteroclitic T cell response to the immunodominant epitope of sperm whale myoglobin. Implications for peptide partial agonists. Journal of immunology 155, 4295-4306 (1995).
30. Boehncke, et al., The importance of dominant negative effects of amino acid side chain substitution in peptide-MHC molecule interactions and T cell recognition. Journal of immunology 150, 331-341 (1993).
31. Gonzalez-Galarza, et al., Allele frequency net 2015 update: new features for HLA epitopes, KIR and disease and HLA adverse drug reaction associations. Nucleic Acids Res 43, D784-788 (2015).
32. Nielsen & Andreatta, NetMHCpan-3.0; improved prediction of binding to MHC class I molecules integrating information from multiple receptor and peptide length datasets. Genome Med 8, 33 (2016).
33. Hoof, et al., NetMHCpan, a method for MHC class I binding prediction beyond humans. Immunogenetics 61, 1-13 (2009).
34. Gao, et al., Assembly and antigen-presenting function of MHC class I molecules in cells lacking the ER chaperone calreticulin. Immunity 16, 99-109 (2002).
35. Arshad, et al., Tumor-associated calreticulin variants functionally compromise the peptide loading complex and impair its recruitment of MHC-I. J Biol Chem 293, 9555-9569 (2018).
36. Andreatta & Nielsen, Gapped sequence alignment using artificial neural networks: application to the MHC class I system. Bioinformatics 32, 511-517 (2016).
37. Nielsen, et al., Reliable prediction of T-cell epitopes using neural networks with novel sequence representations. Protein Sci 12, 1007-1017 (2003).
38. Gejman, et al., Rejection of immunogenic tumor clones is limited by clonal fraction. Elife 7, (2018).
39. Dunn, et al., The three Es of cancer immunoediting. Annu Rev Immunol 22, 329-360 (2004).
40. Teng, et al., From mice to humans: developments in cancer immunoediting. The Journal of clinical investigation 125, 3338-3346 (2015).
41. Hanahan, et al., Hallmarks of cancer: the next generation. Cell 144, 646-674 (2011).
42. O'Donnell, et al., Cancer immunoediting and resistance to T cell-based immunotherapy. Nature reviews. Clinical oncology 16, 151-167 (2019).
43. Efremova, et al., Targeting immune checkpoints potentiates immunoediting and changes the dynamics of tumor evolution. Nat Commun 9, 32 (2018).
44. Rizvi, et al., Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol 36, 633-641 (2018).
45. Turajlic et al., Insertion-and-deletion-derived tumour-specific neoantigens and the immunogenic phenotype: a pan-cancer analysis. Lancet Oncol 18, 1009-1021 (2017).
46. Linnebacher, et al., Frameshift peptide-derived T-cell epitopes: a source of novel tumor-specific antigens. International journal of cancer. Journal international du cancer 93, 6-11 (2001).
47. Posthuma, et al., Niederwieser, HLA-B8 and HLA-A3 coexpressed with HLA-B8 are associated with a reduced risk of the development of chronic myeloid leukemia. The Chronic Leukemia Working Party of the EBMT. Blood 93, 3863-3865 (1999).
48. Kuzelova, et al., Altered HLA Class I Profile Associated with Type A/D Nucleophosmin Mutation Points to Possible Anti-Nucleophosmin Immune Response in Acute Myeloid Leukemia. PloS one 10, e0127637 (2015).
49. Sharma, et al., Primary, Adaptive, and Acquired Resistance to Cancer Immunotherapy. Cell 168, 707-723 (2017).
50. Rodig, et al., MHC proteins confer differential sensitivity to CTLA-4 and PD-1 blockade in untreated metastatic melanoma. Sci Transl Med 10, (2018).
51. Li, et al., T cell receptor signalling in the control of regulatory T cell differentiation and function. Nature reviews. Immunology 16, 220-233 (2016).
52. Klebanoff, et al., Therapeutic cancer vaccines: are we there yet? Immunological reviews 239, 27-44 (2011).
53. Harrison, et al., JAK inhibition with ruxolitinib versus best available therapy for myelofibrosis. N Engl J Med 366, 787-798 (2012).
54. Verstovsek, et al., A double-blind, placebo-controlled trial of ruxolitinib for myelofibrosis. N Engl J Med 366, 799-807 (2012).
55. Guglielmelli, et al., Investigators, Ruxolitinib is an effective treatment for CALR-positive patients with myelofibrosis. British journal of haematology 173, 938-940 (2016).
56. Knudsen et al, Long-Term Efficacy and Safety of Recombinant Interferon Alpha-2 Vs. Hydroxyurea in Polycythemia Vera: Preliminary Results from the Three-Year Analysis of the Daliah Trial—a Randomized Controlled Phase III Clinical Trial. Blood 132, 580-580 (2018).
57. Jan, et al., Recurrent genetic HLA loss in AML relapsed after matched unrelated allogeneic hematopoietic cell transplantation. Blood Adv 3, 2199-2204 (2019).
58. Jensen, et al., Improved methods for predicting peptide binding affinity to MHC class II molecules. Immunology 154, 394-406 (2018).
59. Houghton, et al., Immunological validation of the EpitOptimizer program for streamlined design of heteroclitic epitopes. Vaccine 25, 5330-5342 (2007).
60. Guevara-Patino, et al., A. N. Houghton, Optimization of a self antigen for presentation of multiple epitopes in cancer immunity. The Journal of clinical investigation 116, 1382-1390 (2006).
61. Hosken & Bevan, An endogenous antigenic peptide bypasses the class I antigen presentation defect in RMA-S. The Journal of experimental medicine 175, 719-729 (1992).
62. Salter, et al., Cresswell, Genes regulating HLA class I antigen expression in T-B lymphoblast hybrids. Immunogenetics 21, 235-246 (1985).
63. Weber, et al., Tumor immunity and autoimmunity induced by immunization with homologous DNA. The Journal of clinical investigation 102, 1258-1264 (1998).
64. Elf, et al., Defining the requirements for the pathogenic interaction between mutant calreticulin and MPL in MPN. Blood 131, 782-786 (2018).
65. Wolchok, et al., DNA vaccines: an active immunization strategy for prostate cancer. Semin Oncol 30, 659-666 (2003).

We claim:

1. An isolated heteroclitic CALR^{MUT} peptide comprising the amino acid sequence of any one of SEQ ID Nos 1-262.

2. A composition comprising a peptide according to claim 1.

3. A composition comprising a peptide according to claim 1 and a delivery vehicle.

4. The composition according to claim 3, wherein the delivery vehicle is selected from the group consisting of a nanoparticle, a lipid nanoparticle, a liposome, a lipid, a lipid encapsulation system, a polymer and a polymersome.

5. A composition comprising a peptide according to any of claims 2-4, further comprising an adjuvant.

6. An isolated nucleic acid molecule comprising a nucleic acid sequence encoding a peptide according to claim 1.

7. An isolated nucleic acid molecule comprising: (a) a nucleic acid sequence encoding a peptide according to claim 1, and (b) a nucleic acid sequence encoding a signal peptide, wherein the nucleic acid sequence encoding the peptide according to claim 1 is downstream of the nucleic acid sequence encoding the signal peptide.

8. The nucleic acid molecule according to claim 6 or 7, wherein the nucleic acid molecule is a DNA molecule.

9. The nucleic acid molecule according to claim 8, wherein the nucleic acid molecule comprises a promoter that is operably linked to the nucleic acid sequence encoding the peptide.

10. The nucleic acid molecule according to claim 6 or claim 7, wherein the nucleic acid molecule is an RNA molecule.

11. The nucleic acid molecule according to claim 10, wherein the nucleic acid molecule is an mRNA molecule.

12. A vector comprising a nucleic acid molecule according to any one of claims 6-11.

13. The vector according to claim 12, wherein the vector is a viral vector.

14. The viral vector according to claim 13, selected from the group consisting of an adenovirus, an adeno-associated virus, a retrovirus, a lentivirus, an alphavirus, and a vaccinia virus.

15. A cell comprising a nucleic acid molecule according to any of claims 6-11.

16. A composition comprising a nucleic acid molecule according to any of claims 6-11.

17. A composition comprising a nucleic acid molecule according to any of claims 6-11 and a delivery vehicle.

18. The composition according to claim 17, wherein the delivery vehicle is selected from the group consisting of a nanoparticle, a lipid nanoparticle, a liposome, a lipid, a lipid encapsulation system, a polymer and a polymersome.

19. The composition according to any of claims 16-18, further comprising an adjuvant.

20. An isolated heteroclitic CALR^{MUT} peptide comprising the amino acid sequence:
(SEQ ID NO. 275)
X1X2M3X4X5X6X7X8X9
wherein, independently of each other,
X1 is selected from: R, Y, F, M, and W,
X2 is selected from: M, Y, P, S, T, A, E, R, Q, F, and W,
X4 is selected from: R, D, and E,
X5 is selected from: T, W, Y, H, K, R, and F,
X6 is selected from: K, F, I, L, V, M, W, Y, T, C, N, and S,
X7 is selected from: M, and W,
X8 is selected from: R, A, P, S, Y, and F, and
X9 is selected from: M, K, V, F, R, Y, W, and H, and
wherein, the amino acid sequence comprises at least one point mutation as compared to CALR9p2 (SEQ ID NO. 263).

21. The peptide according to claim 20, wherein the amino acid sequence is selected from the group consisting of SEQ ID Nos 1-46.

22. The peptide according to claim 20, wherein the amino acid sequence is selected from the group consisting of SEQ ID Nos 1, 2, 4, 5, 6, 8 and 40.

23. A composition comprising a peptide according to any of claims 20-22.

24. A composition comprising a peptide according to any of claims 20-22 and a delivery vehicle.

25. The composition according to claim 24, wherein the delivery vehicle is selected from the group consisting of a nanoparticle, a lipid nanoparticle, a liposome, a lipid, a lipid encapsulation system, a polymer and a polymersome.

26. A composition according to any of claims 23-25, further comprising an adjuvant.

27. An isolated nucleic acid molecule comprising a nucleic acid sequence encoding a peptide according to any of claims 20-22.

28. An isolated nucleic acid molecule comprising: (a) a nucleic acid sequence encoding a peptide according to any of claims 20-22, and (b) a nucleic acid sequence encoding a signal peptide, wherein the nucleic acid sequence encoding the peptide according to any of claims 20-22 is downstream of the nucleic acid sequence encoding the signal peptide.

29. The nucleic acid molecule according to claim 27 or 28, wherein the nucleic acid molecule is a DNA molecule.

30. The nucleic acid molecule according to claim 29, wherein the nucleic acid molecule comprises a promoter that is operably linked to the nucleic acid sequence encoding the peptide.

31. The nucleic acid molecule according to claim 27 or claim 28, wherein the nucleic acid molecule is an RNA molecule.

32. The nucleic acid molecule according to claim 31, wherein the nucleic acid molecule is an mRNA molecule.

33. A vector comprising a nucleic acid molecule according to any one of claims 27-32.

34. The vector according to claim 33, wherein the vector is a viral vector.

35. The viral vector according to claim 34, selected from the group consisting of an adenovirus, an adeno-associated virus, a retrovirus, a lentivirus, an alphavirus, and a vaccinia virus.

36. A cell comprising a nucleic acid molecule according to any of claims 27-32.

37. A composition comprising a nucleic acid molecule according to any of claims 27-32.

38. A composition comprising a nucleic acid molecule according to any of claims 27-32 and a delivery vehicle.

39. The composition according to claim 38, wherein the delivery vehicle is selected from the group consisting of a nanoparticle, a lipid nanoparticle, a liposome, a lipid, a lipid encapsulation system, a polymer and a polymersome.

40. The composition according to any of claims 37-39, further comprising an adjuvant.

41. A method of treating a JAK2^{V617F} mutant-negative myeloproliferative neoplasm (MPN) in a subject in need thereof, the method comprising administering to the subject an effective amount of: (a) a heteroclitic CALR^{MUT} peptide according to claim 1, or (b) a nucleic acid molecule encoding a heteroclitic CALR^{MUT} peptide according to claim 1.

42. A method of treating a JAK2^{V617F} mutant-negative MPN in a subject in need thereof, the method comprising administering to the subject an effective amount of: (a) a heteroclitic CALR^{MUT} peptide according to claim 20, or (b) a nucleic acid molecule encoding a heteroclitic CALR^{MUT} peptide according to claim 20.

43. A method of treating a JAK2^{V617F} mutant-negative MPN in a subject in need thereof, the method comprising administering to the subject an effective amount of: (a) a heteroclitic CALR^{MUT} peptide according to claim 21, or (b) a nucleic acid molecule encoding a heteroclitic CALR^{MUT} peptide according to claim 21.

44. A method of treating a JAK2^{V617F} mutant-negative MPN in a subject in need thereof, the method comprising administering to the subject an effective amount of: (a) a heteroclitic CALR^{MUT} peptide according to claim 22, or (b) a nucleic acid molecule encoding a heteroclitic CALR^{MUT} peptide according to claim 22.

45. A method of treating a JAK2^{V617F} mutant-negative MPN in a subject in need thereof, the method comprising administering to the subject an effective amount of:
a. a heteroclitic CALR^{MUT} peptide according to any of claim 1, 20, 21 or 22,
b. a nucleic acid molecule according to any of claim 6-11 or 27-32,
c. a composition according to any of claim 2-5, 16-19, 23-26, or 37-40, or
d. a vector according to any of claim 12-14 or 33-35.

46. The method of any of claims 40-45, further comprising performing a diagnostic test to determine if the subject has a JAK2^{V617F} mutant-negative MPN.

47. The method of any of claims 40-46, further comprising administering to the subject an effective amount of an immune checkpoint inhibitor.

48. The method of claim 47, wherein the immune checkpoint inhibitor is a PD-1, PD-L1, PD-L2 or CTLA-4 inhibitor.

49. The method of claim 47, wherein the immune checkpoint inhibitor is anti-PD1 antibody.

50. The method of any of claims 40-49, wherein the treatment results in one or more of:
a. an immune response to the JAK2^{V617F} mutant-negative MPN,
b. A CD8+ T cell response to the JAK2^{V617F} mutant-negative MPN,
c. an anti-CALR^{MUT} immune response,
d. an anti-CALR^{MUT} T cell response,
e. enhanced sensitivity of the to the JAK2^{V617F} mutant-negative MPN to immune checkpoint blockade.
